(19) United States
(12) Patent Application Publication
(10) Pub. No.: US 2024/0082135 A1
(43) Pub. Date: Mar. 14, 2024

(54) PEPTIDE COMPOSITION AND RESPECTIVE USES

(71) Applicant: Universidade do Minho, Braga (PT)

(72) Inventors: Artur Manuel CAVACO PAULO, Braga (PT); Celia FREITAS DA CRUZ, Guimaraes (PT); Margarida Maria MACEDO FRANCESKO FERNANDES, Braga (PT)

(21) Appl. No.: 18/497,900

(22) Filed: Oct. 30, 2023

Related U.S. Application Data
(62) Division of application No. 18/334,287, filed on Jun. 13, 2023.
Application No. 18/334,287 is a continuation of application No. 18/194,372, filed on Mar. 31, 2023.
Application No. 18/194,372 is a continuation of application No. 16/439,889, filed on Jun. 13, 2019, now Pat. No. 11,642,298.
Application No. 16/439,889 is a continuation of application No. 15/030,313, filed on Apr. 18, 2016, now Pat. No. 10,709,655.

(30) Foreign Application Priority Data
Oct. 18, 2013 (PT) 107244

(51) Int. Cl. A61K 8/64; A61Q 5/00; A61Q 5/04; A61Q 5/06

(52) U.S. Cl. CPC A61K 8/64; A61K 8/645; A61Q 5/002; A61Q 5/04; A61Q 5/06; A61Q 5/065; A61K 2800/30

(57) ABSTRACT

The current application discloses a composition that comprises at least one peptide with a sequence length of 6-12 amino acids, where 2-5 of those amino acids are cysteines for the treatment and cosmetics of animal hair, in preference human hair. There are several hair styling methods that involve breakage and reestablishment of disulfide bonds, allowing relaxation and straightening of the hair. However, the most effective methods currently used to modulate hair contain harmful chemicals. Thus, there is a constant demand for formulations that efficiently model the hair fiber without damage. Thus, the present invention aims to provide a composition for treatment of the hair, including animal and human hair, without the use of chemicals harmful to the hair fiber and consumer health and uses of said compositions in shampoo, lotion, serum, cream, conditioner, foam, elixir, oil, aerosol or mask.

### CROSS-REFERENCE

This application is a divisional of 18/334,287, filed Jun. 13, 2023, which is a continuation of U.S. application Ser. No. 18/194,372, filed Mar. 31, 2023, which is a continuation of U.S. application Ser. No. 16/439,889, filed Jun. 13, 2019, now U.S. Pat. No. 11,642,298, issued May 9, 2023, which is a continuation of U.S. application Ser. No. 15/030,313, filed Apr. 18, 2016, now U.S. Pat. No. 10,709,655, issued Jul. 14, 2020, which is a U.S. National Stage Entry of International Application PCT/IB2014/065375, filed Oct. 16, 2014, which claims priority to Portuguese Application No. 107244, filed Oct. 18, 2013, all of which are hereby incorporated by reference in their entirety.

### SEQUENCE LISTING

The instant application contains a Sequence Listing which has been submitted electronically in XML format and is hereby incorporated by reference in its entirety. Said XML copy, created on Feb. 2, 2023, is named 63230-710-401SL.xml and is 1,068.431 bytes in size.

### TECHNICAL HELD

The current application corresponds to a composition that comprises at least one peptide, based on keratin and keratin associated proteins, containing 2 to 5 cysteines with the purpose of treatment and cosmetics of animal hair, in preference human hair.

### BACKGROUND

Human hair has a significant social role in most of the various world cultures, particularly for female population. Thus, there is a constant desire to improve and change hair characteristics, such as its natural texture. There are several differences in hair characteristics between different human ethnicities, as well as between individuals of the same ethnicity, such as length, thickness, color and texture.

Hair is composed of approximately 65% to 95% protein. The remaining constituents include water, lipids, pigments and trace elements. The majority of the proteins present in human hair correspond to keratin and keratin-associated proteins.

Human hair fiber's structure consists of cuticle, cortex and medulla. The cuticle constitutes about 15% by weight of the hair and consists of overlapping layers of cells, similar to a system of scales, with high content of cysteine. It provides a protective character to the hair fiber. The cortex is the middle region of the hair being responsible for the strength, elasticity and hair color. It is composed of several cell types and represents about 80% of the weight of the hair. The medulla corresponds to a central beam of cells and is absent in some hairs.

Keratins and mainly keratin-associated proteins have high sulfur content, present in the cysteine amino acid. The presence of sulfur is essential to the hair structure, as it allows the formation of disulfide bonds between amino acids of the polypeptide chains, due to oxidation of cysteine. The existence of these bonds is largely responsible for the structure and texture of the hair.

There are several hair styling methods that involve breakage and reestablishment of disulfide bonds, allowing relaxation and straightening of the hair. However, the most effective methods currently used to modulate hair contain harmful chemicals such as sodium hydroxide, potassium hydroxide, lithium hydroxide, guanidine hydroxide, ammonium thioglycolate or sodium sulfate. These methods can damage the scalp and the hair fiber, leading to its weakening and reducing its tensile strength. Formaldehyde, an extremely toxic chemical, is also used in hair straightening products. Other hair treatments that do not involve so much damage to the hair and the consumer are usually very expensive, time-consuming and/or have low efficacy. Thus, there is a constant demand for formulations that efficiently model the hair fiber without damage.

Peptides, proteins, amino acids and its derivatives have also been used in compositions for personal care products, namely hair conditioning and strengthening. For example, the document WO 00/23039 discloses a composition for hair treatment containing intermediate filament proteins, namely artificial keratin. The document EP 0488242 discloses a hair treating agent containing 3% to 10% by weight of cysteine and salts thereof, a polyhydric alcohol or a saccharide containing four to twenty carbon atoms, three or more hydroxyl groups in the molecule and no aldehyde or ketone group,

The current invention is distinguished by the use of peptides, while the other applications refer the use of, respectively, proteins and amino acids in isolation and together with other types of compounds. The peptides in this innovation peptide can penetrate into the Human hair in order to improve hair fiber resistance.

The document WO 00/51556 discloses a hair treatment composition that contains four or more discrete amino acids selected from histidine, lysine, methionine, tyrosine, tryptophan or cysteine. This document describes peptides without referring sequences and providing a composition essentially based on histidine, lysine, methionine, tyrosine, tryptophan or cysteine.

The document PT 103484 describes a formulation for cosmetic applications that uses hydrophobic binding domains and/or carbohydrates, in order to enhance its properties and to repair hair damage. The binding domains used are hydrolyzed milk protein, a model of human surfactant protein as well as biologically active and synthetic peptides. The current invention is distinguished by the innovative use of synthetic peptide sequences analogous to keratin proteins instead of surfactant proteins. Furthermore, it does not rely on hydrophobic binding domains and/or carbohydrates, but in other interactions, namely disulfide bonds.

Enzymes have also been used as activating agents for hair treatment, such as in the document WO 00/64405. The document WO 2012/13593 discloses a cosmetic kit for hair conformational change that acts specifically in the disulfide bonds of the hair keratin, through enzyme activating agents and proteolytic enzymes.

As described in the last document there are hair treatments that include actions at the level of the hair disulfide bonds. Below we highlight some examples.

The document WO 97/11672 reports a method for permanent hair processing using tris(2-carboxyethyl)phosphine (TCEP), and other water-soluble tertiary phosphines to break disulfide bonds, whose reaction occurs in acidicic environment. The document U.S. Pat. No. 5,635,170 discloses a composition for permanent shaping of hair based on a keratin reducing agent, which contains N-glycyl-L-cysteine and/or L-cysteinyl-glycine. The pH range of this composition is 6.5 to 9.0. The document WO 2008/081348 refers a method and composition for permanent modulation of hair, through the use of 1% to 30% of N-alkyl-2-mercapto acetamide as a keratin reducing agent. It also contains at least one cationic surfactant for permanently shaping hair and the resulting process. The document WO 2006/001536 describes an agent for permanent hair processing that contains a derivative of mercaptocarboxylic acid, which allows processing and reduction of hair keratin in the acidic and neutral range of the pH. The document US 2010/0272666 discloses a hair cosmetic composition for hair treatment, containing 5 to 50 amino acids, without containing cysteine or its derivatives. Thus, this invention is distinguished by the existence of specific amino acid sequences, which contain cysteine, allowing the formation of disulfide bonds that stabilize and protect the hair fiber.

In a previous article by Fernandes et al. (Fernandes, Lima, Loureiro, Gomes, & Cavaco-Paulo, 2012), it is performed the toxicology evaluation of a peptide sequence for hair care use, containing 13 amino acids with two cysteines in its composition. However, in this article it is not mentioned or suggested that the percentage of cysteine in a peptide sequence may have some effect on the resistance of the hair. Also, in the present innovation, the number of amino acids of each peptide sequence is 6 to 12.

### SUMMARY

Thus, the present invention aims to provide a composition for treatment of the hair, including animal and human hair, without the use of chemicals harmful to the hair fiber and consumer health and that does not present the drawbacks found in the state of the art.

The compositions described in the current invention, after prolonged use, provide hair with soft, shiny, undamaged texture and with the desired features. The peptide compositions with a specific number of amino acids and cysteines act synergically providing resistance to strength, toughness and elasticity to the hair. Therefore, the compositions of the current invention are particularly relevant for hair treatment, hair dying, hair perms, etc.

The present application describes a peptide composition for hair treatment, in particular human or animal hair, which comprises at least one peptide with 6-12 amino acids length (namely 6, 7, 8, 9, 10, 11, 12 amino acids), where 2-5 of those amino acids correspond to cysteine, preferably 2, 3, 4 or 5 of those amino acids are cysteines and dermatologically suitable excipients, which penetrates the hair, increasing it resistance and reducing it breakage.

In the embodiment, for improved results, the peptide (or peptides) of the peptide composition for hair care can comprise 10-11 amino acids.

In the embodiment of the peptide composition for hair care treatment, the referred peptides can also contain a percentage of hydrophobic amino acids, not higher than 60%, and preferably less than 41% for better results. Preferably, the composition can also comprise at least one hydrophobic amino acid selected from the following list: phenylalanine, alanine, leucine, methionine, isoleucine, tryptophan, proline, valine or their mixtures.

In yet another embodiment, the amount of cysteine of the peptide composition for hair treatment may vary from 10% to 50% of the total of amino acids of the peptide sequence, preferably 20-30%, and even more preferably 25%.

In an embodiment of the composition, with better results of the peptide (or peptides) of the peptide composition for hair treatment, the sequence of peptide(s) can comprise at least one sequence of the following list with a with a degree of homology greater than or equal to 90%: SEQ. ID NO: 1-SEQ. ID NO: 1239, preferably with a degree of homology greater than or equal to 91%, 92%, 93%, 94%, 95%, 96%, 97%, 98%, 99%, 100%.

In an embodiment, improved results for the peptide (or peptides) of the peptide composition for hair treatment can comprise at least one of the sequences of the following list with a degree of homology equal or greater than 90%: SEQ.ID NO:5, SEQ.ID NO:75; SEQ.ID NO:94; SEQ.ID NO:409; SEQ.ID NO:411; SEQ.ID NO:412; SEQ ID. NO:432; SEQ.ID NO:618; SEQ.ID NO:717; SEQ.ID NO:951; SEQ.ID NO:1088; SEQ.ID NO:1131; SEQ.ID NO:1149, preferably with a degree of homology equal or greater than 91%, 92%, 93%, 94%, 95%, 96%, 97%, 98%, 99%, 100%.

In other embodiment, the concentration of the peptide of the peptide composition for hair treatment can vary between 0.001%-20% (w/w), preferably 0.01-5% w/w).

In yet other embodiment, the peptide composition for hair treatment can comprise at least one excipient, selected from the following list: surfactants, emulsifiers, preservatives, thickeners, organic polymers, humectants, silicones, oils, fragrances, vitamins, buffers.

In another embodiment, the peptide composition for hair treatment can compose at least one anionic surfactant selected from the following list: alkylbenzene sulfonates, ammonium lauryl sulfate, ammonium lauryl sulfate, ammonium xylenesulfonate, sodium C14-16 olefin sulfonate, sodium cocoyl sarcosinate, sodium laureth sulfate, sodium lauryl sulfate, sodium lauryl sulfoacetate, sodium myreth sulfate, sodium xylenesulfonate, TEA-dodecylbenzenesulfonate, ethyl PEG-15 cocamine sulfate, dioctyl sodium sulfosuccinate, or any mixture thereof.

In an embodiment, the peptide composition for hair treatment can comprise at least one amphoteric surfactant selected from the following list: cocamidopropyl betaine, coco betaine, cocoamphoacetate, cocoamphodipropionate, disodium cocoamphodiacetate, disodium cocoamphodipropionate, lauroamphoacetate, sodium cocoyl isethionate, or any mixture thereof.

In other embodiment, the peptide composition for hair treatment can comprise at least one cationic surfactant selected from the following list: quaternary ammonium compounds, behentrimonium chloride, behentrimonium methosulfate, benzalkonium chloride, betrimonium chloride, binnamidopropyltrimonium chloride, cocotrimonium chloride, dicetyldimonium chloride, dicocodimonium chloride, dihydrogenated tallow dimethylammonium chloride, hydrogenated Palm trimethylammonium chloride, laurtrimonium chloride, quatemium-15, quaternium-18 bentonite, quatemium-22 hectonite, stearalkonium chloride, tallowtrimonium chloride, tricetyldimonium chloride, or any mixture thereof.

In yet other embodiment, the peptide composition for hair treatment can comprise at least one non-ionic surfactant selected from the following list: decyl glucoside, laureth-10 (lauryl ether 10), laureth-23, Laureth-4, PEG-10 sorbitan laurate, polysorbate-(20, 21, 40, 60, 61, 65, 80, 81), PPG-1 trideceth-6, sorbitol, steareth-(2, 10, 15, 20), C11-21 pareth-(3-30), C12-20 acid PEG-8 ester, or their mixtures.

In yet other embodiment, the peptide composition for hair treatment can comprise at least one emulsifier selected from the following list: caprylic/capric/diglyceryl succinate, C10-15 pareth-(2,4,6,8) phosphate, C14-16 glycol palmitate, C18-20 glycol isostearate, ceteareth-(4-60), cocamidopropyl lauryl ether, deceth-(3-10), DTPA-hydrogenated cocoate, dipentaerythrityl. hydroxystearate, dipentaerythrityl hydroxyisostearate, dipentaerythrityl hexacapratelcaprylate, dodoxynol-(5,6,7,9,12), nonoxynol-(1-35), octoxynol-(1-70), Octyldodeceth-(2,5,16,20,25), Palm kernel glycerides, or any mixture thereof.

In other embodiment, the peptide composition for hair treatment can comprise at least one preservative selected from the following list: butyl paraben, diazolidinyl urea, DMDM hydantoin, ethyl paraben, imidazolidinyl urea, iodopropynyl butylcarbamate, isobutyl paraben, methyl paraben, methylchloroisothiazolinone, methylisothiazolinone, phenoxyethanol, propyl paraben, sodium benzoate, or any mixture thereof.

In other embodiment, the peptide composition for hair treatment can comprise at least one thickener selected from the following list: aluminum stearates/isostearates/myristates/laurates/palmitates, glycol distearate, hydrogenated castor oil, hydrogenated castor oil hydroxystearate, hydrogenated castor oil isostearate, hydrogenated castor oil stearate, hydrogenated castor PEG-8 esters, PEG-150 distearate, or any mixture thereof.

In other embodiment, the peptide composition for hair treatment can comprise at least one natural polymer derived selected from the following list: carboxymethyl hydroxyethyl celulose, carboxymethyl hydroxypropyl guar, cellulose, ethyl celulose, hydroxy-butyl methylcellulose, hydroxyethylcellulose, hydroxymethylcellulose, lauryl polyglucose, or any mixture thereof.

In other embodiment, the peptide composition for hair treatment can comprise at least one humectant selected from the following list: 1,2,6 hexanetriol, dipropylene glycol, glycerin, hexylene glycol, panthenol, phytantriol, propylene glycol, sodium PCA, sorbitol, triethylene glycol, polyglyceryl sorbitol, glucose, fructose, polydextrose, potassium PCA, hydrogenated honey, hyaluronic acid, inositol, hexanediol beeswax, hexanetriol beeswax, hydrolyzed elastin, hydrolyzed collagen, hydrolyzed silk, hydrolyzed keratin, erythritol, capryl glycol, isoceteth-(3-10, 20, 30), isolaureth-(3-10, 20, 30), laneth-(5-50), laureth-(1-30), steareth-(4-20), trideceth-(5-50), or any mixture thereof.

In other embodiment, the peptide composition for hair treatment can comprise at least one cationic polymer selected from the following list: polyquatemium-10, polyquatemium-7, polvquaternium-11m guar hydroxypropyltrimonium chloride, or any mixture thereof.

In other embodiment, the peptide composition for hair treatment can comprise at least one silicone selected from the following list: amodimethicone, amodimethicone, trideceth-12, cetrimonium, chlotide mixture, behenoxy , dimethicone sparingly, cetearyl methicone, cetyl dimethicone, cyclomethicone, cyclopentasiloxane, dimethicone, dimethicone copolyol, dimethicone copolyol, dimethiconol, hydrolyzed wheat protein hydroxypropyl polysiloxane, stearoxy dimethicone sparingly, stearyl dimethicone, trimethylsitylamodimethicone, lauryl methicone copolyol, or any mixture thereof.

In yet other embodiment, the peptide composition for hair treatment can comprise at least one organic oil selected from the following list: mineral oil, paraffin, petrolatum, or any mixture thereof.

In yet other embodiment, the peptide composition for hair treatment can comprise at least one protein selected from the following list: cocodimonium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, cocoyl hydrolyzed collagen, cocoyl hydrolyzed keratin, hydrolyzed keratin, hydrolyzed oat flour, hydrolyzed silk, hydrolyzed silk protein, hydrolyzed soy protein, hydrolyzed wheat protein, hydrolyzed wheat protein, keratin, potassium cocoyl hydrolyzed collagen, TEA-cocoyl hydrolyzed collagen, TEA-cocoyl hydrolyzed soy protein, or any mixture thereof.

In other embodiment, the peptide composition for hair treatment can comprise at least one vitamin selected from the following list: retinol, retinyl palmitate tocopherol acetate, or any mixture thereof.

In other embodiment, the peptide composition for hair treatment can comprise at least one ester emollient selected from the following list: butyl myristate, butyl stearate, C12-15 alkyl benzoate, caprylicicapric triglyceride, cetyl octanoate, cetyl stearate, cetearyl stearate, decyl oleate, dimethyl lauramine isostearate, glyceryl stearate, glyceryl adipate, glyceryl arachidate, glyceryl arachidonate, glyceryl behenate, glyceryl caprate, glyceryl caprylate, glyceryl caprylate/caprate, glyceryl citrate/lactate/linoleate/oleate, glyceryl cocoate, glyceryl diarachidate, glyceryl dibehenate, glyceryl dierucate, glyceryl dihydroxystearate, glyceryl diisopalmitate, glyceryl diisostearate, glyceryl dilaurate, glyceryl dilinoleate, glyceryl dimyristate, glyceryl dioleate, glyceryl dipalmitate, glyceryl dipalmitoleate, glyceryl diricinoleate, glyceryl distearate, glyceryl erucate, glycol stearate, isocetyl stearate, isopropyl myristate, isopropyl palmitate, isopropyl stearate, isostearyl stearate, octyl palmitate, octyl stearate, propylene glycol dicaprylate/dicaprate, sorbitan benzoate, sorbitan caprylate, sorbitan isostearate, Sorbitan laurate, sorbitan tristearate, stearyl stearate, tocopheryl linoleate, or any mixture thereof.

In other embodiment, the peptide composition for hair treatment can comprise at least one alkanolamide selected from the following list: acetamide MEA, cocamide DEA, cocarnide MEA, lactamide MEA, lauramide DEA, lauramide DEA, propylene glycol, lauramide MEA, lecithinamide DEA, linolearnide DEA, linolearnide MEA, linoleamide MIPA, myristamide DEA, myristamide MEA, myristamide MIPA, oleamide DEA, oleamide DEA, oleamide MEA, oleamide MIPA, soyamide DEA, stearamide MEA, or any mixture thereof.

In yet other embodiment, the peptide composition for hair treatment can comprise at least one amine selected from the following list: behentamidopropyl dimethylamine, cocamidopropyl dimethylamine, isostearamidopropyl dimethylamine, lauramidopropyl dimethylamine, myristamidopropyl dimethylamine, oleamidopropyl dimethylamine, palmitamidopropyl dimethylamine, stearamidopropyl dimethylamine, tallamidopropyl dimethylamine, or any mixture thereof.

In yet other embodiment, the peptide composition for hair treatment can comprise at least one pH adjuster selected from the following list: ascorbic acid, citric acid, sodium hydroxide, triethanolamine, or any mixture thereof.

In yet other embodiment, the peptide composition for hair treatment can comprise at least one salt selected from the following list: calcium chloride, magnesium chloride, magnesium sulfate, potassium chloride, potassium glycol sulfate, sodium chloride, or any mixture thereof.

In yet other embodiment, the peptide composition for hair treatment can comprise at least one aliphatic alcohol selected from the following list: behenyl alcohol, cetearyl alcohol, cetyl alcohol, isocetyl alcohol, isostearyl alcohol, lauryl alcohol, myristyl alcohol, stearyl alcohol, C30-50 alcohols, lanolin alcohol, or any mixture thereof.

In another embodiment, the peptide composition for hair treatment can comprise at least one UV filter/sunscreen selected from the following list: benzophenone-(2, 3, 4, 5, 6, 7, 8, 9, or 10), benzophenone-4, benzyl salicylate, benzylidene camphor sulfonic acid, bornelone, ethyl cinnamate, ethylhexyl methoxycinnamate (octyl methoxycinnamate), octoxynol-40, octoxynol-20, octyl methoxycinnamate, octyl salicylate, oxybenzone, phenyl ketone, PEG-25 PABA, polyacrylamidomethyl benzylidene camphor, or any mixture thereof.

In other embodiment, the peptide composition for hair treatment can comprise at least one natural oil selected from the following list: coconut oil, jojoba oil, olive oil, palm Oil, safflower oil, sesame seed oil, shea butter, sweet almond oil, wheat germ oil, or any mixture thereof.

In yet other embodiment, the peptide composition for hair treatment can comprise at least one amine oxide selected from the following list: cocamine oxide, lauramine oxide, or any mixture thereof.

In other embodiment, the peptide composition for hair treatment can comprise at least one chelate selected from the following list: diiospropyl oxalate, disodium EDTA, disodium EDTA-copper, HEDTA, oxalic acid, potassium citrate, sodium citrate, dodium oxalate, TEA-EDTA, tetrasodium EDTA, trisodium EDTA, trisodium HEDTA, or any mixture thereof.

In other embodiment, the peptide composition for hair treatment can comprise at least one fatty acid selected from the following list: arichidonic acid, capric acid, coconut fatty acid, lauric acid, linoleic acid, linolenic acid, myristic acid, palmitic acid, pantothenic acid, stearin; acid, caproic acid, capryleth-(4, 6, 9) carboxylic acid, isostearic acid, or any mixture thereof.

In other embodiment, the peptide composition for hair treatment can comprise at least one agent antimicrobial/antibacterial selected from the following list: glyoxal, triclosan, or any mixture thereof.

In other embodiment, the peptide composition for hair treatment can comprise at least one PEG-modified material selected from the following list: PEG-150 pentaerythirtyl tetrastearate, PEG-(-2, -3, -4, -6, -8, -12, -20, -32, -50, -150, -175) distearate, PEG-10 castor oil, PEG-10 cocamine, PEG-10 cocoate, PEG-10 coconut oil esters, PEG-10 glyceryl oleate, PEG-10 glyceryl pibsa tallate, PEG-10 glyceryl stearate, PEG-10 hydrogenated lanolin, PEG-10 hydrogenated tallow amine, PEG-10 isolauryl thioether, PEG-10 isostearate, PEG-10 lanolate, PEG-10 lanolin, PEG-10 laurate, PEG-10 oleate, PEG-10 olive glycerides, PEG-10 polyglyceryl-2 laurate, PEG-10 propylene glycol, PEG-10 sorbitan laurate, PEG-10 soya sterol, PEG-10 soyamine, PEG-10 stearamine, PEG-10 stearate, PEG-10 stearyl benzonium chloride, PEG-10 tallate, PEG-10 tallow aminopropylarnine, PEG-100, PEG-100 castor oil, PEG-100 hydrogenated castor oil, PEG-100 lanolin, PEG-100 stearate, PEG-40 hydrogenated castor Oil, PEG-60, PEG-55 propylene glycol distearate, or any mixture thereof.

In other embodiment, the peptide composition for hair treatment can comprise at least one polymer selected from the following list: carbomer, dodecanedioic acid/cetearyl alcohol/glycol copolymer, hydrogenated C6-14 olefin polymers, hydrogenated ethylene/propylene/styrene copolymer: polyacrylic acid, polymethyl methacrylate: polymer, polyvinyl acetate, polyvinyl alcohol, PPG, PPG-25-laureth-25, PPG-5 pentaerithrityl ether, PPG-75-PEG-300-hexylene glycol, polyvinylpyrrolidone, PVP/VA (polyvinylpyrrolidone/vinyl acetate copolymer), sodium carbomer, TEA-carbomer, poloxamer (100-407), poloxamine, polyacrylamidomethylpropane sulfonic acid, polyethylene terephthalate, or any mixture thereof.

In other embodiment, the peptide composition for hair treatment can comprise at least one antistatic agent selected from the following list: apricotamidopropyl ethyldimonium ethosulfate, apricotamidopropyl ethyldimonium lactate, cocamidopropyl ethyldimonium ethosulfate, cocamidopropyl ethyldimonium lactate, lauramidopropyl ethyldimonium ethosulfate, lauramidopropyl ethyldimonium lactate, linoleamidopropyl ethyldimonium ethosulfate, linoleamidopropyl ethyldimonium lactate, myristamidopropyl ethyldimonium ethosulfate, myristamidopropyl ethyldimonium lactate, oleamidopropyl ethyldimonium ethosulfate, olearnidopropyl ethyldimonium lactate, stearamidopropyl ethyldimonium ethosulfate, stearamidopropyl ethyldimonium lactate, or any mixture thereof.

In other embodiment, the peptide composition for hair treatment can comprise at least one alcohol selected from the following list: SD alcohol 40, witch hazel, isopropanol, or any mixture thereof.

In yet other embodiment, the peptide composition for hair treatment can comprise fragrances, oils or any mixture thereof.

In other embodiment, the peptide composition for hair treatment can be used in medicine, veterinary and/or for cosmetics, preferably for the treatment of hair, mainly for animal or human, particularly for treating diseases of the scalp, particularly scalp irritation, alopecia areata, lichen planus, folliculitis keloid of the neck, trichorrhexis nodosa, tricodistrophy, pili torti, tricorrexis

invaginata, moniletrix, uncombable hair syndrome.[0058] In other embodiment, the composition may comprise a dye agent linked to the N or C-terminal of the referred peptides.

In yet other embodiment is the use of the described composition for hair coloring.

Other aspect of the embodiment is the use of the described composition as a hair strengthener or as fixer of perms and/or curly hairs.

It is also described in this application shampoo, lotion, serum, cream, conditioner, foam, elixir, oil, aerosol or mask comprising the composition presented in this application.

The present application discloses a composition for hair treatment that comprise, in whole or in part, one or more peptide sequences of 6 to 12 amino acid residues based on keratin and keratin-associated proteins having 2 to 5 cysteine residues, preferably having 3 to 5 residues of cysteine, for treatment and cosmetics of the hair, preferably human hair, chemically pre-treated or not. Thus, the presence of cysteine in the peptide sequence (higher than 10%, preferably more than 15%) in combination with a percentage of hydrophobic amino acids ensures that the peptides can have a lasting fixation in the hair, improving the human hair properties such as elasticity and strength.

Surprisingly, the described peptide compositions in which the peptide(s) comprising 2 to 5 cysteines allow penetration of the peptide(s) and enhance the properties of hair, preferably 3-5 cysteines. Thus, described peptide(s) containing 2-5 cysteine in order to allow hair penetration and enrichment of the hair properties, such as elasticity, resistance, reduce eventual hair damage, as well as improve and change hair characteristics.

The peptide compositions described in the present application surprisingly enrich and improve the properties and characteristics of the hair, such as elasticity, strength and appearance, repairing damaged keratinous fiber. Therefore, formulation's high cysteine content is used to improve and/or change its characteristics, such as hair curl or uncurl. The sequence of peptides can have also preferably a percentage of hydrophobic amino acids not exceeding 60%, improving even further the results. Examples of hydrophobic amino acids are phenylalanine, alanine, leucine, methionine, isoleucine, tryptophan, proline, valine, and others.

In the context of the present description, the peptide composition can also be applied to the hair and in particular to the human hair as, but not limited to, aqueous solution or conventional shampoo or conditioner. It can also be used as a lotion, foam, aerosol, gel, mask, and application formulation with or without subsequent rinsing.

The concentration of peptide to be used depends on several features such as the condition of the hair, the origin and the formulation of the hair care product.

### DETAILED DESCRIPTION

It should be understood that the detailed description and specific examples are indicative of preferred embodiments of the invention and are provided to be illustrative only. This patent is not limited to those mentioned applications.

The present application describes a composition for hair treatment that comprises different peptides, which are based in the structure of keratin and keratin associated proteins.

The compositions described in the present application allow surprisingly the dermo-cosmetic treatment of animal hair, including human hair, chemically pre-treated or not. The composition described in the present invention, through the use of specific peptides, allows the preparation of keratinous fiber damages, due to the high binding capacity of the keratin peptides, including through disulfide bridges.

The described compositions improve the properties and characteristics of the hair, such as elasticity, resistance and appearance, repairing putative damages of the hair.

The peptides here defined are peptide sequences which bind with a certain affinity to the hair. The peptides used in this invention are composed by 6 to 12 amino acids and are constituted by a minimum of 2 and a maximum of 5 cysteines, preferably 3-5 cysteines.

The peptide composition for hair treatment described allows a resistance increase due to the presence of the cysteine-rich peptide, which leads to the resistance of the hair even after several rinsing.

Every peptide can be used together or separately, as well as all or part of the peptide sequence in the hair composition. Each peptide sequence contains amino acids with sulfur, specifically cysteine, which interacts with the hair and allows the formation of intermolecular cross-linking, stabilizing the keratinous fiber.

The peptide composition described uses a high content on cysteine in order to enrich the hair properties, such as improve elasticity and resistance, reduce putative damage of the hair, improve and/or change hair characteristics. Regarding the interaction with the keratinous fibers, the cysteine is 10% to 50% of the total amount of amino acids of the peptide sequence. Additionally, the number of amino acids of the peptide sequence is preferable from 6 to 12.

The peptides can be used separately or in combination of two or more peptides. The concentration of the peptide to be used depends on several characteristics, such as hair condition, origin and the formulation of the product for hair treatment. The content of the hair composition of the present invention is as example 1-0.001% (w/w) in mass.

The peptides of the present invention can be prepared by conventional methods of peptide synthesis, well known in the state of the art.

Additionally, many companies provide customized services for peptide synthesis.

An embodiment of the current invention describes peptides that link to the hair, and which sequence of amino acids includes cysteines where the sequence is selected from the group between the sequences ID NO: 1 to sequence ID NO: 1239.

The sequence of the 1239 peptides referred is listed in the table of the FIG. 1,

As example of hair, it was used virgin human hair tresses, acquired from the International Hair Importers and Products, Inc. (New York), The term virgin hair is applied to all the hair that was never subject or was at least 10 years without making any chemical treatment. Several different hair samples such as African, Asian and Caucasian hair are commercially available in several companies, such as the company mentioned above. Optionally, the hair samples can be treated, for example, using hydrogen peroxide to bleach the hair, needed for techniques such as hair dying.

In the context of this invention, the peptides can be applied to the hair, such as the human hair in the form of, but not limited to, aqueous or conventional preparation of shampoo or conditioner. It can also be in the form of lotion, foam, spray, gel, mask, formulation applied with or without subsequent rinsing.

This invention can be prepared by peptide coupling with an agent of these preparations directly or via a spacer.

This coupling interaction can be performed by covalent or non-covalent bonds, such as hydrogen bond, electrostatic interactions, hydrophobic interactions or van der Waals interactions. The spacer can be used to separate the peptide from the preparation agent, ensuring that the agent does not interfere with the peptide linkage to the hair.

The present invention can be understood more clearly and accurately by reading the following examples, which are indicative of preferred embodiments of the invention. They are provided for illustration in greater detail of the present invention, without introducing any limitation and without being limited to those applications.

### EXAMPLES

The examples that are within the scope of the claims represent different embodiments of the invention; all other examples are comparative examples.

### Example 1

The present application treats human hair through several commercial formulations with and without the use of the peptides from the sequence ID NO: 5. As The hair was supplied from International Hair Importers and Products, Inc. (New York).

The tests were performed with in human hair after 8 treatments of bleaching, at 50° C., in 0.1 M Na2CO3/NaHCO3 buffer, at 10% H2O2, for 1 hour.

Several formulations were tested:

hair serum with 15% PCG;

hair mask.

The mask used in this application was a basic commercial formulation with water, denaturing alcohol, propylene glycol, ether dicaprylic, cetylstearyl alcohol, behentrimonium chloride, cetyl ester, polysorbate 20, hydrolyzed wheat protein, hydrolyzed wheat starch, benzyl alcohol and fragrance.

The hair serum used in this application was a basic commercial formulation with water, denaturing alcohol, propylene glycol, polysorbate 20, hydrolyzed wheat protein, hydrolyzed wheat starch, crosslinked polymer alkyl acrylate/C10-30, triethanolamine, benzyl alcohol, fragrance.

Each of the formulations was tested with and without the peptide sequence ID NO:5, which contains in the sequence 15% of cysteine. The formulations containing the peptide SEQ ID NO:5 had a concentration of peptide of 0.1 mg/mL, in a ratio 1:1 (v/v).

To demonstrate the effect was also tested:

a peptide whose sequence does not contain cysteine, with approximately 41% hydrophobic amino acids;

a peptide which contains in it sequence 8% cysteine, with approximately 58% hydrophobic amino acids.

The hair mask was applied to the hair after 8 bleaching treatments, being left to act for 15 minutes, mimicking the procedure indicated in commercial masks. Posteriorly, the hair was washed. The serum was applied to the hair after 8 bleaching treatments, being left to act for 1 hour at 37° C. Posteriorly, the hair was not washed, as in typical commercial procedures the serum should be applied in dry hair. The hair was also tested after 5 applications.

The peptide from the sequence ID NO: 5 was able to penetrate in the hair fiber for all the formulations.

After the treatment, mechanical tests were performed, using a cell with 2.5 N maximum load and a deformation rate of 1.5 mm/min. Each hair was individually mounted in the tensile jig by means of a paper template with a fixed gauge length of 20 mm.

TABLE 1
Young modulus of virgin hair without treatments
and after 8 times bleaching treatments.

| Hair type | Young modulus (MPa) |
|---|---|
| Virgin hair | 6579 |
| Hair after 8 time bleaching | 5294 |
| Serum(with a 15% cysteine and 50% | 7149 |
| hydrophobic amino acids peptide) | |
| Serum for comparison(with a 41% | 6180 |
| hydrophobic amino acid without cysteine | |
| peptide) | |
| Serum for comparison | 6456 |
| (with a 8% cysteine and 58% | |
| hydrophobic amino acid peptide) | |
| Serum for comparison (without peptide) | 6034 |

TABLE 2
Young modulus for different types of hair treatment.

| | Young modulus | Young modulus |
|---|---|---|
| | after 1 | after 5 |
| | application | applications |
| Type of treatment | (MPa) | (MPa) |
| Serum (with a 15% cysteine | 7149 | 7318 |
| and 50% hydrophobic amino | | |
| acid peptide) | | |
| Serum for comparison | 6034 | 6112 |
| (without peptide) | | |
| Mask (with a 15% cysteine | 6175 | 7075 |
| and 50% hydrophobic amino | | |
| acid peptide) | | |
| Mask for comparison(without | 5514 | 5685 |
| peptide) | | |

The peptide in these treatments is the peptide from sequence ID NO: 5, The formulations which contain the sequence ID NO:5 induce an increase in mechanical resistance of the damaged hair. After 5 applications, the hair treated with the sequence ID NO: 5 maintain the high resistance, having a higher increase in the resistance than without the peptide.

### Example 2

This example discloses the treatment of human hair with peptides containing cysteine, and in this case the peptide containing the sequence ID NO: 409, based in the assumption that small peptides are able to penetrate in the hair fiber cuticle.

The hair was supplied from International Hair Importers and Products, Inc. (New York). Hair fibers were pre-treated by bleaching. The formulation was tested in different hair types:
virgin hair washed, with the cuticle intact and absence of chemical damages;
hair after 8 bleaching treatments, at 50° C. in 0.1 M Na2CO3/NaHCO3 buffer, at pH=9, 10% H2O2, for 1 hour.

The incorporation of the peptides was performed by direct application in the hair surface. The mechanical resistance tests were performed after the treatment of the hair with the peptide.

The measurements of mechanical resistance were performed using a cell with 2.5 N maximum load and a deformation rate of 1.5 mm/min. Each hair was individually mounted in the tensile jig by means of a paper template with a fixed gauge length of 20 mm.

As for the results obtained for the mechanical test showed that compared to the control, i.e., virgin hair without bleaching or peptide treatment (Young modulus: 4142±590 MPa), bleaching reduced the Young modulus (2478±567 MPa), while the treatment with the peptide sequence ID NO: 409 after bleaching increased the Young modulus to higher valued than the virgin hair with no treatment (5649±1022 MPa).

### Example 3

This example discloses the treatment of human hair with a composition comprising peptides. In this example, the peptide with the sequence ID NO: 412 was tested. The hair was supplied from International Hair Importers and Products, Inc. (New York).

The formulation was tested in different hair types:
virgin hair washed, with the cuticle intact and absence of chemical damages;
hair after reduction treatment, at 37° C. in phosphate buffer at pH=8, with 3M GndHCl and 0.05M DTT for 2 hours.

For the treatment with the peptide SEQ ID NO: 412, concentrations of 0.01% (w/w) were used.

The average of the Young's modulus for relaxed hair is 3002 MPa, while for relaxed hair fiber after peptide treatment at 0.01% is 4190 MPa. The Young modulus value for virgin hair without treatment is 5214 MPa.

In the maximum load test, for the relaxed hair fiber, the maximum resistance was 96 MPa, while for the hair fiber relaxed after peptide treatment 126 MPa and for the virgin hair with no treatment 203 MPa.

Regarding hair stretching, the relaxed hair has an average of 51%, while after treatment with the peptide sequence ID NO: 412, has a stretching of 72%. For virgin hair, the average of hair stretching is 58%.

Therefore, it is evident that the peptides are capable to prevent the hair surface degradation and consequently, the hair treated with these peptides has a longer life span.

### Example 4

In order to assess the interactions between the keratin and some peptides, a keratin solution was prepared. This procedure was performed by immersing African hair, acquired from the International Hair Importers and Products, Inc. (New York), in a solution containing 8 M urea, 0.2 M sodium dodecyl sulfate and 0.5 M sodium bisulfite. The mixture was heated to 50 ° C. for 24 h in a shaker bath. The solution was dialyzed for several days against double-distilled water. The keratin solution was then concentrated using AMICON with a 3 kDa cut-off The keratin was then conjugated with Alexa Fluor 647 carboxylic acid, succinimidyl ester in DMSO anhydrous 5%.

The reaction was incubated for 1 h 30 min at room temperature and in the dark. The Alexa Fluor 647 that did not link to the keratin solution was separated by centrifugation in AMICON with a 3 kDa cut-off for 1 h at 25° C. and 5000×g.

The keratin was then diluted to 1 lag./mL in blocking buffer (3% BSA in tris-buffered saline (TBS) with 0.05% Tween 20). The peptides tested were SEQ.ID NO: 179, SEQ. ID NO:75, SEQ.ID NO:432, SEQ.ID NO:951, SEQ.ID NO:1108, SEQ.ID NO: 1131 and a peptide containing 13 amino acids, including 2 cysteines (X3CX5CX3), where X represents one of known amino acid residues, with the exception of cysteine residue that is represented by the letter C. This peptide is similar to the one tested in Fernandes et at (Fernandes, Lima, Loureiro, Goines, & Cavaco-Paulo, 2012).

Several peptides in a concentration of 15 fmol/mm2, were attached to a glass through a hydrophilic linked moiety, and were then incubated with the keratin, marked with Alexa Fluor 647, for 2 hours at 37° C.

After incubation, the glasses were rinsed in successive washing solutions: TBS+0.1% Tween 20 and blocking buffer with 3%BSA in TBS+0.1% Tween 20, for 3 minutes in each solution.

The imaging of the glasses was performed in Agilent G2565CA Microarray Scanner System. Three replicas of each peptide incubation were performed and analyzed.

TABLE 3
Normalized intensity levels of peptide sequences.

| | | | Hydrophobic | Intensity level |
|---|---|---|---|---|
| | | | amino | (average ± |
| | Number of | Cysteine | acids | standard |
| Sequence | amino acids | content | content | deviation) |
| SEQ. ID | 10 | 20% | 50% | 0.990 ± 0.014 |
| NO: 179 | | | | |
| SEQ. ID | 10 | 30% | 60% | 1.000 ± 0.000 |
| NO: 75 | | | | |
| SEQ. ID | 10 | 30% | 40% | 1.000 ± 0.000 |
| NO: 432 | | | | |
| SEQ. ID | 10 | 40% | 30% | 1.000 ± 0.000 |
| NO: 951 | | | | |
| SEQ. ID | 11 | 46% | 18% | 1.000 ± 0.000 |
| NO: 1108 | | | | |
| SEQ. ID | 11 | 46% | 9% | 1.000 ± 0.000 |
| NO: 1131 | | | | |
| X₃CX₅CX₃ | 13 | 15% | 38% | 0.184 ± 0.084 |

The peptides SEQ.ID NO:75, SEQ.ID NO:432, SEQ.ID NO:951, SEQ.ID NO:1108, SEQ.ID NO:1131, with percentage of cysteine ranging from 30% to 46%, such as and percentage of hydrophobic amino acids ranging from 9% to 60% were able to obtain an intensity of 1, indicating a very high affinity to keratin. The peptide SEQ.ID NO:179, with 20% and 50% of cysteine and hydrophobic content, respectively showed a slightly inferior but still very high intensity (0.990+0.014). These peptides were compared with a peptide similar to the one described in Fernandes et al. (Fernandes, Lima, Loureiro, Gomes, & Cavaco-Paulo, 2012) containing 2 cysteines in a 13 amino acids sequence. The reduced percentage of cysteine (15%) and higher number of amino acids in the sequence (13 amino acids) lead to a decrease in the intensity to 0.184±0.084, showing an inferior affinity to keratin. This suggests that the higher number of amino acids difficult the reaction of the peptide with the hair keratins, This inferior affinity to keratin leads to less fixation of the peptides in the hair in posterior treatments and consequently providing less improvements in the recovery of the hair characteristics.

The sequences of peptides are described by one letter code of amino acids. The code is as follows:

List of peptide sequences

| Amino acid - One Letter Code | |
|---|---|
| Histidine - H | |
| Arginine - R | |
| Lysine - K | |
| Isoleucine - I | |
| Phenylalanine - F | |
| Leucine - L | |
| Tryptophan - W | |
| Alanine - A | |
| Methionine - M | |
| Proline - P | |
| Valine - V | |
| Cysteine - C | |
| Asparagine - N | |
| Glycine - G | |
| Serine - S | |
| Glutamine - Q | |
| Tyrosine - Y | |
| Threonine - T | |
| Aspartic acid - D | |
| Glutamic acid - E | |
| SEQ. ID NO: 1 | APCAPRPSCG |
| SEQ. ID NO: 2 | EACVPSVPCP |
| SEQ. ID NO: 3 | ESCGTASGCA |
| SEQ. ID NO: 4 | GLCAGTSACL |
| SEQ. ID NO: 5 | GVCGPSPPCI |
| SEQ. ID NO: 6 | HGCTLPGACN |
| SEQ. ID NO: 7 | HSCTLPGACN |
| SEQ. ID NO: 8 | KDCLQNSLCE |
| SEQ. ID NO: 9 | LPCLPAASCG |
| SEQ. ID NO: 10 | LPCYFTGSCN |
| SEQ. ID NO: 11 | NFCLPSLSCR |
| SEQ. ID NO: 12 | NPCATTNACD |
| SEQ. ID NO: 13 | NPCATTNACE |
| SEQ. ID NO: 14 | NPCATTNACS |
| SEQ. ID NO: 15 | NPCGLRARCG |
| SEQ. ID NO: 16 | NPCGPRSRCG |
| SEQ. ID NO: 17 | NPCSTPASCT |
| SEQ. ID NO: 18 | NPCSTSPSCV |
| SEQ. ID NO: 19 | PACTSSSPCS |
| SEQ. ID NO: 20 | SKCHESTVCP |
| SEQ. ID NO: 21 | SPCVPRTVCV |
| SEQ. ID NO: 22 | SSCSVETACL |
| SEQ. ID NO: 23 | SVCSSGVNCR |
| SEQ. ID NO: 24 | TACPLPGTCH |
| SEQ. ID NO: 25 | TNCSPRPICV |
| SEQ. ID NO: 26 | TSCVPPAPCT |
| SEQ. ID NO: 27 | TTCTSSNTCE |
| SEQ. ID NO: 28 | VPCVPSVPCT |
| SEQ. ID NO: 29 | ATCGPSACIT |
| SEQ. ID NO: 30 | GPCISNPCGL |
| SEQ. ID NO: 31 | GPCLSNPCTS |
| SEQ. ID NO: 32 | GSCVTNPCGP |
| SEQ. ID NO: 33 | LTCFSITCSS |
| SEQ. ID NO: 34 | NPCSTPSCTT |
| SEQ. ID NO: 35 | PSCVTAPCAP |
| SEQ. ID NO: 36 | SDCSSTHCSP |
| SEQ. ID NO: 37 | SLCLPPTCHT |
| SEQ. ID NO: 38 | SLCNLGSCGP |
| SEQ. ID NO: 39 | SPCLVGNCAW |
| SEQ. ID NO: 40 | TACLPGTCAT |
| SEQ. ID NO: 41 | TSCLPALCLP |
| SEQ. ID NO: 42 | TSCSSRPCVP |
| SEQ. ID NO: 43 | TTCGGGSCGV |
| SEQ. ID NO: 44 | VNCRPELCLG |
| SEQ. ID NO: 45 | YVCQPMACLP |
| SEQ. ID NO: 46 | AFSCISACGP |
| SEQ. ID NO: 47 | GSVCSAPCNG |
| SEQ. ID NO: 48 | GVVCGDLCAS |
| SEQ. ID NO: 49 | GVVCGDLCVS |
| SEQ. ID NO: 50 | LTGCLLPCYF |
| SEQ. ID NO: 51 | NEDCKLPCNP |
| SEQ. ID NO: 52 | NFSCVSACGP |
| SEQ. ID NO: 53 | PPTCHTACPL |
| SEQ. ID NO: 54 | PQPCATACKP |
| SEQ. ID NO: 55 | SEDCKLPCNP |
| SEQ. ID NO: 56 | SLGCRTSCSS |
| SEQ. ID NO: 57 | SLSCRTSCSS |
| SEQ. ID NO: 58 | SSSCPLGCTM |
| SEQ. ID NO: 59 | TGSCNSPCLV |
| SEQ. ID NO: 60 | TSSCPLGCTM |
| SEQ. ID NO: 61 | VGSCGSSCRK |
| SEQ. ID NO: 62 | VGVCGGSCKR |
| SEQ. ID NO: 63 | VSNCNWFCEG |
| SEQ. ID NO: 64 | ACGPRPGRCC |
| SEQ. ID NO: 65 | ACGPRPSRCC |
| SEQ. ID NO: 66 | CAPRPSCGPC |
| SEQ. ID NO: 67 | CEPCSAYVIC |
| SEQ. ID NO: 68 | CGLRARCGPC |
| SEQ. ID NO: 69 | CGPRPGRCCI |
| SEQ. ID NO: 70 | CGPRPSRCCI |
| SEQ. ID NO: 71 | CGPRSRCGPC |
| SEQ. ID NO: 72 | CGTSQKGCCN |
| SEQ. ID NO: 73 | CHGCTLPGAC |
| SEQ. ID NO: 74 | CHSCTLPGAC |
| SEQ. ID NO: 75 | CLPCLPAASC |
| SEQ. ID NO: 76 | CLPPTCHTAC |
| SEQ. ID NO: 77 | CLSNPCTSCV |
| SEQ. ID NO: 78 | CLVGNCAWCE |
| SEQ. ID NO: 79 | CNPCSTPASC |
| SEQ. ID NO: 80 | CNPCSTPSCT |
| SEQ. ID NO: 81 | CNPCSTSPSC |
| SEQ. ID NO: 82 | CNSPCLVGNC |
| SEQ. ID NO: 83 | CRTSCSSRPC |
| SEQ. ID NO: 84 | CSLKEHCSAC |
| SEQ. ID NO: 85 | CSPRPICVPC |
| SEQ. ID NO: 86 | CSSTMSYSCC |
| SEQ. ID NO: 87 | CSTPASCTSC |
| SEQ. ID NO: 88 | CSTPSCTTCV |
| SEQ. ID NO: 89 | CTSCVPPAPC |
| SEQ. ID NO: 90 | CTSSNTCEPC |
| SEQ. ID NO: 91 | CVPPAPCTPC |
| SEQ. ID NO: 92 | CVPPSCHGCT |
| SEQ. ID NO: 93 | CVPPSCHSCT |
| SEQ. ID NO: 94 | DCKLPCNPCA |
| SEQ. ID NO: 95 | DCKLPCNPCS |
| SEQ. ID NO: 96 | PCGTSQKGCC |
| SEQ. ID NO: 97 | PCLSNPCTSC |
| SEQ. ID NO: 98 | PCLVGNCAWC |
| SEQ. ID NO: 99 | PCNPCSTPSC |
| SEQ. ID NO: 100 | PCSTPSCTTC |
| SEQ. ID NO: 101 | PCTTCGPTCG |
| SEQ. ID NO: 102 | PCVPPSCHGC |
| SEQ. ID NO: 103 | PCVPPSCHSC |
| SEQ. ID NO: 104 | SCCLPSLGCR |
| SEQ. ID NO: 105 | SCSEELQCCQ |
| SEQ. ID NO: 106 | SCSPCSTTCT |
| SEQ. ID NO: 107 | ASCSTSGTCG |
| SEQ. ID NO: 108 | ASCYIPVGCQ |
| SEQ. ID NO: 109 | ASCYVPVSCQ |
| SEQ. ID NO: 110 | AVCTLPSSCQ |
| SEQ. ID NO: 111 | DLCPTSVSCG |
| SEQ. ID NO: 112 | EICWEPTSCQ |
| SEQ. ID NO: 113 | ETCGEPTSCQ |
| SEQ. ID NO: 114 | ETCNETTSCQ |
| SEQ. ID NO: 115 | ETCWRPNSCQ |
| SEQ. ID NO: 116 | GYCGYRPFCF |
| SEQ. ID NO: 117 | KTCWEPASCQ |
| SEQ. ID NO: 118 | KTCWEPTSCQ |
| SEQ. ID NO: 119 | LDCVDTTPCK |
| SEQ. ID NO: 120 | LGCGYGSFCG |
| SEQ. ID NO: 121 | NSCGYGSGCG |
| SEQ. ID NO: 122 | NYCPSNTMCE |
| SEQ. ID NO: 123 | PACVTSYSCR |
| SEQ. ID NO: 124 | PDCHVEGTCL |
| SEQ. ID NO: 125 | PDCRVEGTCL |
| SEQ. ID NO: 126 | PICSEPSPCS |
| SEQ. ID NO: 127 | PICYIFKPCQ |
| SEQ. ID NO: 128 | PLCYISNSCQ |
| SEQ. ID NO: 129 | PPCGQPTPCS |
| SEQ. ID NO: 130 | PPCHIPQPCV |
| SEQ. ID NO: 131 | PSCGRLASCG |
| SEQ. ID NO: 132 | PSCSESSICQ |
| SEQ. ID NO: 133 | PSCSEVTSCP |
| SEQ. ID NO: 134 | PSCSTSGTCG |
| SEQ. ID NO: 135 | PSCSVSSGCQ |
| SEQ. ID NO: 136 | PSCTESDSCK |
| SEQ. ID NO: 137 | PSCYQTSSCG |
| SEQ. ID NO: 138 | PTCFLLNSCQ |
| SEQ. ID NO: 139 | PTCSVTSSCQ |
| SEQ. ID NO: 140 | PTCWLLNNCH |
| SEQ. ID NO: 141 | PTCYQRTSCV |
| SEQ. ID NO: 142 | PTCYRRTSCV |
| SEQ. ID NO: 143 | PTCYVVKRCP |
| SEQ. ID NO: 144 | PVCFEATICE |
| SEQ. ID NO: 145 | PVCFEATVCE |
| SEQ. ID NO: 146 | PVCSRPASCS |
| SEQ. ID NO: 147 | PVCSWVPACS |
| SEQ. ID NO: 148 | QTCNESSYCL |
| SEQ. ID NO: 149 | QTCWEPTSCQ |
| SEQ. ID NO: 150 | SFCRLGYGCG |
| SEQ. ID NO: 151 | SFCRRGSGCG |
| SEQ. ID NO: 152 | SLCGYGYGCG |
| SEQ. ID NO: 153 | SLCSTEVSCG |
| SEQ. ID NO: 154 | SNCFGQLNCL |
| SEQ. ID NO: 155 | SPCGQPTPCS |
| SEQ. ID NO: 156 | SSCDQSSSCA |
| SEQ. ID NO: 157 | SSCGQSSSCA |
| SEQ. ID NO: 158 | SVCPEPVSCP |
| SEQ. ID NO: 159 | TFCSFDKSCR |
| SEQ. ID NO: 160 | TICSSDKSCR |
| SEQ. ID NO: 161 | TLCVESSPCH |
| SEQ. ID NO: 162 | TPCYQQSSCQ |
| SEQ. ID NO: 163 | VTCSRQTTCI |
| SEQ. ID NO: 164 | YGCGYGSGCG |
| SEQ. ID NO: 165 | YGCGYGSGCR |
| SEQ. ID NO: 166 | YGCIHSTHCG |
| SEQ. ID NO: 167 | AACEPSACQS |
| SEQ. ID NO: 168 | AACEPSPCQS |
| SEQ. ID NO: 169 | AACTMSVCSS |
| SEQ. ID NO: 170 | ADCLGGICLP |
| SEQ. ID NO: 171 | ALCLPSSCHS |
| SEQ. ID NO: 172 | ALCSPSTCQL |
| SEQ. ID NO: 173 | APCLALVCAP |
| SEQ. ID NO: 174 | APCLSLVCTP |
| SEQ. ID NO: 175 | APCLTLVCTP |
| SEQ. ID NO: 176 | APCVALLCRP |
| SEQ. ID NO: 177 | ASCGSLLCRP |
| SEQ. ID NO: 178 | ASCLSFLCRP |
| SEQ. ID NO: 179 | ASCVSLLCRP |
| SEQ. ID NO: 180 | AVCEPSPCQS |
| SEQ. ID NO: 181 | AVCLPVSCQS |
| SEQ. ID NO: 182 | AVCVPVRCQS |
| SEQ. ID NO: 183 | AVCVPVSCQS |
| SEQ. ID NO: 184 | DLCSPSTCQL |
| SEQ. ID NO: 185 | DSCGSSSCGP |
| SEQ. ID NO: 186 | DSCVQSNCFP |
| SEQ. ID NO: 187 | FNCSTRNCSS |
| SEQ. ID NO: 188 | GGCGSYGCSQ |
| SEQ. ID NO: 189 | GSCGFGSCYG |
| SEQ. ID NO: 190 | GSCSSRKCFS |
| SEQ. ID NO: 191 | GVCLPSTCPH |
| SEQ. ID NO: 192 | HSCEGYLCYS |
| SEQ. ID NO: 193 | IVCAAPSCQS |
| SEQ. ID NO: 194 | KTCSTTGCDP |
| SEQ. ID NO: 195 | LACVSQPCQS |
| SEQ. ID NO: 196 | LGCGYGGCGY |
| SEQ. ID NO: 197 | LSCGSRSCSS |
| SEQ. ID NO: 198 | LVCTPVSCVS |
| SEQ. ID NO: 199 | NGCQETYCEP |
| SEQ. ID NO: 200 | NSCRSLSCGS |
| SEQ. ID NO: 201 | PACVISTCPR |
| SEQ. ID NO: 202 | PGCLNQSCGS |
| SEQ. ID NO: 203 | PPCGTAPCLT |
| SEQ. ID NO: 204 | PPCTTALCRP |
| SEQ. ID NO: 205 | PPCYLVSCTP |
| SEQ. ID NO: 206 | PRCTRPICEP |
| SEQ. ID NO: 207 | PSCPVSSCAQ |
| SEQ. ID NO: 208 | PSCQPSVCVP |
| SEQ. ID NO: 209 | PSCSVSNCYQ |
| SEQ. ID NO: 210 | PSCSVSSCAQ |
| SEQ. ID NO: 211 | PSCTSVLCRP |
| SEQ. ID NO: 212 | PTCKSPSCEP |
| SEQ. ID NO: 213 | PTCVISSCPR |
| SEQ. ID NO: 214 | PTCVISTCPR |
| SEQ. ID NO: 215 | PTCYQTICFR |
| SEQ. ID NO: 216 | PVCGGVSCHT |
| SEQ. ID NO: 217 | PVCGRVSCHT |
| SEQ. ID NO: 218 | PVCNKPVCFV |
| SEQ. ID NO: 219 | PVCPTPTCSV |
| SEQ. ID NO: 220 | PVCRSTYCVP |
| SEQ. ID NO: 221 | PVCSKSVCYV |
| SEQ. ID NO: 222 | PVCSRPACYS |
| SEQ. ID NO: 223 | PVCYVPTCSE |
| SEQ. ID NO: 224 | QFCLSKSCQP |
| SEQ. ID NO: 225 | RPCERTACQS |
| SEQ. ID NO: 226 | RSCQTSFCGF |
| SEQ. ID NO: 227 | RSCSSLGCGS |
| SEQ. ID NO: 228 | RSCYSVGCGS |
| SEQ. ID NO: 229 | RVCLPGSCDS |
| SEQ. ID NO: 230 | SFCGFPSCST |
| SEQ. ID NO: 231 | SFCGYPSCST |
| SEQ. ID NO: 232 | SGCDPASCQP |
| SEQ. ID NO: 233 | SGCGGSGCGG |
| SEQ. ID NO: 234 | SGCQPSSCLA |
| SEQ. ID NO: 235 | SHCQPPHCQL |
| SEQ. ID NO: 236 | SICQPATCVA |
| SEQ. ID NO: 237 | SLCVPVSCRP |
| SEQ. ID NO: 238 | SNCLPTSCQP |
| SEQ. ID NO: 239 | SPCLVSSCQP |
| SEQ. ID NO: 240 | SPCQQSSCQE |
| SEQ. ID NO: 241 | SPCQQSYCVP |
| SEQ. ID NO: 242 | SPCSPAVCVS |
| SEQ. ID NO: 243 | SRCQQPSCQP |
| SEQ. ID NO: 244 | SRCYRPHCGQ |
| SEQ. ID NO: 245 | SSCAPIYCRR |
| SEQ. ID NO: 246 | SSCAPVYCRR |
| SEQ. ID NO: 247 | SSCGKGGCGS |
| SEQ. ID NO: 248 | SSCGKRGCGS |
| SEQ. ID NO: 249 | SSCLPVSCRP |
| SEQ. ID NO: 250 | SSCQPAYCTS |
| SEQ. ID NO: 251 | SSCQPSYCRQ |
| SEQ. ID NO: 252 | SSCQPVVCEP |
| SEQ. ID NO: 253 | SSCTAVVCRP |
| SEQ. ID NO: 254 | SSCYQPFCRS |
| SEQ. ID NO: 255 | SSCYRPICGS |
| SEQ. ID NO: 256 | SSCYRPTCGS |
| SEQ. ID NO: 257 | SVCMSGSCQA |
| SEQ. ID NO: 258 | SVCSDQGCDQ |
| SEQ. ID NO: 259 | SVCSDOGCGL |
| SEQ. ID NO: 260 | SVCSDQGCGQ |
| SEQ. ID NO: 261 | SVCSDQGCSQ |
| SEQ. ID NO: 262 | SVCSDQSCGQ |
| SEQ. ID NO: 263 | SVCSHQGCGQ |
| SEQ. ID NO: 264 | SVCSHQGCGR |
| SEQ. ID NO: 265 | SVCVPVSCRP |
| SEQ. ID NO: 266 | SYCRQASCVS |
| SEQ. ID NO: 267 | TACEPSACQS |
| SEQ. ID NO: 268 | TICTASPCQP |
| SEQ. ID NO: 269 | TSCPETSCLP |
| SEQ. ID NO: 270 | TSCQMTNCEQ |
| SEQ. ID NO: 271 | TSCQPVHCET |
| SEQ. ID NO: 272 | TSCQPVLCKS |
| SEQ. ID NO: 273 | TSCQPVLCVP |
| SEQ. ID NO: 274 | TSCVGFVCQP |
| SEQ. ID NO: 275 | TSCVSNPCQV |
| SEQ. ID NO: 276 | TTCFQPTCVS |
| SEQ. ID NO: 277 | TTCFQPTCVT |
| SEQ. ID NO: 278 | TTCFQPTCVY |
| SEQ. ID NO: 279 | TTCISNPCST |
| SEQ. ID NO: 280 | TWCQGSSCQP |
| SEQ. ID NO: 281 | VGCQSSVCVP |
| SEQ. ID NO: 282 | VPCQPSTCVF |
| SEQ. ID NO: 283 | VSCEPSPCQS |
| SEQ. ID NO: 284 | VSCGGPICLP |
| SEQ. ID NO: 285 | VSCKPVLCVA |
| SEQ. ID NO: 286 | VSCPSTSCRP |
| SEQ. ID NO: 287 | VSCQSSVCMP |
| SEQ. ID NO: 288 | VSCTRIVCVA |
| SEQ. ID NO: 289 | VTCEPSPCQS |
| SEQ. ID NO: 290 | VTCQTTVCRP |
| SEQ. ID NO: 291 | YGCGYEGCRY |
| SEQ. ID NO: 292 | AGSCQPSCSE |
| SEQ. ID NO: 293 | ALLCRPLCGV |
| SEQ. ID NO: 294 | ALVCEPVCLR |
| SEQ. ID NO: 295 | ATICEPSCSV |
| SEQ. ID NO: 296 | ATTCEPSCSV |
| SEQ. ID NO: 297 | ATVCEPSCSV |
| SEQ. ID NO: 298 | EGTCLPPCYL |
| SEQ. ID NO: 299 | FSTCRPSCSG |
| SEQ. ID NO: 300 | GFVCQPMCSH |
| SEQ. ID NO: 301 | GLDCGYGCGY |
| SEQ. ID NO: 302 | GLGCGYGCGY |
| SEQ. ID NO: 303 | GLGCSYGCGH |
| SEQ. ID NO: 304 | GLGCSYGCGL |
| SEQ. ID NO: 305 | GSGCGYGCGY |
| SEQ. ID NO: 306 | GTGCGYGCGY |
| SEQ. ID NO: 307 | GVSCHTTCYR |
| SEQ. ID NO: 308 | GYACNFPCSY |
| SEQ. ID NO: 309 | GYGCGYGCGF |
| SEQ. ID NO: 310 | HSPCQASCYV |
| SEQ. ID NO: 311 | HTSCSPACQP |
| SEQ. ID NO: 312 | HTSCSSGCQP |
| SEQ. ID NO: 313 | IRWCHPDCHV |
| SEQ. ID NO: 314 | IRWCRPDCRV |
| SEQ. ID NO: 315 | ISSCGTGCGI |
| SEQ. ID NO: 316 | KGGCGSGCGG |
| SEQ. ID NO: 317 | KGGCGSSCSQ |
| SEQ. ID NO: 318 | LVTCQDSCGS |
| SEQ. ID NO: 319 | LVTCQESCQP |
| SEQ. ID NO: 320 | MSICSSACTD |
| SEQ. ID NO: 321 | MSICSSACTN |
| SEQ. ID NO: 322 | MSVCSSACSD |
| SEQ. ID NO: 323 | PAICEPSCSV |
| SEQ. ID NO: 324 | PASCQKSCYR |
| SEQ. ID NO: 325 | PIYCRRTCYH |
| SEQ. ID NO: 326 | PNSCQTLCVE |
| SEQ. ID NO: 327 | PQPCVPTCFL |
| SEQ. ID NO: 328 | PSACQSGCTS |
| SEQ. ID NO: 329 | PSPCEPSCSE |
| SEQ. ID NO: 330 | PSPCQASCYI |
| SEQ. ID NO: 331 | PSPCQSGCIS |
| SEQ. ID NO: 332 | PSPCQSGCTD |
| SEQ. ID NO: 333 | PSPCQSGCTS |
| SEQ. ID NO: 334 | PTACQPTCYQ |
| SEQ. ID NO: 335 | PTACQPTCYR |
| SEQ. ID NO: 336 | PTPCSTTCRT |
| SEQ. ID NO: 337 | PTSCQKSCYR |
| SEQ. ID NO: 338 | PTSCQPSCES |
| SEQ. ID NO: 339 | PTSCQTSCTL |
| SEQ. ID NO: 340 | PVICEPSCSV |
| SEQ. ID NO: 341 | PVSCVPVCSG |
| SEQ. ID NO: 342 | PVTCVPRCTR |
| SEQ. ID NO: 343 | PVYCRRTCYH |
| SEQ. ID NO: 344 | PVYCRRTCYY |
| SEQ. ID NO: 345 | PVYCVPVCSG |
| SEQ. ID NO: 346 | QPGCESPCEP |
| SEQ. ID NO: 347 | QQSCVSSCRR |
| SEQ. ID NO: 348 | QTSCGSSCGQ |
| SEQ. ID NO: 349 | QTTCHPSCGM |
| SEQ. ID NO: 350 | QTTCRPSCGV |
| SEQ. ID NO: 351 | RGGCGSGCGG |
| SEQ. ID NO: 352 | RLACYSLCSG |
| SEQ. ID NO: 353 | RPACYRPCYS |
| SEQ. ID NO: 354 | RPFCFRRCYS |
| SEQ. ID NO: 355 | RPICRPICSG |
| SEQ. ID NO: 356 | RPLCYRRCYS |
| SEQ. ID NO: 357 | RSPCQASCYV |
| SEQ. ID NO: 358 | RVSCHTTCYR |
| SEQ. ID NO: 359 | SAICRPTCPR |
| SEQ. ID NO: 360 | SDSCKRDCKK |
| SEQ. ID NO: 361 | SEGCGSGCGG |
| SEQ. ID NO: 362 | SFLCRPACSR |
| SEQ. ID NO: 363 | SGGCGSGCGG |
| SEQ. ID NO: 364 | SGGCGSSCGG |
| SEQ. ID NO: 365 | SGSCQAACGQ |
| SEQ. ID NO: 366 | SLLCHPVCKS |
| SEQ. ID NO: 367 | SLLCHPVCRS |
| SEQ. ID NO: 368 | SLLCRPACSP |
| SEQ. ID NO: 369 | SLLCRPACSR |
| SEQ. ID NO: 370 | SLLCRPICRP |
| SEQ. ID NO: 371 | SLLCRPMCSR |
| SEQ. ID NO: 372 | SLLCRPTCSR |
| SEQ. ID NO: 373 | SLLCRPVCQP |
| SEQ. ID NO: 374 | SLLCRPVCRP |
| SEQ. ID NO: 375 | SLLCRPVCRS |
| SEQ. ID NO: 376 | SLLCRPVCSR |
| SEQ. ID NO: 377 | SNPCQVTCSR |
| SEQ. ID NO: 378 | SRGCGSGCGG |
| SEQ. ID NO: 379 | SRSCQSPCYR |
| SEQ. ID NO: 380 | SRSCQSSCYR |
| SEQ. ID NO: 381 | SSGCGYGCGY |
| SEQ. ID NO: 382 | SSGCPMACPG |
| SEQ. ID NO: 383 | SSICQPICSE |
| SEQ. ID NO: 384 | SSPCHTSCYY |
| SEQ. ID NO: 385 | SSPCQPTCYV |
| SEQ. ID NO: 386 | SSPCQQSCYV |
| SEQ. ID NO: 387 | SSPCQTSCYR |
| SEQ. ID NO: 388 | SSSCQQSCRV |
| SEQ. ID NO: 389 | STVCQPACGV |
| SEQ. ID NO: 390 | TDNCQETCGE |
| SEQ. ID NO: 391 | TQPCYEPCLP |
| SEQ. ID NO: 392 | TSSCGTGCGI |
| SEQ. ID NO: 393 | TSSCQPSCGR |
| SEQ. ID NO: 394 | TSSCTTPCYQ |
| SEQ. ID NO: 395 | TSVCLPGCLN |
| SEQ. ID NO: 396 | TTVCLPGCLN |
| SEQ. ID NO: 397 | VANCQAPCST |
| SEQ. ID NO: 398 | VDDCPESCWP |
| SEQ. ID NO: 399 | VKRCPSVCPE |
| SEQ. ID NO: 400 | VSSCQPSCSE |
| SEQ. ID NO: 401 | YEGCRYGCGH |
| SEQ. ID NO: 402 | YGRCRHGCHS |
| SEQ. ID NO: 403 | YGYCRPSCYG |
| SEQ. ID NO: 404 | YRDCQKTCWE |
| SEQ. ID NO: 405 | YRGCQEICWE |
| SEQ. ID NO: 406 | YRGCQETCWR |
| SEQ. ID NO: 407 | YRGCQQTCWE |
| SEQ. ID NO: 408 | YRSCRPSCYG |
| SEQ. ID NO: 409 | GGVCGPSPPC |
| SEQ. ID NO: 410 | GVCGPSPPCI |
| SEQ. ID NO: 411 | VCGPSPPCIT |
| SEQ. ID NO: 412 | CGPSPPCITT |
| SEQ. ID NO: 413 | CAPIYCRRTC |
| SEQ. ID NO: 414 | CAPSPCQASC |
| SEQ. ID NO: 415 | CAPSPCQPAC |
| SEQ. ID NO: 416 | CAPVYCRRTC |
| SEQ. ID NO: 417 | CASSPCQQAC |
| SEQ. ID NO: 418 | CASSSCQPAC |
| SEQ. ID NO: 419 | CASSSCQQSC |
| SEQ. ID NO: 420 | CCGNFSSHSC |
| SEQ. ID NO: 421 | CCGYGGLGCG |
| SEQ. ID NO: 422 | CCNYYGNSCG |
| SEQ. ID NO: 423 | CCNYYRNSCG |
| SEQ. ID NO: 424 | CCSRNFSSCS |
| SEQ. ID NO: 425 | CDAGSCQPSC |
| SEQ. ID NO: 426 | CDPCSLQEGC |
| SEQ. ID NO: 427 | CDPSPCEPSC |
| SEQ. ID NO: 428 | CDPVICEPSC |
| SEQ. ID NO: 429 | CDQGLCQETC |
| SEQ. ID NO: 430 | CEATTCEPSC |
| SEQ. ID NO: 431 | CELPCGTPSC |
| SEQ. ID NO: 432 | CEPAICEPSC |
| SEQ. ID NO: 433 | CEPPCGTAPC |
| SEQ. ID NO: 434 | CEPPCSAPSC |
| SEQ. ID NO: 435 | CEPRSCASSC |
| SEQ. ID NO: 436 | CEPSACQSGC |
| SEQ. ID NO: 437 | CEPSCSVSNC |
| SEQ. ID NO: 438 | CEPSCSVSSC |
| SEQ. ID NO: 439 | CEPSPCQSGC |
| SEQ. ID NO: 440 | CEPTACQPTC |
| SEQ. ID NO: 441 | CEPTSCQTSC |
| SEQ. ID NO: 442 | CEPVCLRPVC |
| SEQ. ID NO: 443 | CETSSCQPRC |
| SEQ. ID NO: 444 | CETTCFQPTC |
| SEQ. ID NO: 445 | CFQPTCVSSC |
| SEQ. ID NO: 446 | CFQPTCVTSC |
| SEQ. ID NO: 447 | CFQPTCVYSC |
| SEQ. ID NO: 448 | CGCGFRRLGC |
| SEQ. ID NO: 449 | CGCGYRGLDC |
| SEQ. ID NO: 450 | CGCNGYYGCY |
| SEQ. ID NO: 451 | CGFGSCYGCG |
| SEQ. ID NO: 452 | CGGSGCGGSC |
| SEQ. ID NO: 453 | CGGSGSSCCV |
| SEQ. ID NO: 454 | CGGVSCHTTC |
| SEQ. ID NO: 455 | CGKGGCGSCG |
| SEQ. ID NO: 456 | CGKRGCGSCG |
| SEQ. ID NO: 457 | CGQDLCQETC |
| SEQ. ID NO: 458 | CGQTSCGSSC |
| SEQ. ID NO: 459 | CGQVLCQETC |
| SEQ. ID NO: 460 | CGRDLCQETC |
| SEQ. ID NO: 461 | CGRVSCHTTC |
| SEQ. ID NO: 462 | CGSCGFGSCY |
| SEQ. ID NO: 463 | CGSCGGSKGC |
| SEQ. ID NO: 464 | CGSGCGVPVC |
| SEQ. ID NO: 465 | CGSLLCRPTC |
| SEQ. ID NO: 466 | CGSRCYVPVC |
| SEQ. ID NO: 467 | CGSSSCGPQC |
| SEQ. ID NO: 468 | CGSVCSDQGC |
| SEQ. ID NO: 469 | CGSVCSDQSC |
| SEQ. ID NO: 470 | CGSVCSHQGC |
| SEQ. ID NO: 471 | CGSYGCSQCS |
| SEQ. ID NO: 472 | CGVCLPSTCP |
| SEQ. ID NO: 473 | CGYEGCRYGC |
| SEQ. ID NO: 474 | CGYGCGYGCG |
| SEQ. ID NO: 475 | CGYGGCGYGC |
| SEQ. ID NO: 476 | CGYGSFCGCG |
| SEQ. ID NO: 477 | CGYGSGCGCG |
| SEQ. ID NO: 478 | CHPSCGMSSC |
| SEQ. ID NO: 479 | CHPSCSISSC |
| SEQ. ID NO: 480 | CHPTCYQTIC |
| SEQ. ID NO: 481 | CHTSCSPACQ |
| SEQ. ID NO: 482 | CHTSCSSGCQ |
| SEQ. ID NO: 483 | CHTTCYRPAC |
| SEQ. ID NO: 484 | CHTTCYRPTC |
| SEQ. ID NO: 485 | CIHSPCQASC |
| SEQ. ID NO: 486 | CIHSTHCGCN |
| SEQ. ID NO: 487 | CIRSPCQASC |
| SEQ. ID NO: 488 | CISSCYRPQC |
| SEQ. ID NO: 489 | CISSPCQQSC |
| SEQ. ID NO: 490 | CKPCSSQSSC |
| SEQ. ID NO: 491 | CKPSCSQSSC |
| SEQ. ID NO: 492 | CKPVCFKPIC |
| SEQ. ID NO: 493 | CKPVCYVPTC |
| SEQ. ID NO: 494 | CKPVSCVPVC |
| SEQ. ID NO: 495 | CKPVYCVPVC |
| SEQ. ID NO: 496 | CKTVYCKPIC |
| SEQ. ID NO: 497 | CLNQSCGSNC |
| SEQ. ID NO: 498 | CLNQSCGSSC |
| SEQ. ID NO: 499 | CLPGCLNQSC |
| SEQ. ID NO: 500 | CLPGSCDSCS |
| SEQ. ID NO: 501 | CLPPCYLVSC |
| SEQ. ID NO: 502 | CLPTSCQPSC |
| SEQ. ID NO: 503 | CLSFLCRPAC |
| SEQ. ID NO: 504 | CLVSSCQPSC |
| SEQ. ID NO: 505 | CMPSPCQPAC |
| SEQ. ID NO: 506 | CMSGSCQAAC |
| SEQ. ID NO: 507 | CNESSYCLPC |
| SEQ. ID NO: 508 | CPASCVSLLC |
| SEQ. ID NO: 509 | CPMACPGSPC |
| SEQ. ID NO: 510 | CPSSCTAVVC |
| SEQ. ID NO: 511 | CPVTCEPSPC |
| SEQ. ID NO: 512 | CQAACEPSAC |
| SEQ. ID NO: 513 | CQAACEPSPC |
| SEQ. ID NO: 514 | CQAACGQSVC |
| SEQ. ID NO: 515 | CQAPCSTKNC |
| SEQ. ID NO: 516 | CQAVCEPSPC |
| SEQ. ID NO: 517 | CQDSCGSSSC |
| SEQ. ID NO: 518 | CQHSSCQPTC |
| SEQ. ID NO: 519 | CQISSCGTGC |
| SEQ. ID NO: 520 | CQKSSCQPAC |
| SEQ. ID NO: 521 | CQPMCSHAAC |
| SEQ. ID NO: 522 | CQPPCTTALC |
| SEQ. ID NO: 523 | CQPSCESSFC |
| SEQ. ID NO: 524 | CQPSCSESTC |
| SEQ. ID NO: 525 | CQPSCTSVLC |
| SEQ. ID NO: 526 | CQPTCGGSSC |
| SEQ. ID NO: 527 | CQPTCSRPSC |
| SEQ. ID NO: 528 | CQPVCPTPTC |
| SEQ. ID NO: 529 | CQPVLCKSSC |
| SEQ. ID NO: 530 | CQPVVCEPSC |
| SEQ. ID NO: 531 | CQQPSCQPAC |
| SEQ. ID NO: 532 | CQQSCRVPVC |
| SEQ. ID NO: 533 | CQQSCYVPVC |
| SEQ. ID NO: 534 | CQQSGCQPAC |
| SEQ. ID NO: 535 | CQQSSCHPAC |
| SEQ. ID NO: 536 | CQQSSCKPAC |
| SEQ. ID NO: 537 | CQQSSCQLAC |
| SEQ. ID NO: 538 | CQQSSCQPAC |
| SEQ. ID NO: 539 | CQQSSCQPTC |
| SEQ. ID NO: 540 | CQQSSCQSAC |
| SEQ. ID NO: 541 | CQQSSCVSCV |
| SEQ. ID NO: 542 | CQQSYCVPVC |
| SEQ. ID NO: 543 | CQSGCISSCT |
| SEQ. ID NO: 544 | CQSGCTDSCT |
| SEQ. ID NO: 545 | CQSGCTSSCT |
| SEQ. ID NO: 546 | CQSSCYRPTC |
| SEQ. ID NO: 547 | CQSVCYQPTC |
| SEQ. ID NO: 548 | CQSVYCQPTC |
| SEQ. ID NO: 549 | CQTACEPSAC |
| SEQ. ID NO: 550 | CQTSSCGTGC |
| SEQ. ID NO: 551 | CQTTCHPSCG |
| SEQ. ID NO: 552 | CQTTCRPSCG |
| SEQ. ID NO: 553 | CQTTCYRTTC |
| SEQ. ID NO: 554 | CQTTRCRTTC |
| SEQ. ID NO: 555 | CQVTCEPSPC |
| SEQ. ID NO: 556 | CRNTSCQPTC |
| SEQ. ID NO: 557 | CRPACSPLAC |
| SEQ. ID NO: 558 | CRPACSRLAC |
| SEQ. ID NO: 559 | CRPACSRPAC |
| SEQ. ID NO: 560 | CRPMCSRPAC |
| SEQ. ID NO: 561 | CRPSCGQTTC |
| SEQ. ID NO: 562 | CRPSCGVSSC |
| SEQ. ID NO: 563 | CRPSCSISSC |
| SEQ. ID NO: 564 | CRPSCSQTTC |
| SEQ. ID NO: 565 | CRPSYCGQSC |
| SEQ. ID NO: 566 | CRPSYCISSC |
| SEQ. ID NO: 567 | CRPSYCQTTC |
| SEQ. ID NO: 568 | CRPTCSRLAC |
| SEQ. ID NO: 569 | CRPTCSSGSC |
| SEQ. ID NO: 570 | CRPTSCQNTC |
| SEQ. ID NO: 571 | CRPVCRSTYC |
| SEQ. ID NO: 572 | CRPVCSRPAC |
| SEQ. ID NO: 573 | CRPVTCVPRC |
| SEQ. ID NO: 574 | CRQSSCQPAC |
| SEQ. ID NO: 575 | CRTTCFHPIC |
| SEQ. ID NO: 576 | CRTTCFQPTC |
| SEQ. ID NO: 577 | CRTTCYRPSC |
| SEQ. ID NO: 578 | CRTTYCRPSC |
| SEQ. ID NO: 579 | CRVTCEPSPC |
| SEQ. ID NO: 580 | CRYGCGHRGC |
| SEQ. ID NO: 581 | CSAPCVALLC |
| SEQ. ID NO: 582 | CSDDSGSCCQ |
| SEQ. ID NO: 583 | CSEDSSSCCQ |
| SEQ. ID NO: 584 | CSEDSYSCCQ |
| SEQ. ID NO: 585 | CSEGCGSGCG |
| SEQ. ID NO: 586 | CSESSPSCCQ |
| SEQ. ID NO: 587 | CSESSSSCCQ |
| SEQ. ID NO: 588 | CSFDKSCRCG |
| SEQ. ID NO: 589 | CSGASSLCCQ |
| SEQ. ID NO: 590 | CSGASSPCCQ |
| SEQ. ID NO: 591 | CSGASSSCCQ |
| SEQ. ID NO: 592 | CSGASTSCCQ |
| SEQ. ID NO: 593 | CSGGCGSGCG |
| SEQ. ID NO: 594 | CSGGCGSSCG |
| SEQ. ID NO: 595 | CSGISSSCCQ |
| SEQ. ID NO: 596 | CSKDSSSCCQ |
| SEQ. ID NO: 597 | CSKGACGSCG |
| SEQ. ID NO: 598 | CSLSCGSRSC |
| SEQ. ID NO: 599 | CSQDLCQETC |
| SEQ. ID NO: 600 | CSRGCGSGCG |
| SEQ. ID NO: 601 | CSRLSSACCG |
| SEQ. ID NO: 602 | CSSCGKGGCG |
| SEQ. ID NO: 603 | CSSCGKRGCG |
| SEQ. ID NO: 604 | CSSDKSCRCG |
| SEQ. ID NO: 605 | CSSGNFSSCC |
| SEQ. ID NO: 606 | CSSSGCGSFC |
| SEQ. ID NO: 607 | CSSSGCGSSC |
| SEQ. ID NO: 608 | CSTPCYQPIC |
| SEQ. ID NO: 609 | CSTTCRTSSC |
| SEQ. ID NO: 610 | CSWVPACSCT |
| SEQ. ID NO: 611 | CTFSPCQQAC |
| SEQ. ID NO: 612 | CTMSVCSSAC |
| SEQ. ID NO: 613 | CTRPICEPCR |
| SEQ. ID NO: 614 | CTSSPCQHAC |
| SEQ. ID NO: 615 | CTSSPCQQAC |
| SEQ. ID NO: 616 | CTSSPCQQSC |
| SEQ. ID NO: 617 | CTSSSCQQAC |
| SEQ. ID NO: 618 | CVALLCRPLC |
| SEQ. ID NO: 619 | CVALVCEPVC |
| SEQ. ID NO: 620 | CVFSSCNTTC |
| SEQ. ID NO: 621 | CVGFVCQPMC |
| SEQ. ID NO: 622 | CVPRCTRPIC |
| SEQ. ID NO: 623 | CVPSPCQVAC |
| SEQ. ID NO: 624 | CVPSRCQASC |
| SEQ. ID NO: 625 | CVPSSCQASC |
| SEQ. ID NO: 626 | CVPVCNKPVC |
| SEQ. ID NO: 627 | CVPVCSKSVC |
| SEQ. ID NO: 628 | CVPVRCKPVC |
| SEQ. ID NO: 629 | CVSLLCRPAC |
| SEQ. ID NO: 630 | CVSLLCRPMC |
| SEQ. ID NO: 631 | CVSLLCRPTC |
| SEQ. ID NO: 632 | CVSLLCRPVC |
| SEQ. ID NO: 633 | CVSNPCQVTC |
| SEQ. ID NO: 634 | CVSRCYRPHC |
| SEQ. ID NO: 635 | CVSSCFRPQC |
| SEQ. ID NO: 636 | CVSSICQPIC |
| SEQ. ID NO: 637 | CVSSPCQPTC |
| SEQ. ID NO: 638 | CVVSCTPPSC |
| SEQ. ID NO: 639 | CVVSCTPPTC |
| SEQ. ID NO: 640 | CYCPKNSIFC |
| SEQ. ID NO: 641 | CYEPCLPRGC |
| SEQ. ID NO: 642 | CYRRCYSSCY |
| SEQ. ID NO: 643 | GCCGYGGLGC |
| SEQ. ID NO: 644 | GCGGCGSGCA |
| SEQ. ID NO: 645 | GCGGCGSGCG |
| SEQ. ID NO: 646 | GCGGCGSSCG |
| SEQ. ID NO: 647 | GCGGCSSSCG |
| SEQ. ID NO: 648 | GCGGSGSSCC |
| SEQ. ID NO: 649 | GCGSGCAGCG |
| SEQ. ID NO: 650 | GCGSGCGGCG |
| SEQ. ID NO: 651 | GCGSGCGGCS |
| SEQ. ID NO: 652 | GCGSSCGGCD |
| SEQ. ID NO: 653 | GCGSSCGGCG |
| SEQ. ID NO: 654 | GCGSSCSQCS |
| SEQ. ID NO: 655 | GCGYSSSCCG |
| SEQ. ID NO: 656 | GCKGGCGSCG |
| SEQ. ID NO: 657 | GCSGCSGGCG |
| SEQ. ID NO: 658 | ICSGASSLCC |
| SEQ. ID NO: 659 | ICSGASSPCC |
| SEQ. ID NO: 660 | MCCNYYGNSC |
| SEQ. ID NO: 661 | MCCNYYRNSC |
| SEQ. ID NO: 662 | MCYGYGCGCG |
| SEQ. ID NO: 663 | NCCSRNFSSC |
| SEQ. ID NO: 664 | PCSLQEGCCR |
| SEQ. ID NO: 665 | PCSSQSSCCV |
| SEQ. ID NO: 666 | SCCAPASSCQ |
| SEQ. ID NO: 667 | SCCAPASTCQ |
| SEQ. ID NO: 668 | SCCAPTSSCQ |
| SEQ. ID NO: 669 | SCCGYRPLCY |
| SEQ. ID NO: 670 | SCCVPASSCQ |
| SEQ. ID NO: 671 | SCCVPTSSCQ |
| SEQ. ID NO: 672 | SCGCSKGACG |
| SEQ. ID NO: 673 | SCGGCDSSCG |
| SEQ. ID NO: 674 | SCGGCGSGCG |
| SEQ. ID NO: 675 | SCGGCGSSCG |
| SEQ. ID NO: 676 | SCGGCKGGCG |
| SEQ. ID NO: 677 | SCGGSKGCCG |
| SEQ. ID NO: 678 | SCGSGCRGCG |
| SEQ. ID NO: 679 | SCYGCGYGCI |
| SEQ. ID NO: 680 | TCCVPVPSCG |
| SEQ. ID NO: 681 | TCSDDSGSCC |
| SEQ. ID NO: 682 | TCSEDSSSCC |
| SEQ. ID NO: 683 | TCSEDSYSCC |
| SEQ. ID NO: 684 | TCSESSPSCC |
| SEQ. ID NO: 685 | TCSESSSSCC |
| SEQ. ID NO: 686 | TCSKDSSSCC |
| SEQ. ID NO: 687 | TCSRLSSACC |
| SEQ. ID NO: 688 | VCCQPTPICD |
| SEQ. ID NO: 689 | VCSEDSSSCC |
| SEQ. ID NO: 690 | VCSGASSLCC |
| SEQ. ID NO: 691 | VCSGASSPCC |
| SEQ. ID NO: 692 | VCSGASSSCC |
| SEQ. ID NO: 693 | VCSGASTSCC |
| SEQ. ID NO: 694 | VCSGDSSCCQ |
| SEQ. ID NO: 695 | VCSGISSSCC |
| SEQ. ID NO: 696 | YCVPIPSCCA |
| SEQ. ID NO: 697 | CASSCCTPSC |
| SEQ. ID NO: 698 | CCDNCPPPCH |
| SEQ. ID NO: 699 | CCEPCLPRGC |
| SEQ. ID NO: 700 | CCGAASSCCR |
| SEQ. ID NO: 701 | CCGCGGSGCG |
| SEQ. ID NO: 702 | CCGPSSSCCQ |
| SEQ. ID NO: 703 | CCGSGCGGCG |
| SEQ. ID NO: 704 | CCKPYCSQCS |
| SEQ. ID NO: 705 | CCMPVSSCCA |
| SEQ. ID NO: 706 | CCNYYRNCCG |
| SEQ. ID NO: 707 | CCPSCVVSSC |
| SEQ. ID NO: 708 | CCPSYCVSSC |
| SEQ. ID NO: 709 | CCQPICGSSC |
| SEQ. ID NO: 710 | CCQPICVTSC |
| SEQ. ID NO: 711 | CCQPTCLSSC |
| SEQ. ID NO: 712 | CCQPTCLTSC |
| SEQ. ID NO: 713 | CCQPTCVASC |
| SEQ. ID NO: 714 | CCQPTCVTSC |
| SEQ. ID NO: 715 | CCQPYCHPTC |
| SEQ. ID NO: 716 | CCQQSSCVSC |
| SEQ. ID NO: 717 | CCQSSCFKPC |
| SEQ. ID NO: 718 | CCQSSCSKPC |
| SEQ. ID NO: 719 | CCQSSCYKPC |
| SEQ. ID NO: 720 | CCQTICRSTC |
| SEQ. ID NO: 721 | CCQTTCHPSC |
| SEQ. ID NO: 722 | CCQTTCRPSC |
| SEQ. ID NO: 723 | CCRVPTCSCS |
| SEQ. ID NO: 724 | CCSPGCQPTC |
| SEQ. ID NO: 725 | CCSSGCGSSC |
| SEQ. ID NO: 726 | CCSSSCGSCG |
| SEQ. ID NO: 727 | CCTQEQNCCE |
| SEQ. ID NO: 728 | CCVPIPSCCA |
| SEQ. ID NO: 729 | CCVPISSCCA |
| SEQ. ID NO: 730 | CCVPVCYQCK |
| SEQ. ID NO: 731 | CCVPVPSCCA |
| SEQ. ID NO: 732 | CCVPVPSCCV |
| SEQ. ID NO: 733 | CCVPVSSCCA |
| SEQ. ID NO: 734 | CDSSCCQPSC |
| SEQ. ID NO: 735 | CDTCPPPCCK |
| SEQ. ID NO: 736 | CEPCRRPVCC |
| SEQ. ID NO: 737 | CEPSCCQPVC |
| SEQ. ID NO: 738 | CEPSCCSAVC |
| SEQ. ID NO: 739 | CETSCCQPSC |
| SEQ. ID NO: 740 | CETTCCRTTC |
| SEQ. ID NO: 741 | CFSGCGSSCC |
| SEQ. ID NO: 742 | CGCSQSNCCK |
| SEQ. ID NO: 743 | CGCSQSSCCK |
| SEQ. ID NO: 744 | CGGCGGCGGC |
| SEQ. ID NO: 745 | CGGCGGGCCG |
| SEQ. ID NO: 746 | CGGCGSGCCV |
| SEQ. ID NO: 747 | CGGCGSSCCV |
| SEQ. ID NO: 748 | CGGGCCGSSC |
| SEQ. ID NO: 749 | CGGSCCGSSC |
| SEQ. ID NO: 750 | CGQSCCRPAC |
| SEQ. ID NO: 751 | CGQSCCRPVC |
| SEQ. ID NO: 752 | CGSCGCSQCN |
| SEQ. ID NO: 753 | CGSCGCSQCS |
| SEQ. ID NO: 754 | CGSFCCQSSC |
| SEQ. ID NO: 755 | CGSGCCVPVC |
| SEQ. ID NO: 756 | CGSSCCGSGC |
| SEQ. ID NO: 757 | CGSSCCQPCY |
| SEQ. ID NO: 758 | CGSSCCQPIC |
| SEQ. ID NO: 759 | CGSSCCQPSC |
| SEQ. ID NO: 760 | CGSSCCQSSC |
| SEQ. ID NO: 761 | CGSSCCVPIC |
| SEQ. ID NO: 762 | CGSSCCVPVC |
| SEQ. ID NO: 763 | CGSSCSQCSC |
| SEQ. ID NO: 764 | CGYGSCCGCG |
| SEQ. ID NO: 765 | CHPRCCISSC |
| SEQ. ID NO: 766 | CHPSCCESSC |
| SEQ. ID NO: 767 | CHPSCCISSC |
| SEQ. ID NO: 768 | CHPTCCQNTC |
| SEQ. ID NO: 769 | CHPTCCQTIC |
| SEQ. ID NO: 770 | CHPVCCQTTC |
| SEQ. ID NO: 771 | CHPVCKSTCC |
| SEQ. ID NO: 772 | CHPVCRSTCC |
| SEQ. ID NO: 773 | CISSCCHPSC |
| SEQ. ID NO: 774 | CISSCCKPSC |
| SEQ. ID NO: 775 | CISSCCRPSC |
| SEQ. ID NO: 776 | CISSCTPSCC |
| SEQ. ID NO: 777 | CISSSCCPSC |
| SEQ. ID NO: 778 | CKAVCCVPTC |
| SEQ. ID NO: 779 | CKPCCSQASC |
| SEQ. ID NO: 780 | CKPCCSQSRC |
| SEQ. ID NO: 781 | CKPCCSQSSC |
| SEQ. ID NO: 782 | CKPCCSSSGC |
| SEQ. ID NO: 783 | CKPCSCFSGC |
| SEQ. ID NO: 784 | CKPCSCSSGC |
| SEQ. ID NO: 785 | CKPCYCSSGC |
| SEQ. ID NO: 786 | CKPICCVPVC |
| SEQ. ID NO: 787 | CKPQCCQSVC |
| SEQ. ID NO: 788 | CKPSCCQTTC |
| SEQ. ID NO: 789 | CKPVCCAPTC |
| SEQ. ID NO: 790 | CKPVCCKPIC |
| SEQ. ID NO: 791 | CKPVCCKSIC |
| SEQ. ID NO: 792 | CKPVCCLPTC |
| SEQ. ID NO: 793 | CKPVCCVPTC |
| SEQ. ID NO: 794 | CKPVCCVPVC |
| SEQ. ID NO: 795 | CKPVCCVSTC |
| SEQ. ID NO: 796 | CKPYCCQSSC |
| SEQ. ID NO: 797 | CKPYCSQCSC |
| SEQ. ID NO: 798 | CKSNCCKPVC |
| SEQ. ID NO: 799 | CKTVCCKPVC |
| SEQ. ID NO: 800 | CLPPCCVVSC |
| SEQ. ID NO: 801 | CLTSCCQPSC |
| SEQ. ID NO: 802 | CNPCCSQSSC |
| SEQ. ID NO: 803 | CPESCCELPC |
| SEQ. ID NO: 804 | CPESCCEPHC |
| SEQ. ID NO: 805 | CPESCCEPPC |
| SEQ. ID NO: 806 | CPFSCPTTCC |
| SEQ. ID NO: 807 | CPGDCFTCCT |
| SEQ. ID NO: 808 | CPSCVVSSCC |
| SEQ. ID NO: 809 | CPSYCVSSCC |
| SEQ. ID NO: 810 | CPTTCCRTTC |
| SEQ. ID NO: 811 | CQETCCRPSC |
| SEQ. ID NO: 812 | CQHACCVPVC |
| SEQ. ID NO: 813 | CQNTCCRTTC |
| SEQ. ID NO: 814 | CQPACCQPTC |
| SEQ. ID NO: 815 | CQPACCTASC |
| SEQ. ID NO: 816 | CQPACCTSSC |
| SEQ. ID NO: 817 | CQPACCTTSC |
| SEQ. ID NO: 818 | CQPACCVPVC |
| SEQ. ID NO: 819 | CQPACCVSSC |
| SEQ. ID NO: 820 | CQPCCHPTCY |
| SEQ. ID NO: 821 | CQPCCRPTSC |
| SEQ. ID NO: 822 | CQPICCGSSC |
| SEQ. ID NO: 823 | CQPICGSSCC |
| SEQ. ID NO: 824 | CQPICVTSCC |
| SEQ. ID NO: 825 | CQPNCCRPSC |
| SEQ. ID NO: 826 | CQPRCCETSC |
| SEQ. ID NO: 827 | CQPSCCRPAC |
| SEQ. ID NO: 828 | CQPSCCSTPC |
| SEQ. ID NO: 829 | CQPSCCSTTC |
| SEQ. ID NO: 830 | CQPSCCVPSC |
| SEQ. ID NO: 831 | CQPSCCVSSC |
| SEQ. ID NO: 832 | CQPTCCGSSC |
| SEQ. ID NO: 833 | CQPTCCHPSC |
| SEQ. ID NO: 834 | CQPTCCQPTC |
| SEQ. ID NO: 835 | CQPTCCRPRC |
| SEQ. ID NO: 836 | CQPTCCRPSC |
| SEQ. ID NO: 837 | CQPTCCRTTC |
| SEQ. ID NO: 838 | CQPTCLSSCC |
| SEQ. ID NO: 839 | CQPTCLTSCC |
| SEQ. ID NO: 840 | CQPTCVASCC |
| SEQ. ID NO: 841 | CQPTCVTSCC |
| SEQ. ID NO: 842 | CQPVCCQPTC |
| SEQ. ID NO: 843 | CQPYCHPTCC |
| SEQ. ID NO: 844 | CQQACCMPVC |
| SEQ. ID NO: 845 | CQQACCVPIC |
| SEQ. ID NO: 846 | CQQACCVPVC |
| SEQ. ID NO: 847 | CQQSCCVPVC |
| SEQ. ID NO: 848 | CQQSCCVSVC |
| SEQ. ID NO: 849 | CQSMCCQPTC |
| SEQ. ID NO: 850 | CQSNCCVPVC |
| SEQ. ID NO: 851 | CQSSCCKPCS |
| SEQ. ID NO: 852 | CQSSCCQSSC |
| SEQ. ID NO: 853 | CQSSCCVPVC |
| SEQ. ID NO: 854 | CQSSCFKPCC |
| SEQ. ID NO: 855 | CQSSCSKPCC |
| SEQ. ID NO: 856 | CQSVCCQPTC |
| SEQ. ID NO: 857 | CQTICRSTCC |
| SEQ. ID NO: 858 | CQTTCCRPSC |
| SEQ. ID NO: 859 | CQTTCCRTTC |
| SEQ. ID NO: 860 | CRATCCRPSC |
| SEQ. ID NO: 861 | CRGCGPSCCA |
| SEQ. ID NO: 862 | CRPACCETTC |
| SEQ. ID NO: 863 | CRPACCONTC |
| SEQ. ID NO: 864 | CRPCCWATTC |
| SEQ. ID NO: 865 | CRPICRPACC |
| SEQ. ID NO: 866 | CRPLCCQTTC |
| SEQ. ID NO: 867 | CRPQCCQSVC |
| SEQ. ID NO: 868 | CRPQCCQTTC |
| SEQ. ID NO: 869 | CRPRCCISSC |
| SEQ. ID NO: 870 | CRPSCCESSC |
| SEQ. ID NO: 871 | CRPSCCETTC |
| SEQ. ID NO: 872 | CRPSCCISSC |
| SEQ. ID NO: 873 | CRPSCCKPQC |
| SEQ. ID NO: 874 | CRPSCCMSSC |
| SEQ. ID NO: 875 | CRPSCCQTTC |
| SEQ. ID NO: 876 | CRPSCCRPSC |
| SEQ. ID NO: 877 | CRPSCCVSRC |
| SEQ. ID NO: 878 | CRPSCCVSSC |
| SEQ. ID NO: 879 | CRPTCCETTC |
| SEQ. ID NO: 880 | CRPTCCQNTC |
| SEQ. ID NO: 881 | CRPTCCQTTC |
| SEQ. ID NO: 882 | CRPVCCDPCS |
| SEQ. ID NO: 883 | CRPVCCQTTC |
| SEQ. ID NO: 884 | CRPVCQPACC |
| SEQ. ID NO: 885 | CRPVCRPACC |
| SEQ. ID NO: 886 | CRPVCRPTCC |
| SEQ. ID NO: 887 | CRPVCRSTCC |
| SEQ. ID NO: 888 | CRPYCCESSC |
| SEQ. ID NO: 889 | CRRPVCCDPC |
| SEQ. ID NO: 890 | CRSQCCQSVC |
| SEQ. ID NO: 891 | CRTTCCHPSC |
| SEQ. ID NO: 892 | CRTTCCQPIC |
| SEQ. ID NO: 893 | CRTTCCQPTC |
| SEQ. ID NO: 894 | CRTTCCRPSC |
| SEQ. ID NO: 895 | CRTTCCRTTC |
| SEQ. ID NO: 896 | CSCSSCGSCA |
| SEQ. ID NO: 897 | CSCSSCGSCG |
| SEQ. ID NO: 898 | CSCTSCGSCG |
| SEQ. ID NO: 899 | CSPACQPTCC |
| SEQ. ID NO: 900 | CSPGCQPTCC |
| SEQ. ID NO: 901 | CSPSCCQTTC |
| SEQ. ID NO: 902 | CSQCSCYKPC |
| SEQ. ID NO: 903 | CSQSNCCKPC |
| SEQ. ID NO: 904 | CSQSSCCKPC |
| SEQ. ID NO: 905 | CSSGCGSCCQ |
| SEQ. ID NO: 906 | CSSGCGSSCC |
| SEQ. ID NO: 907 | CSSGCQPACC |
| SEQ. ID NO: 908 | CSSSCCQPSC |
| SEQ. ID NO: 909 | CSTPCCQPTC |
| SEQ. ID NO: 910 | CSTTCCQPIC |
| SEQ. ID NO: 911 | CTAVVCRPCC |
| SEQ. ID NO: 912 | CTDSCTPSCC |
| SEQ. ID NO: 913 | CTPSCCQPAC |
| SEQ. ID NO: 914 | CTRPICEPCC |
| SEQ. ID NO: 915 | CTSSCTPSCC |
| SEQ. ID NO: 916 | CVPACSCSSC |
| SEQ. ID NO: 917 | CVPACSCTSC |
| SEQ. ID NO: 918 | CVPVCCKPVC |
| SEQ. ID NO: 919 | CVPVCCVPTC |
| SEQ. ID NO: 920 | CVPVCCVPVC |
| SEQ. ID NO: 921 | CVSCVSSPCC |
| SEQ. ID NO: 922 | CVSRCCRPQC |
| SEQ. ID NO: 923 | CVSSCCKPQC |
| SEQ. ID NO: 924 | CVSSCCQHSC |
| SEQ. ID NO: 925 | CVSSCCQPFC |
| SEQ. ID NO: 926 | CVSSCCQPSC |
| SEQ. ID NO: 927 | CVSSCCRPQC |
| SEQ. ID NO: 928 | CVSTCCRPTC |
| SEQ. ID NO: 929 | CVTRCCSTPC |
| SEQ. ID NO: 930 | CVTSCCQPAC |
| SEQ. ID NO: 931 | CVTSCCQPSC |
| SEQ. ID NO: 932 | CVYSCCQPFC |
| SEQ. ID NO: 933 | CVYSCCQPSC |
| SEQ. ID NO: 934 | GCCGCSEGCG |
| SEQ. ID NO: 935 | GCCGCSGGCG |
| SEQ. ID NO: 936 | GCCGCSRGCG |
| SEQ. ID NO: 937 | GCCRPITCCP |
| SEQ. ID NO: 938 | GCGSSCCQCS |
| SEQ. ID NO: 939 | GCGVPVCCCS |
| SEQ. ID NO: 940 | LCCPCQTTCS |
| SEQ. ID NO: 941 | PCCCLRPVCG |
| SEQ. ID NO: 942 | PCCCRPVTCQ |
| SEQ. ID NO: 943 | PCCCVRPVCG |
| SEQ. ID NO: 944 | PCCSQASCCV |
| SEQ. ID NO: 945 | PCCSQSRCCV |
| SEQ. ID NO: 946 | PCCSQSSCCK |
| SEQ. ID NO: 947 | PCCSQSSCCV |
| SEQ. ID NO: 948 | PCCWATTCCQ |
| SEQ. ID NO: 949 | QCSCCKPYCS |
| SEQ. ID NO: 950 | RCYVPVCCCK |
| SEQ. ID NO: 951 | SCCAPVYCCK |
| SEQ. ID NO: 952 | SCCISSSCCP |
| SEQ. ID NO: 953 | SCCVSSCRCP |
| SEQ. ID NO: 954 | SCGCSQCSCY |
| SEQ. ID NO: 955 | SCGLENCCCP |
| SEQ. ID NO: 956 | VCCGASSCCQ |
| SEQ. ID NO: 957 | VCCGDSSCCQ |
| SEQ. ID NO: 958 | CASSCCTPSCC |
| SEQ. ID NO: 959 | CCCPSCVVSSC |
| SEQ. ID NO: 960 | CCCPSYCVSSC |
| SEQ. ID NO: 961 | CCCSSGCGSSC |
| SEQ. ID NO: 962 | CCDTCPPPCCK |
| SEQ. ID NO: 963 | CCEPHCCALSC |
| SEQ. ID NO: 964 | CCEPPCCAPSC |
| SEQ. ID NO: 965 | CCEPPCCATSC |
| SEQ. ID NO: 966 | CCETSCCQPSC |
| SEQ. ID NO: 967 | CCGSSCCGSGC |
| SEQ. ID NO: 968 | CCGSSCCGSSC |
| SEQ. ID NO: 969 | CCHPRCCISSC |
| SEQ. ID NO: 970 | CCHPSCCESSC |
| SEQ. ID NO: 971 | CCHPSCCISSC |
| SEQ. ID NO: 972 | CCHPSCCVSSC |
| SEQ. ID NO: 973 | CCHPTCCQNTC |
| SEQ. ID NO: 974 | CCHPTCCQTIC |
| SEQ. ID NO: 975 | CCISSCCKPSC |
| SEQ. ID NO: 976 | CCISSCCRPSC |
| SEQ. ID NO: 977 | CCISSSCCPSC |
| SEQ. ID NO: 978 | CCKAVCCVPTC |
| SEQ. ID NO: 979 | CCKPCCSQASC |
| SEQ. ID NO: 980 | CCKPCCSQSRC |
| SEQ. ID NO: 981 | CCKPCCSQSSC |
| SEQ. ID NO: 982 | CCKPCCSSSGC |
| SEQ. ID NO: 983 | CCKPCSCFSGC |
| SEQ. ID NO: 984 | CCKPCSCSSGC |
| SEQ. ID NO: 985 | CCKPCYCSSGC |
| SEQ. ID NO: 986 | CCKPICCVPVC |
| SEQ. ID NO: 987 | CCKPQCCQSVC |
| SEQ. ID NO: 988 | CCKPVCCKPIC |
| SEQ. ID NO: 989 | CCKPYCCQSSC |
| SEQ. ID NO: 990 | CCKPYCSQCSC |
| SEQ. ID NO: 991 | CCMPVCCKPVC |
| SEQ. ID NO: 992 | CCMPVCCKTVC |
| SEQ. ID NO: 993 | CCMSSCCKPQC |
| SEQ. ID NO: 994 | CCNPCCSQSSC |
| SEQ. ID NO: 995 | CCPGDCFTCCT |
| SEQ. ID NO: 996 | CCPSCVVSSCC |
| SEQ. ID NO: 997 | CCPSYCVSSCC |
| SEQ. ID NO: 998 | CCQNTCCRTTC |
| SEQ. ID NO: 999 | CCQPACCVSSC |
| SEQ. ID NO: 1000 | CCQPCCHPTCY |
| SEQ. ID NO: 1001 | CCQPCCRPTSC |
| SEQ. ID NO: 1002 | CCQPICGSSCC |
| SEQ. ID NO: 1003 | CCQPICVTSCC |
| SEQ. ID NO: 1004 | CCQPNCCRPSC |
| SEQ. ID NO: 1005 | CCQPSCCETSC |
| SEQ. ID NO: 1006 | CCQPSCCRPAC |
| SEQ. ID NO: 1007 | CCQPSCCSTPC |
| SEQ. ID NO: 1008 | CCQPSCCSTTC |
| SEQ. ID NO: 1009 | CCQPSCCVPSC |
| SEQ. ID NO: 1010 | CCQPSCCVSSC |
| SEQ. ID NO: 1011 | CCQPTCCHPSC |
| SEQ. ID NO: 1012 | CCQPTCCQPTC |
| SEQ. ID NO: 1013 | CCQPTCCRPRC |
| SEQ. ID NO: 1014 | CCQPTCCRPSC |
| SEQ. ID NO: 1015 | CCQPTCCRPTC |
| SEQ. ID NO: 1016 | CCQPTCCRTTC |
| SEQ. ID NO: 1017 | CCQPTCLSSCC |
| SEQ. ID NO: 1018 | CCQPTCLTSCC |
| SEQ. ID NO: 1019 | CCQPTCVASCC |
| SEQ. ID NO: 1020 | CCQPTCVTSCC |
| SEQ. ID NO: 1021 | CCQPYCHPTCC |
| SEQ. ID NO: 1022 | CCQSMCCQPTC |
| SEQ. ID NO: 1023 | CCQSNCCVPVC |
| SEQ. ID NO: 1024 | CCQSSCCKPCS |
| SEQ. ID NO: 1025 | CCQSSCCKPSC |
| SEQ. ID NO: 1026 | CCQSSCCKPYC |
| SEQ. ID NO: 1027 | CCQSSCCQSSC |
| SEQ. ID NO: 1028 | CCQSSCCVPVC |
| SEQ. ID NO: 1029 | CCQSSCFKPCC |
| SEQ. ID NO: 1030 | CCQSSCSKPCC |
| SEQ. ID NO: 1031 | CCQSSCYKPCC |
| SEQ. ID NO: 1032 | CCQSVCCQPTC |
| SEQ. ID NO: 1033 | CCQTICRSTCC |
| SEQ. ID NO: 1034 | CCQTTCCRPSC |
| SEQ. ID NO: 1035 | CCQTTCCRTTC |
| SEQ. ID NO: 1036 | CCRPACCETTC |
| SEQ. ID NO: 1037 | CCRPACCQNTC |
| SEQ. ID NO: 1038 | CCRPLCCQTTC |
| SEQ. ID NO: 1039 | CCRPQCCQSVC |
| SEQ. ID NO: 1040 | CCRPQCCQTTC |
| SEQ. ID NO: 1041 | CCRPSCCESSC |
| SEQ. ID NO: 1042 | CCRPSCCETTC |
| SEQ. ID NO: 1043 | CCRPSCCGSSC |
| SEQ. ID NO: 1044 | CCRPSCCISSC |
| SEQ. ID NO: 1045 | CCRPSCCKPQC |
| SEQ. ID NO: 1046 | CCRPSCCQTTC |
| SEQ. ID NO: 1047 | CCRPSCCVSRC |
| SEQ. ID NO: 1048 | CCRPSCCVSSC |
| SEQ. ID NO: 1049 | CCRPTCCQNTC |
| SEQ. ID NO: 1050 | CCRPTCCQTTC |
| SEQ. ID NO: 1051 | CCRPVCCDPCS |
| SEQ. ID NO: 1052 | CCRTTCCQPTC |
| SEQ. ID NO: 1053 | CCRTTCCRPSC |
| SEQ. ID NO: 1054 | CCRTTCCRTTC |
| SEQ. ID NO: 1055 | CCSCSSCGSCA |
| SEQ. ID NO: 1056 | CCSPGCQPTCC |
| SEQ. ID NO: 1057 | CCSQSSCCKPC |
| SEQ. ID NO: 1058 | CCSSGCGSCCQ |
| SEQ. ID NO: 1059 | CCSSGCGSSCC |
| SEQ. ID NO: 1060 | CCSTPCCQPTC |
| SEQ. ID NO: 1061 | CCVPACSCSSC |
| SEQ. ID NO: 1062 | CCVPACSCTSC |
| SEQ. ID NO: 1063 | CCVPICCKPIC |
| SEQ. ID NO: 1064 | CCVPICCKPVC |
| SEQ. ID NO: 1065 | CCVPVCCKPIC |
| SEQ. ID NO: 1066 | CCVPVCCKPVC |
| SEQ. ID NO: 1067 | CCVPVCCKSNC |
| SEQ. ID NO: 1068 | CCVPVCCKTVC |
| SEQ. ID NO: 1069 | CCVPVCCSSSC |
| SEQ. ID NO: 1070 | CCVPVCCVPVC |
| SEQ. ID NO: 1071 | CCVSSCCKPQC |
| SEQ. ID NO: 1072 | CCVSSCCQHSC |
| SEQ. ID NO: 1073 | CCVSSCCQPSC |
| SEQ. ID NO: 1074 | CCVSSCCRPQC |
| SEQ. ID NO: 1075 | CCVSTCCRPTC |
| SEQ. ID NO: 1076 | CCVSVCCKPVC |
| SEQ. ID NO: 1077 | CDSSCCQPSCC |
| SEQ. ID NO: 1078 | CEPCCRPVCCD |
| SEQ. ID NO: 1079 | CFKPCCCQSSC |
| SEQ. ID NO: 1080 | CGDGCCCPSCY |
| SEQ. ID NO: 1081 | CGGGCCGSSCC |
| SEQ. ID NO: 1082 | CGGSCCGSSCC |
| SEQ. ID NO: 1083 | CGLENCCCPSC |
| SEQ. ID NO: 1084 | CGQSCCRPACC |
| SEQ. ID NO: 1085 | CGQSCCRPVCC |
| SEQ. ID NO: 1086 | CGSCCQSSCCN |
| SEQ. ID NO: 1087 | CGSCGCSQCNC |
| SEQ. ID NO: 1088 | CGSCGCSQCSC |
| SEQ. ID NO: 1089 | CGSGCCGPVCC |
| SEQ. ID NO: 1090 | CGSGCCVPVCC |
| SEQ. ID NO: 1091 | CGSNCCQPCCR |
| SEQ. ID NO: 1092 | CGSSCCQPCCH |
| SEQ. ID NO: 1093 | CGSSCCQPCCR |
| SEQ. ID NO: 1094 | CGSSCCQPCYC |
| SEQ. ID NO: 1095 | CGSSCCQPSCC |
| SEQ. ID NO: 1096 | CGSSCCQSSCC |
| SEQ. ID NO: 1097 | CGSSCCVPICC |
| SEQ. ID NO: 1098 | CGSSCCVPVCC |
| SEQ. ID NO: 1099 | CGSSCSQCSCC |
| SEQ. ID NO: 1100 | CGVPVCCCSCS |
| SEQ. ID NO: 1101 | CHPRCCISSCC |
| SEQ. ID NO: 1102 | CHPSCCESSCC |
| SEQ. ID NO: 1103 | CHPSCCISSCC |
| SEQ. ID NO: 1104 | CHPTCCQNTCC |
| SEQ. ID NO: 1105 | CISSCCHPSCC |
| SEQ. ID NO: 1106 | CISSCCKPSCC |
| SEQ. ID NO: 1107 | CISSCCRPSCC |
| SEQ. ID NO: 1108 | CISSSCCPSCC |
| SEQ. ID NO: 1109 | CKPCCCSSGCG |
| SEQ. ID NO: 1110 | CKPCCSQASCC |
| SEQ. ID NO: 1111 | CKPCCSQSRCC |
| SEQ. ID NO: 1112 | CKPCCSQSSCC |
| SEQ. ID NO: 1113 | CKPQCCQSMCC |
| SEQ. ID NO: 1114 | CKPQCCQSVCC |
| SEQ. ID NO: 1115 | CKPVCCCVPAC |
| SEQ. ID NO: 1116 | CKPVCCKPICC |
| SEQ. ID NO: 1117 | CKPVCCMPVCC |
| SEQ. ID NO: 1118 | CKPVCCVPVCC |
| SEQ. ID NO: 1119 | CKPVCCVSVCC |
| SEQ. ID NO: 1120 | CKPYCSQCSCC |
| SEQ. ID NO: 1121 | CLPCCRPTCCQ |
| SEQ. ID NO: 1122 | CLTSCCQPSCC |
| SEQ. ID NO: 1123 | CMSSCCKPQCC |
| SEQ. ID NO: 1124 | CNPCCSQSSCC |
| SEQ. ID NO: 1125 | CPACCVSSCCQ |
| SEQ. ID NO: 1126 | CPESCCEPHCC |
| SEQ. ID NO: 1127 | CPESCCEPPCC |
| SEQ. ID NO: 1128 | CPSCCESSCCR |
| SEQ. ID NO: 1129 | CPSCCQTTCCR |
| SEQ. ID NO: 1130 | CPSCCVSSCCR |
| SEQ. ID NO: 1131 | CQCSCCKPYCS |
| SEQ. ID NO: 1132 | CQETCCRPSCC |
| SEQ. ID NO: 1133 | CQNTCCRTTCC |
| SEQ. ID NO: 1134 | CQPACCTASCC |
| SEQ. ID NO: 1135 | CQPACCTSSCC |
| SEQ. ID NO: 1136 | CQPACCTTSCC |
| SEQ. ID NO: 1137 | CQPACCVPVCC |
| SEQ. ID NO: 1138 | CQPACCVSSCC |
| SEQ. ID NO: 1139 | CQPCCHPTCCQ |
| SEQ. ID NO: 1140 | CQPCCRPACCE |
| SEQ. ID NO: 1141 | CQPCCRPACCQ |
| SEQ. ID NO: 1142 | CQPCCRPTCCQ |
| SEQ. ID NO: 1143 | CQPCYCPACCV |
| SEQ. ID NO: 1144 | CQPICCGSSCC |
| SEQ. ID NO: 1145 | CQPRCCETSCC |
| SEQ. ID NO: 1146 | CQPSCCETSCC |
| SEQ. ID NO: 1147 | CQPSCCRPACC |
| SEQ. ID NO: 1148 | CQPSCCVPSCC |
| SEQ. ID NO: 1149 | CQPSCCVSSCC |
| SEQ. ID NO: 1150 | CQPTCCCPSYC |
| SEQ. ID NO: 1151 | CQPTCCGSSCC |
| SEQ. ID NO: 1152 | CQPTCCHPSCC |
| SEQ. ID NO: 1153 | CQPTCCQPTCC |
| SEQ. ID NO: 1154 | CQPTCCRPSCC |
| SEQ. ID NO: 1155 | CQPTCCRPTCC |
| SEQ. ID NO: 1156 | CQPTCCRTTCC |
| SEQ. ID NO: 1157 | CQQACCMPVCC |
| SEQ. ID NO: 1158 | CQQACCVPICC |
| SEQ. ID NO: 1159 | CQQACCVPVCC |
| SEQ. ID NO: 1160 | CQQSCCVPVCC |
| SEQ. ID NO: 1161 | CQQSCCVSVCC |
| SEQ. ID NO: 1162 | CQSNCCVPVCC |
| SEQ. ID NO: 1163 | CQSSCCCPASC |
| SEQ. ID NO: 1164 | CQSSCCKPCCS |
| SEQ. ID NO: 1165 | CQSSCCKPCSC |
| SEQ. ID NO: 1166 | CQSSCCKPYCC |
| SEQ. ID NO: 1167 | CQSSCCNPCCS |
| SEQ. ID NO: 1168 | CQSSCCQSSCC |
| SEQ. ID NO: 1169 | CQSSCCVPVCC |
| SEQ. ID NO: 1170 | CQSSCFKPCCC |
| SEQ. ID NO: 1171 | CQSSCSKPCCC |
| SEQ. ID NO: 1172 | CQSSCYKPCCC |
| SEQ. ID NO: 1173 | CQSVCCQPTCC |
| SEQ. ID NO: 1174 | CQTTCCCPSCV |
| SEQ. ID NO: 1175 | CQTTCCRPSCC |
| SEQ. ID NO: 1176 | CQTTCCRTTCC |
| SEQ. ID NO: 1177 | CRPACCETTCC |
| SEQ. ID NO: 1178 | CRPACCQNTCC |
| SEQ. ID NO: 1179 | CRPCCCLRPVC |
| SEQ. ID NO: 1180 | CRPCCCVRPVC |
| SEQ. ID NO: 1181 | CRPCCWATTCC |
| SEQ. ID NO: 1182 | CRPLCCQTTCC |
| SEQ. ID NO: 1183 | CRPQCCQSVCC |
| SEQ. ID NO: 1184 | CRPQCCQTTCC |
| SEQ. ID NO: 1185 | CRPRCCISSCC |
| SEQ. ID NO: 1186 | CRPSCCESSCC |
| SEQ. ID NO: 1187 | CRPSCCISSCC |
| SEQ. ID NO: 1188 | CRPSCCKPQCC |
| SEQ. ID NO: 1189 | CRPSCCPSCCQ |
| SEQ. ID NO: 1190 | CRPSCCQTTCC |
| SEQ. ID NO: 1191 | CRPSCCRPQCC |
| SEQ. ID NO: 1192 | CRPSCCVSRCC |
| SEQ. ID NO: 1193 | CRPSCCVSSCC |
| SEQ. ID NO: 1194 | CRPTCCQNTCC |
| SEQ. ID NO: 1195 | CRPVCCCEPTC |
| SEQ. ID NO: 1196 | CRPVCCCYSCE |
| SEQ. ID NO: 1197 | CRTTCCHPSCC |
| SEQ. ID NO: 1198 | CRTTCCRPSCC |
| SEQ. ID NO: 1199 | CSCCKPYCSQC |
| SEQ. ID NO: 1200 | CSKPCCCQSSC |
| SEQ. ID NO: 1201 | CSPCCQPTCCR |
| SEQ. ID NO: 1202 | CSPCCVSSCCQ |
| SEQ. ID NO: 1203 | CSQCSCCKPCY |
| SEQ. ID NO: 1204 | CSQCSCYKPCC |
| SEQ. ID NO: 1205 | CSQSNCCKPCC |
| SEQ. ID NO: 1206 | CSQSSCCKPCC |
| SEQ. ID NO: 1207 | CSSSCCQPSCC |
| SEQ. ID NO: 1208 | CTPSCCQPACC |
| SEQ. ID NO: 1209 | CVASCCQPSCC |
| SEQ. ID NO: 1210 | CVPICCCKPVC |
| SEQ. ID NO: 1211 | CVPSCCQPCCH |
| SEQ. ID NO: 1212 | CVPVCCCKPMC |
| SEQ. ID NO: 1213 | CVPVCCCKPVC |
| SEQ. ID NO: 1214 | CVPVCCKPVCC |
| SEQ. ID NO: 1215 | CVSSCCKPQCC |
| SEQ. ID NO: 1216 | CVSSCCQHSCC |
| SEQ. ID NO: 1217 | CVSSCCQPCCH |
| SEQ. ID NO: 1218 | CVSSCCQPCCR |
| SEQ. ID NO: 1219 | CVSSCCQPFCC |
| SEQ. ID NO: 1220 | CVSSCCQPSCC |
| SEQ. ID NO: 1221 | CVSSCCRPQCC |
| SEQ. ID NO: 1222 | CVTRCCSTPCC |
| SEQ. ID NO: 1223 | CVTSCCQPACC |
| SEQ. ID NO: 1224 | CVTSCCQPSCC |
| SEQ. ID NO: 1225 | CVYSCCQPFCC |
| SEQ. ID NO: 1226 | CVYSCCQPSCC |
| SEQ. ID NO: 1227 | CYCPACCVSSC |
| SEQ. ID NO: 1228 | CYKPCCCQSSC |
| SEQ. ID NO: 1229 | CYKPCCCSSGC |
| SEQ. ID NO: 1230 | MCCCVPACSCS |
| SEQ. ID NO: 1231 | NCCVPVCCQCK |
| SEQ. ID NO: 1232 | QCSCCKPCYCS |
| SEQ. ID NO: 1233 | QCSCYKPCCCS |
| SEQ. ID NO: 1234 | SCCVPICCQCK |
| SEQ. ID NO: 1235 | SCCVPVCCQCK |
| SEQ. ID NO: 1236 | SCGCSQCNCCK |
| SEQ. ID NO: 1237 | SCGCSQCSCCK |
| SEQ. ID NO: 1238 | VCCCVPACSCS |
| SEQ. ID NO: 1239 | VCCCVPACSCT |

The present invention is of course in any way restricted to the embodiments herein described and one with ordinary skill in the area can provide many possibilities to modifications and substitutions of technical characteristics by equivalent ones, depending on each situation, as defined in the claims.

The preferred embodiments described above may obviously be combined. The following claims define further preferred embodiments.

What is claimed:

1. A hair composition comprising:
a keratin peptide fragment having about 15% to about 30% cysteine amino acid content, wherein the keratin peptide fragment comprises a peptide sequence at least 90% identical to SEQ ID NO.: 412 or SEQ ID NO.: 409;
wherein the hair composition is a lotion or cream.

2. The hair composition of claim 1, wherein the peptide sequence is at least 95% identical with the to SEQ ID NO.: 412.

3. The hair composition of claim 1, wherein the peptide sequence is at least 95% identical with the to SEQ ID NO.: 409.

4. The hair composition of claim 1, wherein the peptide sequence is identical to SEQ ID NO.: 412.

5. The hair composition of claim 1, wherein the peptide sequence is identical to SEQ ID NO.: 409.

6. A hair composition comprising:
a keratin peptide fragment having about 15% to about 30% cysteine amino acid content, wherein the keratin peptide fragment comprises a peptide sequence at least 90% identical to SEQ ID NO.: 412 or SEQ ID NO.: 409; and
at least one excipient comprising propylene glycol, polysorbate 20, hydrolyzed wheat protein, hydrolyzed wheat starch, potassium sorbate, or an alcohol, or a combination of two or more thereof.

7. The hair composition of claim 6 , wherein the at least one excipient further comprises ether dicaprylic, cetyl ester, behentrimonium chloride, or tocopherol, or a combination of two or more thereof.

8. The hair composition of claim 6, wherein the at least one excipient further comprises potassium hydroxide, acrylate, or phenoxyethanol, or a combination or two or more thereof.

9. The hair composition of claim 1, wherein the keratin peptide fragment forms one or more molecular interactions with hair.

10. The hair composition of claim 9, wherein the one or more molecular interactions is one or more disulfide bonds.

11. A hair composition comprising:
a keratin peptide fragment having about 15% to about 30% cysteine amino acid content, wherein the keratin peptide fragment comprises a peptide sequence at least 90% identical to SEQ ID NO.: 412 or SEQ ID NO.: 409; and
at least one excipient comprising an oil and/or an alcohol.

12. The hair composition of claim 11, comprising the oil and the alcohol.

13. (canceled)

14. (canceled)

15. The hair composition of claim 1, wherein the total number of cysteine amino acids in the keratin peptide fragment is 2 cysteine amino acids.

16. The hair composition of claim 6, wherein the total number of cysteine amino acids in the keratin peptide fragment is 2 cysteine amino acids.

17. The hair composition of claim 11, wherein the total number of cysteine amino acids in the keratin peptide fragment is 2 cysteine amino acids.

18. The hair composition of claim 6, wherein the peptide sequence is identical to SEQ ID NO.: 412.

19. The hair composition of claim 6, wherein the peptide sequence is identical to SEQ ID NO.: 409.

20. The hair composition of claim 11, wherein the peptide sequence is identical to SEQ ID NO.: 412.

21. The hair composition of claim 11, wherein the peptide sequence is identical to SEQ ID NO.: 409.


SEQUENCE LISTING (1239 sequences)

SEQ ID NO: 1 - protein, 10 aa, Homo sapiens
APCAPRPSCG

SEQ ID NO: 2 - protein, 10 aa, Homo sapiens
EACVPSVPCP

SEQ ID NO: 3 - protein, 10 aa, Homo sapiens
ESCGTASGCA

SEQ ID NO: 4 - protein, 10 aa, Homo sapiens
GLCAGTSACL

SEQ ID NO: 5 - protein, 10 aa, Homo sapiens
GVCGPSPPCI

SEQ ID NO: 6 - protein, 10 aa, Homo sapiens
HGCTLPGACN

SEQ ID NO: 7 - protein, 10 aa, Homo sapiens
HSCTLPGACN

SEQ ID NO: 8 - protein, 10 aa, Homo sapiens
KDCLQNSLCE

SEQ ID NO: 9 - protein, 10 aa, Homo sapiens
LPCLPAASCG

SEQ ID NO: 10 - protein, 10 aa, Homo sapiens
LPCYFTGSCN

SEQ ID NO: 11 - protein, 10 aa, Homo sapiens
NFCLPSLSCR

SEQ ID NO: 12 - protein, 10 aa, Homo sapiens
NPCATTNACD

SEQ ID NO: 13 - protein, 10 aa, Homo sapiens
NPCATTNACE

SEQ ID NO: 14 - protein, 10 aa, Homo sapiens
NPCATTNACS

SEQ ID NO: 15 - protein, 10 aa, Homo sapiens
NPCGLRARCG

SEQ ID NO: 16 - protein, 10 aa, Homo sapiens
NPCGPRSRCG

SEQ ID NO: 17 - protein, 10 aa, Homo sapiens
NPCSTPASCT

SEQ ID NO: 18 - protein, 10 aa, Homo sapiens
NPCSTSPSCV

SEQ ID NO: 19 - protein, 10 aa, Homo sapiens
PACTSSSPCS

SEQ ID NO: 20 - protein, 10 aa, Homo sapiens
SKCHESTVCP

SEQ ID NO: 21 - protein, 10 aa, Homo sapiens
SPCVPRTVCV

SEQ ID NO: 22 - protein, 10 aa, Homo sapiens
SSCSVETACL

SEQ ID NO: 23 - protein, 10 aa, Homo sapiens
SVCSSGVNCR

SEQ ID NO: 24 - protein, 10 aa, Homo sapiens
TACPLPGTCH

SEQ ID NO: 25 - protein, 10 aa, Homo sapiens
TNCSPRPICV

SEQ ID NO: 26 - protein, 10 aa, Homo sapiens
TSCVPPAPCT

SEQ ID NO: 27 - protein, 10 aa, Homo sapiens
TTCTSSNTCE

SEQ ID NO: 28 - protein, 10 aa, Homo sapiens
VPCVPSVPCT

SEQ ID NO: 29 - protein, 10 aa, Homo sapiens
ATCGPSACIT

SEQ ID NO: 30 - protein, 10 aa, Homo sapiens
GPCISNPCGL

SEQ ID NO: 31 - protein, 10 aa, Homo sapiens
GPCLSNPCTS

SEQ ID NO: 32 - protein, 10 aa, Homo sapiens
GSCVTNPCGP

SEQ ID NO: 33 - protein, 10 aa, Homo sapiens
LTCFSITCSS

SEQ ID NO: 34 - protein, 10 aa, Homo sapiens
NPCSTPSCTT

SEQ ID NO: 35 - protein, 10 aa, Homo sapiens
PSCVTAPCAP

SEQ ID NO: 36 - protein, 10 aa, Homo sapiens
SDCSSTHCSP

SEQ ID NO: 37 - protein, 10 aa, Homo sapiens
SLCLPPTCHT

SEQ ID NO: 38 - protein, 10 aa, Homo sapiens
SLCNLGSCGP

SEQ ID NO: 39 - protein, 10 aa, Homo sapiens
SPCLVGNCAW

SEQ ID NO: 40 - protein, 10 aa, Homo sapiens
TACLPGTCAT

SEQ ID NO: 41 - protein, 10 aa, Homo sapiens
TSCLPALCLP

SEQ ID NO: 42 - protein, 10 aa, Homo sapiens
TSCSSRPCVP

SEQ ID NO: 43 - protein, 10 aa, Homo sapiens
TTCGGGSCGV

SEQ ID NO: 44 - protein, 10 aa, Homo sapiens
VNCRPELCLG

SEQ ID NO: 45 - protein, 10 aa, Homo sapiens
YVCQPMACLP

SEQ ID NO: 46 - protein, 10 aa, Homo sapiens
AFSCISACGP

SEQ ID NO: 47 - protein, 10 aa, Homo sapiens
GSVCSAPCNG

SEQ ID NO: 48 - protein, 10 aa, Homo sapiens
GVVCGDLCAS

SEQ ID NO: 49 - protein, 10 aa, Homo sapiens
GVVCGDLCVS

SEQ ID NO: 50 - protein, 10 aa, Homo sapiens
LTGCLLPCYF

SEQ ID NO: 51 - protein, 10 aa, Homo sapiens
NEDCKLPCNP

SEQ ID NO: 52 - protein, 10 aa, Homo sapiens
NFSCVSACGP

SEQ ID NO: 53 - protein, 10 aa, Homo sapiens
PPTCHTACPL

SEQ ID NO: 54 - protein, 10 aa, Homo sapiens
PQPCATACKP

SEQ ID NO: 55 - protein, 10 aa, Homo sapiens
SEDCKLPCNP

SEQ ID NO: 56 - protein, 10 aa, Homo sapiens
SLGCRTSCSS

SEQ ID NO: 57 - protein, 10 aa, Homo sapiens
SLSCRTSCSS

SEQ ID NO: 58 - protein, 10 aa, Homo sapiens
SSSCPLGCTM

SEQ ID NO: 59 - protein, 10 aa, Homo sapiens
TGSCNSPCLV

SEQ ID NO: 60 - protein, 10 aa, Homo sapiens
TSSCPLGCTM

SEQ ID NO: 61 - protein, 10 aa, Homo sapiens
VGSCGSSCRK

SEQ ID NO: 62 - protein, 10 aa, Homo sapiens
VGVCGGSCKR

SEQ ID NO: 63 - protein, 10 aa, Homo sapiens
VSNCNWFCEG

SEQ ID NO: 64 - protein, 10 aa, Homo sapiens
ACGPRPGRCC

SEQ ID NO: 65 - protein, 10 aa, Homo sapiens
ACGPRPSRCC

SEQ ID NO: 66 - protein, 10 aa, Homo sapiens
CAPRPSCGPC

SEQ ID NO: 67 - protein, 10 aa, Homo sapiens
CEPCSAYVIC

SEQ ID NO: 68 - protein, 10 aa, Homo sapiens
CGLRARCGPC

SEQ ID NO: 69 - protein, 10 aa, Homo sapiens
CGPRPGRCCI

SEQ ID NO: 70 - protein, 10 aa, Homo sapiens
CGPRPSRCCI

SEQ ID NO: 71 - protein, 10 aa, Homo sapiens
CGPRSRCGPC

SEQ ID NO: 72 - protein, 10 aa, Homo sapiens
CGTSQKGCCN

SEQ ID NO: 73 - protein, 10 aa, Homo sapiens
CHGCTLPGAC

SEQ ID NO: 74 - protein, 10 aa, Homo sapiens
CHSCTLPGAC

SEQ ID NO: 75 - protein, 10 aa, Homo sapiens
CLPCLPAASC

SEQ ID NO: 76 - protein, 10 aa, Homo sapiens
CLPPTCHTAC

SEQ ID NO: 77 - protein, 10 aa, Homo sapiens
CLSNPCTSCV

SEQ ID NO: 78 - protein, 10 aa, Homo sapiens
CLVGNCAWCE

SEQ ID NO: 79 - protein, 10 aa, Homo sapiens
CNPCSTPASC

SEQ ID NO: 80 - protein, 10 aa, Homo sapiens
CNPCSTPSCT

SEQ ID NO: 81 - protein, 10 aa, Homo sapiens
CNPCSTSPSC

SEQ ID NO: 82 - protein, 10 aa, Homo sapiens
CNSPCLVGNC

SEQ ID NO: 83 - protein, 10 aa, Homo sapiens
CRTSCSSRPC

SEQ ID NO: 84 - protein, 10 aa, Homo sapiens
CSLKEHCSAC

SEQ ID NO: 85 - protein, 10 aa, Homo sapiens
CSPRPICVPC

SEQ ID NO: 86 - protein, 10 aa, Homo sapiens
CSSTMSYSCC

SEQ ID NO: 87 - protein, 10 aa, Homo sapiens
CSTPASCTSC

SEQ ID NO: 88 - protein, 10 aa, Homo sapiens
CSTPSCTTCV

SEQ ID NO: 89 - protein, 10 aa, Homo sapiens
CTSCVPPAPC

SEQ ID NO: 90 - protein, 10 aa, Homo sapiens
CTSSNTCEPC

SEQ ID NO: 91 - protein, 10 aa, Homo sapiens
CVPPAPCTPC

SEQ ID NO: 92 - protein, 10 aa, Homo sapiens
CVPPSCHGCT

SEQ ID NO: 93 - protein, 10 aa, Homo sapiens
CVPPSCHSCT

SEQ ID NO: 94 - protein, 10 aa, Homo sapiens
DCKLPCNPCA

SEQ ID NO: 95 - protein, 10 aa, Homo sapiens
DCKLPCNPCS

SEQ ID NO: 96 - protein, 10 aa, Homo sapiens
PCGTSQKGCC

SEQ ID NO: 97 - protein, 10 aa, Homo sapiens
PCLSNPCTSC

SEQ ID NO: 98 - protein, 10 aa, Homo sapiens
PCLVGNCAWC

SEQ ID NO: 99 - protein, 10 aa, Homo sapiens
PCNPCSTPSC

SEQ ID NO: 100 - protein, 10 aa, Homo sapiens
PCSTPSCTTC

SEQ ID NO: 101 - protein, 10 aa, Homo sapiens
PCTTCGPTCG

SEQ ID NO: 102 - protein, 10 aa, Homo sapiens
PCVPPSCHGC

SEQ ID NO: 103 - protein, 10 aa, Homo sapiens
PCVPPSCHSC

SEQ ID NO: 104 - protein, 10 aa, Homo sapiens
SCCLPSLGCR

SEQ ID NO: 105 - protein, 10 aa, Homo sapiens
SCSEELQCCQ

SEQ ID NO: 106 - protein, 10 aa, Homo sapiens
SCSPCSTTCT

SEQ ID NO: 107 - protein, 10 aa, Homo sapiens
ASCSTSGTCG

SEQ ID NO: 108 - protein, 10 aa, Homo sapiens
ASCYIPVGCQ

SEQ ID NO: 109 - protein, 10 aa, Homo sapiens
ASCYVPVSCQ

SEQ ID NO: 110 - protein, 10 aa, Homo sapiens
AVCTLPSSCQ

SEQ ID NO: 111 - protein, 10 aa, Homo sapiens
DLCPTSVSCG

SEQ ID NO: 112 - protein, 10 aa, Homo sapiens
EICWEPTSCQ

SEQ ID NO: 113 - protein, 10 aa, Homo sapiens
ETCGEPTSCQ

SEQ ID NO: 114 - protein, 10 aa, Homo sapiens
ETCNETTSCQ

SEQ ID NO: 115 - protein, 10 aa, Homo sapiens
ETCWRPNSCQ

SEQ ID NO: 116 - protein, 10 aa, Homo sapiens
GYCGYRPFCF

SEQ ID NO: 117 - protein, 10 aa, Homo sapiens
KTCWEPASCQ

SEQ ID NO: 118 - protein, 10 aa, Homo sapiens
KTCWEPTSCQ

SEQ ID NO: 119 - protein, 10 aa, Homo sapiens
LDCVDTTPCK

SEQ ID NO: 120 - protein, 10 aa, Homo sapiens
LGCGYGSFCG

SEQ ID NO: 121 - protein, 10 aa, Homo sapiens
NSCGYGSGCG

SEQ ID NO: 122 - protein, 10 aa, Homo sapiens
NYCPSNTMCE

SEQ ID NO: 123 - protein, 10 aa, Homo sapiens
PACVTSYSCR

SEQ ID NO: 124 - protein, 10 aa, Homo sapiens
PDCHVEGTCL

SEQ ID NO: 125 - protein, 10 aa, Homo sapiens
PDCRVEGTCL

SEQ ID NO: 126 - protein, 10 aa, Homo sapiens
PICSEPSPCS

SEQ ID NO: 127 - protein, 10 aa, Homo sapiens
PICYIFKPCQ

SEQ ID NO: 128 - protein, 10 aa, Homo sapiens
PLCYISNSCQ

SEQ ID NO: 129 - protein, 10 aa, Homo sapiens
PPCGQPTPCS

SEQ ID NO: 130 - protein, 10 aa, Homo sapiens
PPCHIPQPCV

SEQ ID NO: 131 - protein, 10 aa, Homo sapiens
PSCGRLASCG

SEQ ID NO: 132 - protein, 10 aa, Homo sapiens
PSCSESSICQ

SEQ ID NO: 133 - protein, 10 aa, Homo sapiens
PSCSEVTSCP

SEQ ID NO: 134 - protein, 10 aa, Homo sapiens
PSCSTSGTCG

SEQ ID NO: 135 - protein, 10 aa, Homo sapiens
PSCSVSSGCQ

SEQ ID NO: 136 - protein, 10 aa, Homo sapiens
PSCTESDSCK

SEQ ID NO: 137 - protein, 10 aa, Homo sapiens
PSCYQTSSCG

SEQ ID NO: 138 - protein, 10 aa, Homo sapiens
PTCFLLNSCQ

SEQ ID NO: 139 - protein, 10 aa, Homo sapiens
PTCSVTSSCQ

SEQ ID NO: 140 - protein, 10 aa, Homo sapiens
PTCWLLNNCH

SEQ ID NO: 141 - protein, 10 aa, Homo sapiens
PTCYQRTSCV

SEQ ID NO: 142 - protein, 10 aa, Homo sapiens
PTCYRRTSCV

SEQ ID NO: 143 - protein, 10 aa, Homo sapiens
PTCYVVKRCP

SEQ ID NO: 144 - protein, 10 aa, Homo sapiens
PVCFEATICE

SEQ ID NO: 145 - protein, 10 aa, Homo sapiens
PVCFEATVCE

SEQ ID NO: 146 - protein, 10 aa, Homo sapiens
PVCSRPASCS

SEQ ID NO: 147 - protein, 10 aa, Homo sapiens
PVCSWVPACS

SEQ ID NO: 148 - protein, 10 aa, Homo sapiens
QTCNESSYCL

SEQ ID NO: 149 - protein, 10 aa, Homo sapiens
QTCWEPTSCQ

SEQ ID NO: 150 - protein, 10 aa, Homo sapiens
SFCRLGYGCG

SEQ ID NO: 151 - protein, 10 aa, Homo sapiens
SFCRRGSGCG

SEQ ID NO: 152 - protein, 10 aa, Homo sapiens
SLCGYGYGCG

SEQ ID NO: 153 - protein, 10 aa, Homo sapiens
SLCSTEVSCG

SEQ ID NO: 154 - protein, 10 aa, Homo sapiens
SNCFGQLNCL

SEQ ID NO: 155 - protein, 10 aa, Homo sapiens
SPCGQPTPCS

SEQ ID NO: 156 - protein, 10 aa, Homo sapiens
SSCDQSSSCA

SEQ ID NO: 157 - protein, 10 aa, Homo sapiens
SSCGQSSSCA

SEQ ID NO: 158 - protein, 10 aa, Homo sapiens
SVCPEPVSCP

SEQ ID NO: 159 - protein, 10 aa, Homo sapiens
TFCSFDKSCR

SEQ ID NO: 160 - protein, 10 aa, Homo sapiens
TICSSDKSCR

SEQ ID NO: 161 - protein, 10 aa, Homo sapiens
TLCVESSPCH

SEQ ID NO: 162 - protein, 10 aa, Homo sapiens
TPCYQQSSCQ

SEQ ID NO: 163 - protein, 10 aa, Homo sapiens
VTCSRQTTCI

SEQ ID NO: 164 - protein, 10 aa, Homo sapiens
YGCGYGSGCG

SEQ ID NO: 165 - protein, 10 aa, Homo sapiens
YGCGYGSGCR

SEQ ID NO: 166 - protein, 10 aa, Homo sapiens
YGCIHSTHCG

SEQ ID NO: 167 - protein, 10 aa, Homo sapiens
AACEPSACQS

SEQ ID NO: 168 - protein, 10 aa, Homo sapiens
AACEPSPCQS

SEQ ID NO: 169 - protein, 10 aa, Homo sapiens
AACTMSVCSS

SEQ ID NO: 170 - protein, 10 aa, Homo sapiens
ADCLGGICLP

SEQ ID NO: 171 - protein, 10 aa, Homo sapiens
ALCLPSSCHS

SEQ ID NO: 172 - protein, 10 aa, Homo sapiens
ALCSPSTCQL

SEQ ID NO: 173 - protein, 10 aa, Homo sapiens
APCLALVCAP

SEQ ID NO: 174 - protein, 10 aa, Homo sapiens
APCLSLVCTP

SEQ ID NO: 175 - protein, 10 aa, Homo sapiens
APCLTLVCTP

SEQ ID NO: 176 - protein, 10 aa, Homo sapiens
APCVALLCRP

SEQ ID NO: 177 - protein, 10 aa, Homo sapiens
ASCGSLLCRP

SEQ ID NO: 178 - protein, 10 aa, Homo sapiens
ASCLSFLCRP

SEQ ID NO: 179 - protein, 10 aa, Homo sapiens
ASCVSLLCRP

SEQ ID NO: 180 - protein, 10 aa, Homo sapiens
AVCEPSPCQS

SEQ ID NO: 181 - protein, 10 aa, Homo sapiens
AVCLPVSCQS

SEQ ID NO: 182 - protein, 10 aa, Homo sapiens
AVCVPVRCQS

SEQ ID NO: 183 - protein, 10 aa, Homo sapiens
AVCVPVSCQS

SEQ ID NO: 184 - protein, 10 aa, Homo sapiens
DLCSPSTCQL

SEQ ID NO: 185 - protein, 10 aa, Homo sapiens
DSCGSSSCGP

SEQ ID NO: 186 - protein, 10 aa, Homo sapiens
DSCVQSNCFP

SEQ ID NO: 187 - protein, 10 aa, Homo sapiens
FNCSTRNCSS

SEQ ID NO: 188 - protein, 10 aa, Homo sapiens
GGCGSYGCSQ

SEQ ID NO: 189 - protein, 10 aa, Homo sapiens
GSCGFGSCYG

SEQ ID NO: 190 - protein, 10 aa, Homo sapiens
GSCSSRKCFS

SEQ ID NO: 191 - protein, 10 aa, Homo sapiens
GVCLPSTCPH

SEQ ID NO: 192 - protein, 10 aa, Homo sapiens
HSCEGYLCYS

SEQ ID NO: 193 - protein, 10 aa, Homo sapiens
IVCAAPSCQS

SEQ ID NO: 194 - protein, 10 aa, Homo sapiens
KTCSTTGCDP

SEQ ID NO: 195 - protein, 10 aa, Homo sapiens
LACVSQPCQS

SEQ ID NO: 196 - protein, 10 aa, Homo sapiens
LGCGYGGCGY

SEQ ID NO: 197 - protein, 10 aa, Homo sapiens
LSCGSRSCSS

SEQ ID NO: 198 - protein, 10 aa, Homo sapiens
LVCTPVSCVS

SEQ ID NO: 199 - protein, 10 aa, Homo sapiens
NGCQETYCEP

SEQ ID NO: 200 - protein, 10 aa, Homo sapiens
NSCRSLSCGS

SEQ ID NO: 201 - protein, 10 aa, Homo sapiens
PACVISTCPR

SEQ ID NO: 202 - protein, 10 aa, Homo sapiens
PGCLNQSCGS

SEQ ID NO: 203 - protein, 10 aa, Homo sapiens
PPCGTAPCLT

SEQ ID NO: 204 - protein, 10 aa, Homo sapiens
PPCTTALCRP

SEQ ID NO: 205 - protein, 10 aa, Homo sapiens
PPCYLVSCTP

SEQ ID NO: 206 - protein, 10 aa, Homo sapiens
PRCTRPICEP

SEQ ID NO: 207 - protein, 10 aa, Homo sapiens
PSCPVSSCAQ

SEQ ID NO: 208 - protein, 10 aa, Homo sapiens
PSCQPSVCVP

SEQ ID NO: 209 - protein, 10 aa, Homo sapiens
PSCSVSNCYQ

SEQ ID NO: 210 - protein, 10 aa, Homo sapiens
PSCSVSSCAQ

SEQ ID NO: 211 - protein, 10 aa, Homo sapiens
PSCTSVLCRP

SEQ ID NO: 212 - protein, 10 aa, Homo sapiens
PTCKSPSCEP

SEQ ID NO: 213 - protein, 10 aa, Homo sapiens
PTCVISSCPR

SEQ ID NO: 214 - protein, 10 aa, Homo sapiens
PTCVISTCPR

SEQ ID NO: 215 - protein, 10 aa, Homo sapiens
PTCYQTICFR

SEQ ID NO: 216 - protein, 10 aa, Homo sapiens
PVCGGVSCHT

SEQ ID NO: 217 - protein, 10 aa, Homo sapiens
PVCGRVSCHT

SEQ ID NO: 218 - protein, 10 aa, Homo sapiens
PVCNKPVCFV

SEQ ID NO: 219 - protein, 10 aa, Homo sapiens
PVCPTPTCSV

SEQ ID NO: 220 - protein, 10 aa, Homo sapiens
PVCRSTYCVP

SEQ ID NO: 221 - protein, 10 aa, Homo sapiens
PVCSKSVCYV

SEQ ID NO: 222 - protein, 10 aa, Homo sapiens
PVCSRPACYS

SEQ ID NO: 223 - protein, 10 aa, Homo sapiens
PVCYVPTCSE

SEQ ID NO: 224 - protein, 10 aa, Homo sapiens
QFCLSKSCQP

SEQ ID NO: 225 - protein, 10 aa, Homo sapiens
RPCERTACQS

SEQ ID NO: 226 - protein, 10 aa, Homo sapiens
RSCQTSFCGF

SEQ ID NO: 227 - protein, 10 aa, Homo sapiens
RSCSSLGCGS

SEQ ID NO: 228 - protein, 10 aa, Homo sapiens
RSCYSVGCGS

SEQ ID NO: 229 - protein, 10 aa, Homo sapiens
RVCLPGSCDS

SEQ ID NO: 230 - protein, 10 aa, Homo sapiens
SFCGFPSCST

SEQ ID NO: 231 - protein, 10 aa, Homo sapiens
SFCGYPSCST

SEQ ID NO: 232 - protein, 10 aa, Homo sapiens
SGCDPASCQP

SEQ ID NO: 233 - protein, 10 aa, Homo sapiens
SGCGGSGCGG

SEQ ID NO: 234 - protein, 10 aa, Homo sapiens
SGCQPSSCLA

SEQ ID NO: 235 - protein, 10 aa, Homo sapiens
SHCQPPHCQL

SEQ ID NO: 236 - protein, 10 aa, Homo sapiens
SICQPATCVA

SEQ ID NO: 237 - protein, 10 aa, Homo sapiens
SLCVPVSCRP

SEQ ID NO: 238 - protein, 10 aa, Homo sapiens
SNCLPTSCQP

SEQ ID NO: 239 - protein, 10 aa, Homo sapiens
SPCLVSSCQP

SEQ ID NO: 240 - protein, 10 aa, Homo sapiens
SPCQQSSCQE

SEQ ID NO: 241 - protein, 10 aa, Homo sapiens
SPCQQSYCVP

SEQ ID NO: 242 - protein, 10 aa, Homo sapiens
SPCSPAVCVS

SEQ ID NO: 243 - protein, 10 aa, Homo sapiens
SRCQQPSCQP

SEQ ID NO: 244 - protein, 10 aa, Homo sapiens
SRCYRPHCGQ

SEQ ID NO: 245 - protein, 10 aa, Homo sapiens
SSCAPIYCRR

SEQ ID NO: 246 - protein, 10 aa, Homo sapiens
SSCAPVYCRR

SEQ ID NO: 247 - protein, 10 aa, Homo sapiens
SSCGKGGCGS

SEQ ID NO: 248 - protein, 10 aa, Homo sapiens
SSCGKRGCGS

SEQ ID NO: 249 - protein, 10 aa, Homo sapiens
SSCLPVSCRP

SEQ ID NO: 250 - protein, 10 aa, Homo sapiens
SSCQPAYCTS

SEQ ID NO: 251 - protein, 10 aa, Homo sapiens
SSCQPSYCRQ

SEQ ID NO: 252 - protein, 10 aa, Homo sapiens
SSCQPVVCEP

SEQ ID NO: 253 - protein, 10 aa, Homo sapiens
SSCTAVVCRP

SEQ ID NO: 254 - protein, 10 aa, Homo sapiens
SSCYQPFCRS

SEQ ID NO: 255 - protein, 10 aa, Homo sapiens
SSCYRPICGS

SEQ ID NO: 256 - protein, 10 aa, Homo sapiens
SSCYRPTCGS

SEQ ID NO: 257 - protein, 10 aa, Homo sapiens
SVCMSGSCQA

SEQ ID NO: 258 - protein, 10 aa, Homo sapiens
SVCSDQGCDQ

SEQ ID NO: 259 - protein, 10 aa, Homo sapiens
SVCSDQGCGL

SEQ ID NO: 260 - protein, 10 aa, Homo sapiens
SVCSDQGCGQ

SEQ ID NO: 261 - protein, 10 aa, Homo sapiens
SVCSDQGCSQ

SEQ ID NO: 262 - protein, 10 aa, Homo sapiens
SVCSDQSCGQ

SEQ ID NO: 263 - protein, 10 aa, Homo sapiens
SVCSHQGCGQ

SEQ ID NO: 264 - protein, 10 aa, Homo sapiens
SVCSHQGCGR

SEQ ID NO: 265 - protein, 10 aa, Homo sapiens
SVCVPVSCRP

SEQ ID NO: 266 - protein, 10 aa, Homo sapiens
SYCRQASCVS

SEQ ID NO: 267 - protein, 10 aa, Homo sapiens
TACEPSACQS

SEQ ID NO: 268 - protein, 10 aa, Homo sapiens
TICTASPCQP

SEQ ID NO: 269 - protein, 10 aa, Homo sapiens
TSCPETSCLP

SEQ ID NO: 270 - protein, 10 aa, Homo sapiens
TSCQMTNCEQ

SEQ ID NO: 271 - protein, 10 aa, Homo sapiens
TSCQPVHCET

SEQ ID NO: 272 - protein, 10 aa, Homo sapiens
TSCQPVLCKS

SEQ ID NO: 273 - protein, 10 aa, Homo sapiens
TSCQPVLCVP

SEQ ID NO: 274 - protein, 10 aa, Homo sapiens
TSCVGFVCQP

SEQ ID NO: 275 - protein, 10 aa, Homo sapiens
TSCVSNPCQV

SEQ ID NO: 276 - protein, 10 aa, Homo sapiens
TTCFQPTCVS

SEQ ID NO: 277 - protein, 10 aa, Homo sapiens
TTCFQPTCVT

SEQ ID NO: 278 - protein, 10 aa, Homo sapiens
TTCFQPTCVY

SEQ ID NO: 279 - protein, 10 aa, Homo sapiens
TTCISNPCST

SEQ ID NO: 280 - protein, 10 aa, Homo sapiens
TWCQGSSCQP

SEQ ID NO: 281 - protein, 10 aa, Homo sapiens
VGCQSSVCVP

SEQ ID NO: 282 - protein, 10 aa, Homo sapiens
VPCQPSTCVF

SEQ ID NO: 283 - protein, 10 aa, Homo sapiens
VSCEPSPCQS

SEQ ID NO: 284 - protein, 10 aa, Homo sapiens
VSCGGPICLP

SEQ ID NO: 285 - protein, 10 aa, Homo sapiens
VSCKPVLCVA

SEQ ID NO: 286 - protein, 10 aa, Homo sapiens
VSCPSTSCRP

SEQ ID NO: 287 - protein, 10 aa, Homo sapiens
VSCQSSVCMP

SEQ ID NO: 288 - protein, 10 aa, Homo sapiens
VSCTRIVCVA

SEQ ID NO: 289 - protein, 10 aa, Homo sapiens
VTCEPSPCQS

SEQ ID NO: 290 - protein, 10 aa, Homo sapiens
VTCQTTVCRP

SEQ ID NO: 291 - protein, 10 aa, Homo sapiens
YGCGYEGCRY

SEQ ID NO: 292 - protein, 10 aa, Homo sapiens
AGSCQPSCSE

SEQ ID NO: 293 - protein, 10 aa, Homo sapiens
ALLCRPLCGV

SEQ ID NO: 294 - protein, 10 aa, Homo sapiens
ALVCEPVCLR

SEQ ID NO: 295 - protein, 10 aa, Homo sapiens
ATICEPSCSV

SEQ ID NO: 296 - protein, 10 aa, Homo sapiens
ATTCEPSCSV

SEQ ID NO: 297 - protein, 10 aa, Homo sapiens
ATVCEPSCSV

SEQ ID NO: 298 - protein, 10 aa, Homo sapiens
EGTCLPPCYL

SEQ ID NO: 299 - protein, 10 aa, Homo sapiens
FSTCRPSCSG

SEQ ID NO: 300 - protein, 10 aa, Homo sapiens
GFVCQPMCSH

SEQ ID NO: 301 - protein, 10 aa, Homo sapiens
GLDCGYGCGY

SEQ ID NO: 302 - protein, 10 aa, Homo sapiens
GLGCGYGCGY

SEQ ID NO: 303 - protein, 10 aa, Homo sapiens
GLGCSYGCGH

SEQ ID NO: 304 - protein, 10 aa, Homo sapiens
GLGCSYGCGL

SEQ ID NO: 305 - protein, 10 aa, Homo sapiens
GSGCGYGCGY

SEQ ID NO: 306 - protein, 10 aa, Homo sapiens
GTGCGYGCGY

SEQ ID NO: 307 - protein, 10 aa, Homo sapiens
GVSCHTTCYR

SEQ ID NO: 308 - protein, 10 aa, Homo sapiens
GYACNFPCSY

SEQ ID NO: 309 - protein, 10 aa, Homo sapiens
GYGCGYGCGF

SEQ ID NO: 310 - protein, 10 aa, Homo sapiens
HSPCQASCYV

SEQ ID NO: 311 - protein, 10 aa, Homo sapiens
HTSCSPACQP

SEQ ID NO: 312 - protein, 10 aa, Homo sapiens
HTSCSSGCQP

SEQ ID NO: 313 - protein, 10 aa, Homo sapiens
IRWCHPDCHV

SEQ ID NO: 314 - protein, 10 aa, Homo sapiens
IRWCRPDCRV

SEQ ID NO: 315 - protein, 10 aa, Homo sapiens
ISSCGTGCGI

SEQ ID NO: 316 - protein, 10 aa, Homo sapiens
KGGCGSGCGG

SEQ ID NO: 317 - protein, 10 aa, Homo sapiens
KGGCGSSCSQ

SEQ ID NO: 318 - protein, 10 aa, Homo sapiens
LVTCQDSCGS

SEQ ID NO: 319 - protein, 10 aa, Homo sapiens
LVTCQESCQP

SEQ ID NO: 320 - protein, 10 aa, Homo sapiens
MSICSSACTD

SEQ ID NO: 321 - protein, 10 aa, Homo sapiens
MSICSSACTN

SEQ ID NO: 322 - protein, 10 aa, Homo sapiens
MSVCSSACSD

SEQ ID NO: 323 - protein, 10 aa, Homo sapiens
PAICEPSCSV

SEQ ID NO: 324 - protein, 10 aa, Homo sapiens
PASCQKSCYR

SEQ ID NO: 325 - protein, 10 aa, Homo sapiens
PIYCRRTCYH

SEQ ID NO: 326 - protein, 10 aa, Homo sapiens
PNSCQTLCVE

SEQ ID NO: 327 - protein, 10 aa, Homo sapiens
PQPCVPTCFL

SEQ ID NO: 328 - protein, 10 aa, Homo sapiens
PSACQSGCTS

SEQ ID NO: 329 - protein, 10 aa, Homo sapiens
PSPCEPSCSE

SEQ ID NO: 330 - protein, 10 aa, Homo sapiens
PSPCQASCYI

SEQ ID NO: 331 - protein, 10 aa, Homo sapiens
PSPCQSGCIS

SEQ ID NO: 332 - protein, 10 aa, Homo sapiens
PSPCQSGCTD

SEQ ID NO: 333 - protein, 10 aa, Homo sapiens
PSPCQSGCTS

SEQ ID NO: 334 - protein, 10 aa, Homo sapiens
PTACQPTCYQ

SEQ ID NO: 335 - protein, 10 aa, Homo sapiens
PTACQPTCYR

SEQ ID NO: 336 - protein, 10 aa, Homo sapiens
PTPCSTTCRT

SEQ ID NO: 337 - protein, 10 aa, Homo sapiens
PTSCQKSCYR

SEQ ID NO: 338 - protein, 10 aa, Homo sapiens
PTSCQPSCES

SEQ ID NO: 339 - protein, 10 aa, Homo sapiens
PTSCQTSCTL

SEQ ID NO: 340 - protein, 10 aa, Homo sapiens
PVICEPSCSV

SEQ ID NO: 341 - protein, 10 aa, Homo sapiens
PVSCVPVCSG

SEQ ID NO: 342 - protein, 10 aa, Homo sapiens
PVTCVPRCTR

SEQ ID NO: 343 - protein, 10 aa, Homo sapiens
PVYCRRTCYH

SEQ ID NO: 344 - protein, 10 aa, Homo sapiens
PVYCRRTCYY

SEQ ID NO: 345 - protein, 10 aa, Homo sapiens
PVYCVPVCSG

SEQ ID NO: 346 - protein, 10 aa, Homo sapiens
QPGCESPCEP

SEQ ID NO: 347 - protein, 10 aa, Homo sapiens
QQSCVSSCRR

SEQ ID NO: 348 - protein, 10 aa, Homo sapiens
QTSCGSSCGQ

SEQ ID NO: 349 - protein, 10 aa, Homo sapiens
QTTCHPSCGM

SEQ ID NO: 350 - protein, 10 aa, Homo sapiens
QTTCRPSCGV

SEQ ID NO: 351 - protein, 10 aa, Homo sapiens
RGGCGSGCGG

SEQ ID NO: 352 - protein, 10 aa, Homo sapiens
RLACYSLCSG

SEQ ID NO: 353 - protein, 10 aa, Homo sapiens
RPACYRPCYS

SEQ ID NO: 354 - protein, 10 aa, Homo sapiens
RPFCFRRCYS

SEQ ID NO: 355 - protein, 10 aa, Homo sapiens
RPICRPICSG

SEQ ID NO: 356 - protein, 10 aa, Homo sapiens
RPLCYRRCYS

SEQ ID NO: 357 - protein, 10 aa, Homo sapiens
RSPCQASCYV

SEQ ID NO: 358 - protein, 10 aa, Homo sapiens
RVSCHTTCYR

SEQ ID NO: 359 - protein, 10 aa, Homo sapiens
SAICRPTCPR

SEQ ID NO: 360 - protein, 10 aa, Homo sapiens
SDSCKRDCKK

SEQ ID NO: 361 - protein, 10 aa, Homo sapiens
SEGCGSGCGG

SEQ ID NO: 362 - protein, 10 aa, Homo sapiens
SFLCRPACSR

SEQ ID NO: 363 - protein, 10 aa, Homo sapiens
SGGCGSGCGG

SEQ ID NO: 364 - protein, 10 aa, Homo sapiens
SGGCGSSCGG

SEQ ID NO: 365 - protein, 10 aa, Homo sapiens
SGSCQAACGQ

SEQ ID NO: 366 - protein, 10 aa, Homo sapiens
SLLCHPVCKS

SEQ ID NO: 367 - protein, 10 aa, Homo sapiens
SLLCHPVCRS

SEQ ID NO: 368 - protein, 10 aa, Homo sapiens
SLLCRPACSP

SEQ ID NO: 369 - protein, 10 aa, Homo sapiens
SLLCRPACSR

SEQ ID NO: 370 - protein, 10 aa, Homo sapiens
SLLCRPICRP

SEQ ID NO: 371 - protein, 10 aa, Homo sapiens
SLLCRPMCSR

SEQ ID NO: 372 - protein, 10 aa, Homo sapiens
SLLCRPTCSR

SEQ ID NO: 373 - protein, 10 aa, Homo sapiens
SLLCRPVCQP

SEQ ID NO: 374 - protein, 10 aa, Homo sapiens
SLLCRPVCRP

SEQ ID NO: 375 - protein, 10 aa, Homo sapiens
SLLCRPVCRS

SEQ ID NO: 376 - protein, 10 aa, Homo sapiens
SLLCRPVCSR

SEQ ID NO: 377 - protein, 10 aa, Homo sapiens
SNPCQVTCSR

SEQ ID NO: 378 - protein, 10 aa, Homo sapiens
SRGCGSGCGG

SEQ ID NO: 379 - protein, 10 aa, Homo sapiens
SRSCQSPCYR

SEQ ID NO: 380 - protein, 10 aa, Homo sapiens
SRSCQSSCYR

SEQ ID NO: 381 - protein, 10 aa, Homo sapiens
SSGCGYGCGY

SEQ ID NO: 382 - protein, 10 aa, Homo sapiens
SSGCPMACPG

SEQ ID NO: 383 - protein, 10 aa, Homo sapiens
SSICQPICSE

SEQ ID NO: 384 - protein, 10 aa, Homo sapiens
SSPCHTSCYY

SEQ ID NO: 385 - protein, 10 aa, Homo sapiens
SSPCQPTCYV

SEQ ID NO: 386 - protein, 10 aa, Homo sapiens
SSPCQQSCYV

SEQ ID NO: 387 - protein, 10 aa, Homo sapiens
SSPCQTSCYR

SEQ ID NO: 388 - protein, 10 aa, Homo sapiens
SSSCQQSCRV

SEQ ID NO: 389 - protein, 10 aa, Homo sapiens
STVCQPACGV

SEQ ID NO: 390 - protein, 10 aa, Homo sapiens
TDNCQETCGE

SEQ ID NO: 391 - protein, 10 aa, Homo sapiens
TQPCYEPCLP

SEQ ID NO: 392 - protein, 10 aa, Homo sapiens
TSSCGTGCGI

SEQ ID NO: 393 - protein, 10 aa, Homo sapiens
TSSCQPSCGR

SEQ ID NO: 394 - protein, 10 aa, Homo sapiens
TSSCTTPCYQ

SEQ ID NO: 395 - protein, 10 aa, Homo sapiens
TSVCLPGCLN

SEQ ID NO: 396 - protein, 10 aa, Homo sapiens
TTVCLPGCLN

SEQ ID NO: 397 - protein, 10 aa, Homo sapiens
VANCQAPCST

SEQ ID NO: 398 - protein, 10 aa, Homo sapiens
VDDCPESCWP

SEQ ID NO: 399 - protein, 10 aa, Homo sapiens
VKRCPSVCPE

SEQ ID NO: 400 - protein, 10 aa, Homo sapiens
VSSCQPSCSE

SEQ ID NO: 401 - protein, 10 aa, Homo sapiens
YEGCRYGCGH

SEQ ID NO: 402 - protein, 10 aa, Homo sapiens
YGRCRHGCHS

SEQ ID NO: 403 - protein, 10 aa, Homo sapiens
YGYCRPSCYG

SEQ ID NO: 404 - protein, 10 aa, Homo sapiens
YRDCQKTCWE

SEQ ID NO: 405 - protein, 10 aa, Homo sapiens
YRGCQEICWE

SEQ ID NO: 406 - protein, 10 aa, Homo sapiens
YRGCQETCWR

SEQ ID NO: 407 - protein, 10 aa, Homo sapiens
YRGCQQTCWE

SEQ ID NO: 408 - protein, 10 aa, Homo sapiens
YRSCRPSCYG

SEQ ID NO: 409 - protein, 10 aa, Homo sapiens
GGVCGPSPPC

SEQ ID NO: 410 - protein, 10 aa, Homo sapiens
GVCGPSPPCI

SEQ ID NO: 411 - protein, 10 aa, Homo sapiens
VCGPSPPCIT

SEQ ID NO: 412 - protein, 10 aa, Homo sapiens
CGPSPPCITT

SEQ ID NO: 413 - protein, 10 aa, Homo sapiens
CAPIYCRRTC

SEQ ID NO: 414 - protein, 10 aa, Homo sapiens
CAPSPCQASC

SEQ ID NO: 415 - protein, 10 aa, Homo sapiens
CAPSPCQPAC

SEQ ID NO: 416 - protein, 10 aa, Homo sapiens
CAPVYCRRTC

SEQ ID NO: 417 - protein, 10 aa, Homo sapiens
CASSPCQQAC

SEQ ID NO: 418 - protein, 10 aa, Homo sapiens
CASSSCQPAC

SEQ ID NO: 419 - protein, 10 aa, Homo sapiens
CASSSCQQSC

SEQ ID NO: 420 - protein, 10 aa, Homo sapiens
CCGNFSSHSC

SEQ ID NO: 421 - protein, 10 aa, Homo sapiens
CCGYGGLGCG

SEQ ID NO: 422 - protein, 10 aa, Homo sapiens
CCNYYGNSCG

SEQ ID NO: 423 - protein, 10 aa, Homo sapiens
CCNYYRNSCG

SEQ ID NO: 424 - protein, 10 aa, Homo sapiens
CCSRNFSSCS

SEQ ID NO: 425 - protein, 10 aa, Homo sapiens
CDAGSCQPSC

SEQ ID NO: 426 - protein, 10 aa, Homo sapiens
CDPCSLQEGC

SEQ ID NO: 427 - protein, 10 aa, Homo sapiens
CDPSPCEPSC

SEQ ID NO: 428 - protein, 10 aa, Homo sapiens
CDPVICEPSC

SEQ ID NO: 429 - protein, 10 aa, Homo sapiens
CDQGLCQETC

SEQ ID NO: 430 - protein, 10 aa, Homo sapiens
CEATTCEPSC

SEQ ID NO: 431 - protein, 10 aa, Homo sapiens
CELPCGTPSC

SEQ ID NO: 432 - protein, 10 aa, Homo sapiens
CEPAICEPSC

SEQ ID NO: 433 - protein, 10 aa, Homo sapiens
CEPPCGTAPC

SEQ ID NO: 434 - protein, 10 aa, Homo sapiens
CEPPCSAPSC

SEQ ID NO: 435 - protein, 10 aa, Homo sapiens
CEPRSCASSC

SEQ ID NO: 436 - protein, 10 aa, Homo sapiens
CEPSACQSGC

SEQ ID NO: 437 - protein, 10 aa, Homo sapiens
CEPSCSVSNC

SEQ ID NO: 438 - protein, 10 aa, Homo sapiens
CEPSCSVSSC

SEQ ID NO: 439 - protein, 10 aa, Homo sapiens
CEPSPCQSGC

SEQ ID NO: 440 - protein, 10 aa, Homo sapiens
CEPTACQPTC

SEQ ID NO: 441 - protein, 10 aa, Homo sapiens
CEPTSCQTSC

SEQ ID NO: 442 - protein, 10 aa, Homo sapiens
CEPVCLRPVC

SEQ ID NO: 443 - protein, 10 aa, Homo sapiens
CETSSCQPRC

SEQ ID NO: 444 - protein, 10 aa, Homo sapiens
CETTCFQPTC

SEQ ID NO: 445 - protein, 10 aa, Homo sapiens
CFQPTCVSSC

SEQ ID NO: 446 - protein, 10 aa, Homo sapiens
CFQPTCVTSC

SEQ ID NO: 447 - protein, 10 aa, Homo sapiens
CFQPTCVYSC

SEQ ID NO: 448 - protein, 10 aa, Homo sapiens
CGCGFRRLGC

SEQ ID NO: 449 - protein, 10 aa, Homo sapiens
CGCGYRGLDC

SEQ ID NO: 450 - protein, 10 aa, Homo sapiens
CGCNGYYGCY

SEQ ID NO: 451 - protein, 10 aa, Homo sapiens
CGFGSCYGCG

SEQ ID NO: 452 - protein, 10 aa, Homo sapiens
CGGSGCGGSC

SEQ ID NO: 453 - protein, 10 aa, Homo sapiens
CGGSGSSCCV

SEQ ID NO: 454 - protein, 10 aa, Homo sapiens
CGGVSCHTTC

SEQ ID NO: 455 - protein, 10 aa, Homo sapiens
CGKGGCGSCG

SEQ ID NO: 456 - protein, 10 aa, Homo sapiens
CGKRGCGSCG

SEQ ID NO: 457 - protein, 10 aa, Homo sapiens
CGQDLCQETC

SEQ ID NO: 458 - protein, 10 aa, Homo sapiens
CGQTSCGSSC

SEQ ID NO: 459 - protein, 10 aa, Homo sapiens
CGQVLCQETC

SEQ ID NO: 460 - protein, 10 aa, Homo sapiens
CGRDLCQETC

SEQ ID NO: 461 - protein, 10 aa, Homo sapiens
CGRVSCHTTC

SEQ ID NO: 462 - protein, 10 aa, Homo sapiens
CGSCGFGSCY

SEQ ID NO: 463 - protein, 10 aa, Homo sapiens
CGSCGGSKGC

SEQ ID NO: 464 - protein, 10 aa, Homo sapiens
CGSGCGVPVC

SEQ ID NO: 465 - protein, 10 aa, Homo sapiens
CGSLLCRPTC

SEQ ID NO: 466 - protein, 10 aa, Homo sapiens
CGSRCYVPVC

SEQ ID NO: 467 - protein, 10 aa, Homo sapiens
CGSSSCGPQC

SEQ ID NO: 468 - protein, 10 aa, Homo sapiens
CGSVCSDQGC

SEQ ID NO: 469 - protein, 10 aa, Homo sapiens
CGSVCSDQSC

SEQ ID NO: 470 - protein, 10 aa, Homo sapiens
CGSVCSHQGC

SEQ ID NO: 471 - protein, 10 aa, Homo sapiens
CGSYGCSQCS

SEQ ID NO: 472 - protein, 10 aa, Homo sapiens
CGVCLPSTCP

SEQ ID NO: 473 - protein, 10 aa, Homo sapiens
CGYEGCRYGC

SEQ ID NO: 474 - protein, 10 aa, Homo sapiens
CGYGCGYGCG

SEQ ID NO: 475 - protein, 10 aa, Homo sapiens
CGYGGCGYGC

SEQ ID NO: 476 - protein, 10 aa, Homo sapiens
CGYGSFCGCG

SEQ ID NO: 477 - protein, 10 aa, Homo sapiens
CGYGSGCGCG

SEQ ID NO: 478 - protein, 10 aa, Homo sapiens
CHPSCGMSSC

SEQ ID NO: 479 - protein, 10 aa, Homo sapiens
CHPSCSISSC

SEQ ID NO: 480 - protein, 10 aa, Homo sapiens
CHPTCYQTIC

SEQ ID NO: 481 - protein, 10 aa, Homo sapiens
CHTSCSPACQ

SEQ ID NO: 482 - protein, 10 aa, Homo sapiens
CHTSCSSGCQ

SEQ ID NO: 483 - protein, 10 aa, Homo sapiens
CHTTCYRPAC

SEQ ID NO: 484 - protein, 10 aa, Homo sapiens
CHTTCYRPTC

SEQ ID NO: 485 - protein, 10 aa, Homo sapiens
CIHSPCQASC

SEQ ID NO: 486 - protein, 10 aa, Homo sapiens
CIHSTHCGCN

SEQ ID NO: 487 - protein, 10 aa, Homo sapiens
CIRSPCQASC

SEQ ID NO: 488 - protein, 10 aa, Homo sapiens
CISSCYRPQC

SEQ ID NO: 489 - protein, 10 aa, Homo sapiens
CISSPCQQSC

SEQ ID NO: 490 - protein, 10 aa, Homo sapiens
CKPCSSQSSC

SEQ ID NO: 491 - protein, 10 aa, Homo sapiens
CKPSCSQSSC

SEQ ID NO: 492 - protein, 10 aa, Homo sapiens
CKPVCFKPIC

SEQ ID NO: 493 - protein, 10 aa, Homo sapiens
CKPVCYVPTC

SEQ ID NO: 494 - protein, 10 aa, Homo sapiens
CKPVSCVPVC

SEQ ID NO: 495 - protein, 10 aa, Homo sapiens
CKPVYCVPVC

SEQ ID NO: 496 - protein, 10 aa, Homo sapiens
CKTVYCKPIC

SEQ ID NO: 497 - protein, 10 aa, Homo sapiens
CLNQSCGSNC

SEQ ID NO: 498 - protein, 10 aa, Homo sapiens
CLNQSCGSSC

SEQ ID NO: 499 - protein, 10 aa, Homo sapiens
CLPGCLNQSC

SEQ ID NO: 500 - protein, 10 aa, Homo sapiens
CLPGSCDSCS

SEQ ID NO: 501 - protein, 10 aa, Homo sapiens
CLPPCYLVSC

SEQ ID NO: 502 - protein, 10 aa, Homo sapiens
CLPTSCQPSC

SEQ ID NO: 503 - protein, 10 aa, Homo sapiens
CLSFLCRPAC

SEQ ID NO: 504 - protein, 10 aa, Homo sapiens
CLVSSCQPSC

SEQ ID NO: 505 - protein, 10 aa, Homo sapiens
CMPSPCQPAC

SEQ ID NO: 506 - protein, 10 aa, Homo sapiens
CMSGSCQAAC

SEQ ID NO: 507 - protein, 10 aa, Homo sapiens
CNESSYCLPC

SEQ ID NO: 508 - protein, 10 aa, Homo sapiens
CPASCVSLLC

SEQ ID NO: 509 - protein, 10 aa, Homo sapiens
CPMACPGSPC

SEQ ID NO: 510 - protein, 10 aa, Homo sapiens
CPSSCTAVVC

SEQ ID NO: 511 - protein, 10 aa, Homo sapiens
CPVTCEPSPC

SEQ ID NO: 512 - protein, 10 aa, Homo sapiens
CQAACEPSAC

SEQ ID NO: 513 - protein, 10 aa, Homo sapiens
CQAACEPSPC

SEQ ID NO: 514 - protein, 10 aa, Homo sapiens
CQAACGQSVC

SEQ ID NO: 515 - protein, 10 aa, Homo sapiens
CQAPCSTKNC

SEQ ID NO: 516 - protein, 10 aa, Homo sapiens
CQAVCEPSPC

SEQ ID NO: 517 - protein, 10 aa, Homo sapiens
CQDSCGSSSC

SEQ ID NO: 518 - protein, 10 aa, Homo sapiens
CQHSSCQPTC

SEQ ID NO: 519 - protein, 10 aa, Homo sapiens
CQISSCGTGC

SEQ ID NO: 520 - protein, 10 aa, Homo sapiens
CQKSSCQPAC

SEQ ID NO: 521 - protein, 10 aa, Homo sapiens
CQPMCSHAAC

SEQ ID NO: 522 - protein, 10 aa, Homo sapiens
CQPPCTTALC

SEQ ID NO: 523 - protein, 10 aa, Homo sapiens
CQPSCESSFC

SEQ ID NO: 524 - protein, 10 aa, Homo sapiens
CQPSCSESTC

SEQ ID NO: 525 - protein, 10 aa, Homo sapiens
CQPSCTSVLC

SEQ ID NO: 526 - protein, 10 aa, Homo sapiens
CQPTCGGSSC

SEQ ID NO: 527 - protein, 10 aa, Homo sapiens
CQPTCSRPSC

SEQ ID NO: 528 - protein, 10 aa, Homo sapiens
CQPVCPTPTC

SEQ ID NO: 529 - protein, 10 aa, Homo sapiens
CQPVLCKSSC

SEQ ID NO: 530 - protein, 10 aa, Homo sapiens
CQPVVCEPSC

SEQ ID NO: 531 - protein, 10 aa, Homo sapiens
CQQPSCQPAC

SEQ ID NO: 532 - protein, 10 aa, Homo sapiens
CQQSCRVPVC

SEQ ID NO: 533 - protein, 10 aa, Homo sapiens
CQQSCYVPVC

SEQ ID NO: 534 - protein, 10 aa, Homo sapiens
CQQSGCQPAC

SEQ ID NO: 535 - protein, 10 aa, Homo sapiens
CQQSSCHPAC

SEQ ID NO: 536 - protein, 10 aa, Homo sapiens
CQQSSCKPAC

SEQ ID NO: 537 - protein, 10 aa, Homo sapiens
CQQSSCQLAC

SEQ ID NO: 538 - protein, 10 aa, Homo sapiens
CQQSSCQPAC

SEQ ID NO: 539 - protein, 10 aa, Homo sapiens
CQQSSCQPTC

SEQ ID NO: 540 - protein, 10 aa, Homo sapiens
CQQSSCQSAC

SEQ ID NO: 541 - protein, 10 aa, Homo sapiens
CQQSSCVSCV

SEQ ID NO: 542 - protein, 10 aa, Homo sapiens
CQQSYCVPVC

SEQ ID NO: 543 - protein, 10 aa, Homo sapiens
CQSGCISSCT

SEQ ID NO: 544 - protein, 10 aa, Homo sapiens
CQSGCTDSCT

SEQ ID NO: 545 - protein, 10 aa, Homo sapiens
CQSGCTSSCT

SEQ ID NO: 546 - protein, 10 aa, Homo sapiens
CQSSCYRPTC

SEQ ID NO: 547 - protein, 10 aa, Homo sapiens
CQSVCYQPTC

SEQ ID NO: 548 - protein, 10 aa, Homo sapiens
CQSVYCQPTC

SEQ ID NO: 549 - protein, 10 aa, Homo sapiens
CQTACEPSAC

SEQ ID NO: 550 - protein, 10 aa, Homo sapiens
CQTSSCGTGC

SEQ ID NO: 551 - protein, 10 aa, Homo sapiens
CQTTCHPSCG

SEQ ID NO: 552 - protein, 10 aa, Homo sapiens
CQTTCRPSCG

SEQ ID NO: 553 - protein, 10 aa, Homo sapiens
CQTTCYRTTC

SEQ ID NO: 554 - protein, 10 aa, Homo sapiens
CQTTRCRTTC

SEQ ID NO: 555 - protein, 10 aa, Homo sapiens
CQVTCEPSPC

SEQ ID NO: 556 - protein, 10 aa, Homo sapiens
CRNTSCQPTC

SEQ ID NO: 557 - protein, 10 aa, Homo sapiens
CRPACSPLAC

SEQ ID NO: 558 - protein, 10 aa, Homo sapiens
CRPACSRLAC

SEQ ID NO: 559 - protein, 10 aa, Homo sapiens
CRPACSRPAC

SEQ ID NO: 560 - protein, 10 aa, Homo sapiens
CRPMCSRPAC

SEQ ID NO: 561 - protein, 10 aa, Homo sapiens
CRPSCGQTTC

SEQ ID NO: 562 - protein, 10 aa, Homo sapiens
CRPSCGVSSC

SEQ ID NO: 563 - protein, 10 aa, Homo sapiens
CRPSCSISSC

SEQ ID NO: 564 - protein, 10 aa, Homo sapiens
CRPSCSQTTC

SEQ ID NO: 565 - protein, 10 aa, Homo sapiens
CRPSYCGQSC

SEQ ID NO: 566 - protein, 10 aa, Homo sapiens
CRPSYCISSC

SEQ ID NO: 567 - protein, 10 aa, Homo sapiens
CRPSYCQTTC

SEQ ID NO: 568 - protein, 10 aa, Homo sapiens
CRPTCSRLAC

SEQ ID NO: 569 - protein, 10 aa, Homo sapiens
CRPTCSSGSC

SEQ ID NO: 570 - protein, 10 aa, Homo sapiens
CRPTSCQNTC

SEQ ID NO: 571 - protein, 10 aa, Homo sapiens
CRPVCRSTYC

SEQ ID NO: 572 - protein, 10 aa, Homo sapiens
CRPVCSRPAC

SEQ ID NO: 573 - protein, 10 aa, Homo sapiens
CRPVTCVPRC

SEQ ID NO: 574 - protein, 10 aa, Homo sapiens
CRQSSCQPAC

SEQ ID NO: 575 - protein, 10 aa, Homo sapiens
CRTTCFHPIC

SEQ ID NO: 576 - protein, 10 aa, Homo sapiens
CRTTCFQPTC

SEQ ID NO: 577 - protein, 10 aa, Homo sapiens
CRTTCYRPSC

SEQ ID NO: 578 - protein, 10 aa, Homo sapiens
CRTTYCRPSC

SEQ ID NO: 579 - protein, 10 aa, Homo sapiens
CRVTCEPSPC

SEQ ID NO: 580 - protein, 10 aa, Homo sapiens
CRYGCGHRGC

SEQ ID NO: 581 - protein, 10 aa, Homo sapiens
CSAPCVALLC

SEQ ID NO: 582 - protein, 10 aa, Homo sapiens
CSDDSGSCCQ

SEQ ID NO: 583 - protein, 10 aa, Homo sapiens
CSEDSSSCCQ

SEQ ID NO: 584 - protein, 10 aa, Homo sapiens
CSEDSYSCCQ

SEQ ID NO: 585 - protein, 10 aa, Homo sapiens
CSEGCGSGCG

SEQ ID NO: 586 - protein, 10 aa, Homo sapiens
CSESSPSCCQ

SEQ ID NO: 587 - protein, 10 aa, Homo sapiens
CSESSSSCCQ

SEQ ID NO: 588 - protein, 10 aa, Homo sapiens
CSFDKSCRCG

SEQ ID NO: 589 - protein, 10 aa, Homo sapiens
CSGASSLCCQ

SEQ ID NO: 590 - protein, 10 aa, Homo sapiens
CSGASSPCCQ

SEQ ID NO: 591 - protein, 10 aa, Homo sapiens
CSGASSSCCQ

SEQ ID NO: 592 - protein, 10 aa, Homo sapiens
CSGASTSCCQ

SEQ ID NO: 593 - protein, 10 aa, Homo sapiens
CSGGCGSGCG

SEQ ID NO: 594 - protein, 10 aa, Homo sapiens
CSGGCGSSCG

SEQ ID NO: 595 - protein, 10 aa, Homo sapiens
CSGISSSCCQ

SEQ ID NO: 596 - protein, 10 aa, Homo sapiens
CSKDSSSCCQ

SEQ ID NO: 597 - protein, 10 aa, Homo sapiens
CSKGACGSCG

SEQ ID NO: 598 - protein, 10 aa, Homo sapiens
CSLSCGSRSC

SEQ ID NO: 599 - protein, 10 aa, Homo sapiens
CSQDLCQETC

SEQ ID NO: 600 - protein, 10 aa, Homo sapiens
CSRGCGSGCG

SEQ ID NO: 601 - protein, 10 aa, Homo sapiens
CSRLSSACCG

SEQ ID NO: 602 - protein, 10 aa, Homo sapiens
CSSCGKGGCG

SEQ ID NO: 603 - protein, 10 aa, Homo sapiens
CSSCGKRGCG

SEQ ID NO: 604 - protein, 10 aa, Homo sapiens
CSSDKSCRCG

SEQ ID NO: 605 - protein, 10 aa, Homo sapiens
CSSGNFSSCC

SEQ ID NO: 606 - protein, 10 aa, Homo sapiens
CSSSGCGSFC

SEQ ID NO: 607 - protein, 10 aa, Homo sapiens
CSSSGCGSSC

SEQ ID NO: 608 - protein, 10 aa, Homo sapiens
CSTPCYQPIC

SEQ ID NO: 609 - protein, 10 aa, Homo sapiens
CSTTCRTSSC

SEQ ID NO: 610 - protein, 10 aa, Homo sapiens
CSWVPACSCT

SEQ ID NO: 611 - protein, 10 aa, Homo sapiens
CTFSPCQQAC

SEQ ID NO: 612 - protein, 10 aa, Homo sapiens
CTMSVCSSAC

SEQ ID NO: 613 - protein, 10 aa, Homo sapiens
CTRPICEPCR

SEQ ID NO: 614 - protein, 10 aa, Homo sapiens
CTSSPCQHAC

SEQ ID NO: 615 - protein, 10 aa, Homo sapiens
CTSSPCQQAC

SEQ ID NO: 616 - protein, 10 aa, Homo sapiens
CTSSPCQQSC

SEQ ID NO: 617 - protein, 10 aa, Homo sapiens
CTSSSCQQAC

SEQ ID NO: 618 - protein, 10 aa, Homo sapiens
CVALLCRPLC

SEQ ID NO: 619 - protein, 10 aa, Homo sapiens
CVALVCEPVC

SEQ ID NO: 620 - protein, 10 aa, Homo sapiens
CVFSSCNTTC

SEQ ID NO: 621 - protein, 10 aa, Homo sapiens
CVGFVCQPMC

SEQ ID NO: 622 - protein, 10 aa, Homo sapiens
CVPRCTRPIC

SEQ ID NO: 623 - protein, 10 aa, Homo sapiens
CVPSPCQVAC

SEQ ID NO: 624 - protein, 10 aa, Homo sapiens
CVPSRCQASC

SEQ ID NO: 625 - protein, 10 aa, Homo sapiens
CVPSSCQASC

SEQ ID NO: 626 - protein, 10 aa, Homo sapiens
CVPVCNKPVC

SEQ ID NO: 627 - protein, 10 aa, Homo sapiens
CVPVCSKSVC

SEQ ID NO: 628 - protein, 10 aa, Homo sapiens
CVPVRCKPVC

SEQ ID NO: 629 - protein, 10 aa, Homo sapiens
CVSLLCRPAC

SEQ ID NO: 630 - protein, 10 aa, Homo sapiens
CVSLLCRPMC

SEQ ID NO: 631 - protein, 10 aa, Homo sapiens
CVSLLCRPTC

SEQ ID NO: 632 - protein, 10 aa, Homo sapiens
CVSLLCRPVC

SEQ ID NO: 633 - protein, 10 aa, Homo sapiens
CVSNPCQVTC

SEQ ID NO: 634 - protein, 10 aa, Homo sapiens
CVSRCYRPHC

SEQ ID NO: 635 - protein, 10 aa, Homo sapiens
CVSSCFRPQC

SEQ ID NO: 636 - protein, 10 aa, Homo sapiens
CVSSICQPIC

SEQ ID NO: 637 - protein, 10 aa, Homo sapiens
CVSSPCQPTC

SEQ ID NO: 638 - protein, 10 aa, Homo sapiens
CVVSCTPPSC

SEQ ID NO: 639 - protein, 10 aa, Homo sapiens
CVVSCTPPTC

SEQ ID NO: 640 - protein, 10 aa, Homo sapiens
CYCPKNSIFC

SEQ ID NO: 641 - protein, 10 aa, Homo sapiens
CYEPCLPRGC

SEQ ID NO: 642 - protein, 10 aa, Homo sapiens
CYRRCYSSCY

SEQ ID NO: 643 - protein, 10 aa, Homo sapiens
GCCGYGGLGC

SEQ ID NO: 644 - protein, 10 aa, Homo sapiens
GCGGCGSGCA

SEQ ID NO: 645 - protein, 10 aa, Homo sapiens
GCGGCGSGCG

SEQ ID NO: 646 - protein, 10 aa, Homo sapiens
GCGGCGSSCG

SEQ ID NO: 647 - protein, 10 aa, Homo sapiens
GCGGCSSSCG

SEQ ID NO: 648 - protein, 10 aa, Homo sapiens
GCGGSGSSCC

SEQ ID NO: 649 - protein, 10 aa, Homo sapiens
GCGSGCAGCG

SEQ ID NO: 650 - protein, 10 aa, Homo sapiens
GCGSGCGGCG

SEQ ID NO: 651 - protein, 10 aa, Homo sapiens
GCGSGCGGCS

SEQ ID NO: 652 - protein, 10 aa, Homo sapiens
GCGSSCGGCD

SEQ ID NO: 653 - protein, 10 aa, Homo sapiens
GCGSSCGGCG

SEQ ID NO: 654 - protein, 10 aa, Homo sapiens
GCGSSCSQCS

SEQ ID NO: 655 - protein, 10 aa, Homo sapiens
GCGYSSSCCG

SEQ ID NO: 656 - protein, 10 aa, Homo sapiens
GCKGGCGSCG

SEQ ID NO: 657 - protein, 10 aa, Homo sapiens
GCSGCSGGCG

SEQ ID NO: 658 - protein, 10 aa, Homo sapiens
ICSGASSLCC

SEQ ID NO: 659 - protein, 10 aa, Homo sapiens
ICSGASSPCC

SEQ ID NO: 660 - protein, 10 aa, Homo sapiens
MCCNYYGNSC

SEQ ID NO: 661 - protein, 10 aa, Homo sapiens
MCCNYYRNSC

SEQ ID NO: 662 - protein, 10 aa, Homo sapiens
MCYGYGCGCG

SEQ ID NO: 663 - protein, 10 aa, Homo sapiens
NCCSRNFSSC

SEQ ID NO: 664 - protein, 10 aa, Homo sapiens
PCSLQEGCCR

SEQ ID NO: 665 - protein, 10 aa, Homo sapiens
PCSSQSSCCV

SEQ ID NO: 666 - protein, 10 aa, Homo sapiens
SCCAPASSCQ

SEQ ID NO: 667 - protein, 10 aa, Homo sapiens
SCCAPASTCQ

SEQ ID NO: 668 - protein, 10 aa, Homo sapiens
SCCAPTSSCQ

SEQ ID NO: 669 - protein, 10 aa, Homo sapiens
SCCGYRPLCY

SEQ ID NO: 670 - protein, 10 aa, Homo sapiens
SCCVPASSCQ

SEQ ID NO: 671 - protein, 10 aa, Homo sapiens
SCCVPTSSCQ

SEQ ID NO: 672 - protein, 10 aa, Homo sapiens
SCGCSKGACG

SEQ ID NO: 673 - protein, 10 aa, Homo sapiens
SCGGCDSSCG

SEQ ID NO: 674 - protein, 10 aa, Homo sapiens
SCGGCGSGCG

SEQ ID NO: 675 - protein, 10 aa, Homo sapiens
SCGGCGSSCG

SEQ ID NO: 676 - protein, 10 aa, Homo sapiens
SCGGCKGGCG

SEQ ID NO: 677 - protein, 10 aa, Homo sapiens
SCGGSKGCCG

SEQ ID NO: 678 - protein, 10 aa, Homo sapiens
SCGSGCRGCG

SEQ ID NO: 679 - protein, 10 aa, Homo sapiens
SCYGCGYGCI

SEQ ID NO: 680 - protein, 10 aa, Homo sapiens
TCCVPVPSCG

SEQ ID NO: 681 - protein, 10 aa, Homo sapiens
TCSDDSGSCC

SEQ ID NO: 682 - protein, 10 aa, Homo sapiens
TCSEDSSSCC

SEQ ID NO: 683 - protein, 10 aa, Homo sapiens
TCSEDSYSCC

SEQ ID NO: 684 - protein, 10 aa, Homo sapiens
TCSESSPSCC

SEQ ID NO: 685 - protein, 10 aa, Homo sapiens
TCSESSSSCC

SEQ ID NO: 686 - protein, 10 aa, Homo sapiens
TCSKDSSSCC

SEQ ID NO: 687 - protein, 10 aa, Homo sapiens
TCSRLSSACC

SEQ ID NO: 688 - protein, 10 aa, Homo sapiens
VCCQPTPICD

SEQ ID NO: 689 - protein, 10 aa, Homo sapiens
VCSEDSSSCC

SEQ ID NO: 690 - protein, 10 aa, Homo sapiens
VCSGASSLCC

SEQ ID NO: 691 - protein, 10 aa, Homo sapiens
VCSGASSPCC

SEQ ID NO: 692 - protein, 10 aa, Homo sapiens
VCSGASSSCC

SEQ ID NO: 693 - protein, 10 aa, Homo sapiens
VCSGASTSCC

SEQ ID NO: 694 - protein, 10 aa, Homo sapiens
VCSGDSSCCQ

SEQ ID NO: 695 - protein, 10 aa, Homo sapiens
VCSGISSSCC

SEQ ID NO: 696 - protein, 10 aa, Homo sapiens
YCVPIPSCCA

SEQ ID NO: 697 - protein, 10 aa, Homo sapiens
CASSCCTPSC

SEQ ID NO: 698 - protein, 10 aa, Homo sapiens
CCDNCPPPCH

SEQ ID NO: 699 - protein, 10 aa, Homo sapiens
CCEPCLPRGC

SEQ ID NO: 700 - protein, 10 aa, Homo sapiens
CCGAASSCCR

SEQ ID NO: 701 - protein, 10 aa, Homo sapiens
CCGCGGSGCG

SEQ ID NO: 702 - protein, 10 aa, Homo sapiens
CCGPSSSCCQ

SEQ ID NO: 703 - protein, 10 aa, Homo sapiens
CCGSGCGGCG

SEQ ID NO: 704 - protein, 10 aa, Homo sapiens
CCKPYCSQCS

SEQ ID NO: 705 - protein, 10 aa, Homo sapiens
CCMPVSSCCA

SEQ ID NO: 706 - protein, 10 aa, Homo sapiens
CCNYYRNCCG

SEQ ID NO: 707 - protein, 10 aa, Homo sapiens
CCPSCVVSSC

SEQ ID NO: 708 - protein, 10 aa, Homo sapiens
CCPSYCVSSC

SEQ ID NO: 709 - protein, 10 aa, Homo sapiens
CCQPICGSSC

SEQ ID NO: 710 - protein, 10 aa, Homo sapiens
CCQPICVTSC

SEQ ID NO: 711 - protein, 10 aa, Homo sapiens
CCQPTCLSSC

SEQ ID NO: 712 - protein, 10 aa, Homo sapiens
CCQPTCLTSC

SEQ ID NO: 713 - protein, 10 aa, Homo sapiens
CCQPTCVASC

SEQ ID NO: 714 - protein, 10 aa, Homo sapiens
CCQPTCVTSC

SEQ ID NO: 715 - protein, 10 aa, Homo sapiens
CCQPYCHPTC

SEQ ID NO: 716 - protein, 10 aa, Homo sapiens
CCQQSSCVSC

SEQ ID NO: 717 - protein, 10 aa, Homo sapiens
CCQSSCFKPC

SEQ ID NO: 718 - protein, 10 aa, Homo sapiens
CCQSSCSKPC

SEQ ID NO: 719 - protein, 10 aa, Homo sapiens
CCQSSCYKPC

SEQ ID NO: 720 - protein, 10 aa, Homo sapiens
CCQTICRSTC

SEQ ID NO: 721 - protein, 10 aa, Homo sapiens
CCQTTCHPSC

SEQ ID NO: 722 - protein, 10 aa, Homo sapiens
CCQTTCRPSC

SEQ ID NO: 723 - protein, 10 aa, Homo sapiens
CCRVPTCSCS

SEQ ID NO: 724 - protein, 10 aa, Homo sapiens
CCSPGCQPTC

SEQ ID NO: 725 - protein, 10 aa, Homo sapiens
CCSSGCGSSC

SEQ ID NO: 726 - protein, 10 aa, Homo sapiens
CCSSSCGSCG

SEQ ID NO: 727 - protein, 10 aa, Homo sapiens
CCTQEQNCCE

SEQ ID NO: 728 - protein, 10 aa, Homo sapiens
CCVPIPSCCA

SEQ ID NO: 729 - protein, 10 aa, Homo sapiens
CCVPISSCCA

SEQ ID NO: 730 - protein, 10 aa, Homo sapiens
CCVPVCYQCK

SEQ ID NO: 731 - protein, 10 aa, Homo sapiens
CCVPVPSCCA

SEQ ID NO: 732 - protein, 10 aa, Homo sapiens
CCVPVPSCCV

SEQ ID NO: 733 - protein, 10 aa, Homo sapiens
CCVPVSSCCA

SEQ ID NO: 734 - protein, 10 aa, Homo sapiens
CDSSCCQPSC

SEQ ID NO: 735 - protein, 10 aa, Homo sapiens
CDTCPPPCCK

SEQ ID NO: 736 - protein, 10 aa, Homo sapiens
CEPCRRPVCC

SEQ ID NO: 737 - protein, 10 aa, Homo sapiens
CEPSCCQPVC

SEQ ID NO: 738 - protein, 10 aa, Homo sapiens
CEPSCCSAVC

SEQ ID NO: 739 - protein, 10 aa, Homo sapiens
CETSCCQPSC

SEQ ID NO: 740 - protein, 10 aa, Homo sapiens
CETTCCRTTC

SEQ ID NO: 741 - protein, 10 aa, Homo sapiens
CFSGCGSSCC

SEQ ID NO: 742 - protein, 10 aa, Homo sapiens
CGCSQSNCCK

SEQ ID NO: 743 - protein, 10 aa, Homo sapiens
CGCSQSSCCK

SEQ ID NO: 744 - protein, 10 aa, Homo sapiens
CGGCGGCGGC

SEQ ID NO: 745 - protein, 10 aa, Homo sapiens
CGGCGGGCCG

SEQ ID NO: 746 - protein, 10 aa, Homo sapiens
CGGCGSGCCV

SEQ ID NO: 747 - protein, 10 aa, Homo sapiens
CGGCGSSCCV

SEQ ID NO: 748 - protein, 10 aa, Homo sapiens
CGGGCCGSSC

SEQ ID NO: 749 - protein, 10 aa, Homo sapiens
CGGSCCGSSC

SEQ ID NO: 750 - protein, 10 aa, Homo sapiens
CGQSCCRPAC

SEQ ID NO: 751 - protein, 10 aa, Homo sapiens
CGQSCCRPVC

SEQ ID NO: 752 - protein, 10 aa, Homo sapiens
CGSCGCSQCN

SEQ ID NO: 753 - protein, 10 aa, Homo sapiens
CGSCGCSQCS

SEQ ID NO: 754 - protein, 10 aa, Homo sapiens
CGSFCCQSSC

SEQ ID NO: 755 - protein, 10 aa, Homo sapiens
CGSGCCVPVC

SEQ ID NO: 756 - protein, 10 aa, Homo sapiens
CGSSCCGSGC

SEQ ID NO: 757 - protein, 10 aa, Homo sapiens
CGSSCCQPCY

SEQ ID NO: 758 - protein, 10 aa, Homo sapiens
CGSSCCQPIC

SEQ ID NO: 759 - protein, 10 aa, Homo sapiens
CGSSCCQPSC

SEQ ID NO: 760 - protein, 10 aa, Homo sapiens
CGSSCCQSSC

SEQ ID NO: 761 - protein, 10 aa, Homo sapiens
CGSSCCVPIC

SEQ ID NO: 762 - protein, 10 aa, Homo sapiens
CGSSCCVPVC

SEQ ID NO: 763 - protein, 10 aa, Homo sapiens
CGSSCSQCSC

SEQ ID NO: 764 - protein, 10 aa, Homo sapiens
CGYGSCCGCG

SEQ ID NO: 765 - protein, 10 aa, Homo sapiens
CHPRCCISSC

SEQ ID NO: 766 - protein, 10 aa, Homo sapiens
CHPSCCESSC

SEQ ID NO: 767 - protein, 10 aa, Homo sapiens
CHPSCCISSC

SEQ ID NO: 768 - protein, 10 aa, Homo sapiens
CHPTCCQNTC

SEQ ID NO: 769 - protein, 10 aa, Homo sapiens
CHPTCCQTIC

SEQ ID NO: 770 - protein, 10 aa, Homo sapiens
CHPVCCQTTC

SEQ ID NO: 771 - protein, 10 aa, Homo sapiens
CHPVCKSTCC

SEQ ID NO: 772 - protein, 10 aa, Homo sapiens
CHPVCRSTCC

SEQ ID NO: 773 - protein, 10 aa, Homo sapiens
CISSCCHPSC

SEQ ID NO: 774 - protein, 10 aa, Homo sapiens
CISSCCKPSC

SEQ ID NO: 775 - protein, 10 aa, Homo sapiens
CISSCCRPSC

SEQ ID NO: 776 - protein, 10 aa, Homo sapiens
CISSCTPSCC

SEQ ID NO: 777 - protein, 10 aa, Homo sapiens
CISSSCCPSC

SEQ ID NO: 778 - protein, 10 aa, Homo sapiens
CKAVCCVPTC

SEQ ID NO: 779 - protein, 10 aa, Homo sapiens
CKPCCSQASC

SEQ ID NO: 780 - protein, 10 aa, Homo sapiens
CKPCCSQSRC

SEQ ID NO: 781 - protein, 10 aa, Homo sapiens
CKPCCSQSSC

SEQ ID NO: 782 - protein, 10 aa, Homo sapiens
CKPCCSSSGC

SEQ ID NO: 783 - protein, 10 aa, Homo sapiens
CKPCSCFSGC

SEQ ID NO: 784 - protein, 10 aa, Homo sapiens
CKPCSCSSGC

SEQ ID NO: 785 - protein, 10 aa, Homo sapiens
CKPCYCSSGC

SEQ ID NO: 786 - protein, 10 aa, Homo sapiens
CKPICCVPVC

SEQ ID NO: 787 - protein, 10 aa, Homo sapiens
CKPQCCQSVC

SEQ ID NO: 788 - protein, 10 aa, Homo sapiens
CKPSCCQTTC

SEQ ID NO: 789 - protein, 10 aa, Homo sapiens
CKPVCCAPTC

SEQ ID NO: 790 - protein, 10 aa, Homo sapiens
CKPVCCKPIC

SEQ ID NO: 791 - protein, 10 aa, Homo sapiens
CKPVCCKSIC

SEQ ID NO: 792 - protein, 10 aa, Homo sapiens
CKPVCCLPTC

SEQ ID NO: 793 - protein, 10 aa, Homo sapiens
CKPVCCVPTC

SEQ ID NO: 794 - protein, 10 aa, Homo sapiens
CKPVCCVPVC

SEQ ID NO: 795 - protein, 10 aa, Homo sapiens
CKPVCCVSTC

SEQ ID NO: 796 - protein, 10 aa, Homo sapiens
CKPYCCQSSC

SEQ ID NO: 797 - protein, 10 aa, Homo sapiens
CKPYCSQCSC

SEQ ID NO: 798 - protein, 10 aa, Homo sapiens
CKSNCCKPVC

SEQ ID NO: 799 - protein, 10 aa, Homo sapiens
CKTVCCKPVC

SEQ ID NO: 800 - protein, 10 aa, Homo sapiens
CLPPCCVVSC

SEQ ID NO: 801 - protein, 10 aa, Homo sapiens
CLTSCCQPSC

SEQ ID NO: 802 - protein, 10 aa, Homo sapiens
CNPCCSQSSC

SEQ ID NO: 803 - protein, 10 aa, Homo sapiens
CPESCCELPC

SEQ ID NO: 804 - protein, 10 aa, Homo sapiens
CPESCCEPHC

SEQ ID NO: 805 - protein, 10 aa, Homo sapiens
CPESCCEPPC

SEQ ID NO: 806 - protein, 10 aa, Homo sapiens
CPFSCPTTCC

SEQ ID NO: 807 - protein, 10 aa, Homo sapiens
CPGDCFTCCT

SEQ ID NO: 808 - protein, 10 aa, Homo sapiens
CPSCVVSSCC

SEQ ID NO: 809 - protein, 10 aa, Homo sapiens
CPSYCVSSCC

SEQ ID NO: 810 - protein, 10 aa, Homo sapiens
CPTTCCRTTC

SEQ ID NO: 811 - protein, 10 aa, Homo sapiens
CQETCCRPSC

SEQ ID NO: 812 - protein, 10 aa, Homo sapiens
CQHACCVPVC

SEQ ID NO: 813 - protein, 10 aa, Homo sapiens
CQNTCCRTTC

SEQ ID NO: 814 - protein, 10 aa, Homo sapiens
CQPACCQPTC

SEQ ID NO: 815 - protein, 10 aa, Homo sapiens
CQPACCTASC

SEQ ID NO: 816 - protein, 10 aa, Homo sapiens
CQPACCTSSC

SEQ ID NO: 817 - protein, 10 aa, Homo sapiens
CQPACCTTSC

SEQ ID NO: 818 - protein, 10 aa, Homo sapiens
CQPACCVPVC

SEQ ID NO: 819 - protein, 10 aa, Homo sapiens
CQPACCVSSC

SEQ ID NO: 820 - protein, 10 aa, Homo sapiens
CQPCCHPTCY

SEQ ID NO: 821 - protein, 10 aa, Homo sapiens
CQPCCRPTSC

SEQ ID NO: 822 - protein, 10 aa, Homo sapiens
CQPICCGSSC

SEQ ID NO: 823 - protein, 10 aa, Homo sapiens
CQPICGSSCC

SEQ ID NO: 824 - protein, 10 aa, Homo sapiens
CQPICVTSCC

SEQ ID NO: 825 - protein, 10 aa, Homo sapiens
CQPNCCRPSC

SEQ ID NO: 826 - protein, 10 aa, Homo sapiens
CQPRCCETSC

SEQ ID NO: 827 - protein, 10 aa, Homo sapiens
CQPSCCRPAC

SEQ ID NO: 828 - protein, 10 aa, Homo sapiens
CQPSCCSTPC

SEQ ID NO: 829 - protein, 10 aa, Homo sapiens
CQPSCCSTTC

SEQ ID NO: 830 - protein, 10 aa, Homo sapiens
CQPSCCVPSC

SEQ ID NO: 831 - protein, 10 aa, Homo sapiens
CQPSCCVSSC

SEQ ID NO: 832 - protein, 10 aa, Homo sapiens
CQPTCCGSSC

SEQ ID NO: 833 - protein, 10 aa, Homo sapiens
CQPTCCHPSC

SEQ ID NO: 834 - protein, 10 aa, Homo sapiens
CQPTCCQPTC

SEQ ID NO: 835 - protein, 10 aa, Homo sapiens
CQPTCCRPRC

SEQ ID NO: 836 - protein, 10 aa, Homo sapiens
CQPTCCRPSC

SEQ ID NO: 837 - protein, 10 aa, Homo sapiens
CQPTCCRTTC

SEQ ID NO: 838 - protein, 10 aa, Homo sapiens
CQPTCLSSCC

SEQ ID NO: 839 - protein, 10 aa, Homo sapiens
CQPTCLTSCC

SEQ ID NO: 840 - protein, 10 aa, Homo sapiens
CQPTCVASCC

SEQ ID NO: 841 - protein, 10 aa, Homo sapiens
CQPTCVTSCC

SEQ ID NO: 842 - protein, 10 aa, Homo sapiens
CQPVCCQPTC

SEQ ID NO: 843 - protein, 10 aa, Homo sapiens
CQPYCHPTCC

SEQ ID NO: 844 - protein, 10 aa, Homo sapiens
CQQACCMPVC

SEQ ID NO: 845 - protein, 10 aa, Homo sapiens
CQQACCVPIC

SEQ ID NO: 846 - protein, 10 aa, Homo sapiens
CQQACCVPVC

SEQ ID NO: 847 - protein, 10 aa, Homo sapiens
CQQSCCVPVC

SEQ ID NO: 848 - protein, 10 aa, Homo sapiens
CQQSCCVSVC

SEQ ID NO: 849 - protein, 10 aa, Homo sapiens
CQSMCCQPTC

SEQ ID NO: 850 - protein, 10 aa, Homo sapiens
CQSNCCVPVC

SEQ ID NO: 851 - protein, 10 aa, Homo sapiens
CQSSCCKPCS

SEQ ID NO: 852 - protein, 10 aa, Homo sapiens
CQSSCCQSSC

SEQ ID NO: 853 - protein, 10 aa, Homo sapiens
CQSSCCVPVC

SEQ ID NO: 854 - protein, 10 aa, Homo sapiens
CQSSCFKPCC

SEQ ID NO: 855 - protein, 10 aa, Homo sapiens
CQSSCSKPCC

SEQ ID NO: 856 - protein, 10 aa, Homo sapiens
CQSVCCQPTC

SEQ ID NO: 857 - protein, 10 aa, Homo sapiens
CQTICRSTCC

SEQ ID NO: 858 - protein, 10 aa, Homo sapiens
CQTTCCRPSC

SEQ ID NO: 859 - protein, 10 aa, Homo sapiens
CQTTCCRTTC

SEQ ID NO: 860 - protein, 10 aa, Homo sapiens
CRATCCRPSC

SEQ ID NO: 861 - protein, 10 aa, Homo sapiens
CRGCGPSCCA

SEQ ID NO: 862 - protein, 10 aa, Homo sapiens
CRPACCETTC

SEQ ID NO: 863 - protein, 10 aa, Homo sapiens
CRPACCQNTC

SEQ ID NO: 864 - protein, 10 aa, Homo sapiens
CRPCCWATTC

SEQ ID NO: 865 - protein, 10 aa, Homo sapiens
CRPICRPACC

SEQ ID NO: 866 - protein, 10 aa, Homo sapiens
CRPLCCQTTC

SEQ ID NO: 867 - protein, 10 aa, Homo sapiens
CRPQCCQSVC

SEQ ID NO: 868 - protein, 10 aa, Homo sapiens
CRPQCCQTTC

SEQ ID NO: 869 - protein, 10 aa, Homo sapiens
CRPRCCISSC

SEQ ID NO: 870 - protein, 10 aa, Homo sapiens
CRPSCCESSC

SEQ ID NO: 871 - protein, 10 aa, Homo sapiens
CRPSCCETTC

SEQ ID NO: 872 - protein, 10 aa, Homo sapiens
CRPSCCISSC

SEQ ID NO: 873 - protein, 10 aa, Homo sapiens
CRPSCCKPQC

SEQ ID NO: 874 - protein, 10 aa, Homo sapiens
CRPSCCMSSC

SEQ ID NO: 875 - protein, 10 aa, Homo sapiens
CRPSCCQTTC

SEQ ID NO: 876 - protein, 10 aa, Homo sapiens
CRPSCCRPSC

SEQ ID NO: 877 - protein, 10 aa, Homo sapiens
CRPSCCVSRC

SEQ ID NO: 878 - protein, 10 aa, Homo sapiens
CRPSCCVSSC

SEQ ID NO: 879 - protein, 10 aa, Homo sapiens
CRPTCCETTC

SEQ ID NO: 880 - protein, 10 aa, Homo sapiens
CRPTCCQNTC

SEQ ID NO: 881 - protein, 10 aa, Homo sapiens
CRPTCCQTTC

SEQ ID NO: 882 - protein, 10 aa, Homo sapiens
CRPVCCDPCS

SEQ ID NO: 883 - protein, 10 aa, Homo sapiens
CRPVCCQTTC

SEQ ID NO: 884 - protein, 10 aa, Homo sapiens
CRPVCQPACC

SEQ ID NO: 885 - protein, 10 aa, Homo sapiens
CRPVCRPACC

SEQ ID NO: 886 - protein, 10 aa, Homo sapiens
CRPVCRPTCC

SEQ ID NO: 887 - protein, 10 aa, Homo sapiens
CRPVCRSTCC

SEQ ID NO: 888 - protein, 10 aa, Homo sapiens
CRPYCCESSC

SEQ ID NO: 889 - protein, 10 aa, Homo sapiens
CRRPVCCDPC

SEQ ID NO: 890 - protein, 10 aa, Homo sapiens
CRSQCCQSVC

SEQ ID NO: 891 - protein, 10 aa, Homo sapiens
CRTTCCHPSC

SEQ ID NO: 892 - protein, 10 aa, Homo sapiens
CRTTCCQPIC

SEQ ID NO: 893 - protein, 10 aa, Homo sapiens
CRTTCCQPTC

SEQ ID NO: 894 - protein, 10 aa, Homo sapiens
CRTTCCRPSC

SEQ ID NO: 895 - protein, 10 aa, Homo sapiens
CRTTCCRTTC

SEQ ID NO: 896 - protein, 10 aa, Homo sapiens
CSCSSCGSCA

SEQ ID NO: 897 - protein, 10 aa, Homo sapiens
CSCSSCGSCG

SEQ ID NO: 898 - protein, 10 aa, Homo sapiens
CSCTSCGSCG

SEQ ID NO: 899 - protein, 10 aa, Homo sapiens
CSPACQPTCC

SEQ ID NO: 900 - protein, 10 aa, Homo sapiens
CSPGCQPTCC

SEQ ID NO: 901 - protein, 10 aa, Homo sapiens
CSPSCCQTTC

SEQ ID NO: 902 - protein, 10 aa, Homo sapiens
CSQCSCYKPC

SEQ ID NO: 903 - protein, 10 aa, Homo sapiens
CSQSNCCKPC

SEQ ID NO: 904 - protein, 10 aa, Homo sapiens
CSQSSCCKPC

SEQ ID NO: 905 - protein, 10 aa, Homo sapiens
CSSGCGSCCQ

SEQ ID NO: 906 - protein, 10 aa, Homo sapiens
CSSGCGSSCC

SEQ ID NO: 907 - protein, 10 aa, Homo sapiens
CSSGCQPACC

SEQ ID NO: 908 - protein, 10 aa, Homo sapiens
CSSSCCQPSC

SEQ ID NO: 909 - protein, 10 aa, Homo sapiens
CSTPCCQPTC

SEQ ID NO: 910 - protein, 10 aa, Homo sapiens
CSTTCCQPIC

SEQ ID NO: 911 - protein, 10 aa, Homo sapiens
CTAVVCRPCC

SEQ ID NO: 912 - protein, 10 aa, Homo sapiens
CTDSCTPSCC

SEQ ID NO: 913 - protein, 10 aa, Homo sapiens
CTPSCCQPAC

SEQ ID NO: 914 - protein, 10 aa, Homo sapiens
CTRPICEPCC

SEQ ID NO: 915 - protein, 10 aa, Homo sapiens
CTSSCTPSCC

SEQ ID NO: 916 - protein, 10 aa, Homo sapiens
CVPACSCSSC

SEQ ID NO: 917 - protein, 10 aa, Homo sapiens
CVPACSCTSC

SEQ ID NO: 918 - protein, 10 aa, Homo sapiens
CVPVCCKPVC

SEQ ID NO: 919 - protein, 10 aa, Homo sapiens
CVPVCCVPTC

SEQ ID NO: 920 - protein, 10 aa, Homo sapiens
CVPVCCVPVC

SEQ ID NO: 921 - protein, 10 aa, Homo sapiens
CVSCVSSPCC

SEQ ID NO: 922 - protein, 10 aa, Homo sapiens
CVSRCCRPQC

SEQ ID NO: 923 - protein, 10 aa, Homo sapiens
CVSSCCKPQC

SEQ ID NO: 924 - protein, 10 aa, Homo sapiens
CVSSCCQHSC

SEQ ID NO: 925 - protein, 10 aa, Homo sapiens
CVSSCCQPFC

SEQ ID NO: 926 - protein, 10 aa, Homo sapiens
CVSSCCQPSC

SEQ ID NO: 927 - protein, 10 aa, Homo sapiens
CVSSCCRPQC

SEQ ID NO: 928 - protein, 10 aa, Homo sapiens
CVSTCCRPTC

SEQ ID NO: 929 - protein, 10 aa, Homo sapiens
CVTRCCSTPC

SEQ ID NO: 930 - protein, 10 aa, Homo sapiens
CVTSCCQPAC

SEQ ID NO: 931 - protein, 10 aa, Homo sapiens
CVTSCCQPSC

SEQ ID NO: 932 - protein, 10 aa, Homo sapiens
CVYSCCQPFC

SEQ ID NO: 933 - protein, 10 aa, Homo sapiens
CVYSCCQPSC

SEQ ID NO: 934 - protein, 10 aa, Homo sapiens
GCCGCSEGCG

SEQ ID NO: 935 - protein, 10 aa, Homo sapiens
GCCGCSGGCG

SEQ ID NO: 936 - protein, 10 aa, Homo sapiens
GCCGCSRGCG

SEQ ID NO: 937 - protein, 10 aa, Homo sapiens
GCCRPITCCP

SEQ ID NO: 938 - protein, 10 aa, Homo sapiens
GCGSSCCQCS

SEQ ID NO: 939 - protein, 10 aa, Homo sapiens
GCGVPVCCCS

SEQ ID NO: 940 - protein, 10 aa, Homo sapiens
LCCPCQTTCS

SEQ ID NO: 941 - protein, 10 aa, Homo sapiens
PCCCLRPVCG

SEQ ID NO: 942 - protein, 10 aa, Homo sapiens
PCCCRPVTCQ

SEQ ID NO: 943 - protein, 10 aa, Homo sapiens
PCCCVRPVCG

SEQ ID NO: 944 - protein, 10 aa, Homo sapiens
PCCSQASCCV

SEQ ID NO: 945 - protein, 10 aa, Homo sapiens
PCCSQSRCCV

SEQ ID NO: 946 - protein, 10 aa, Homo sapiens
PCCSQSSCCK

SEQ ID NO: 947 - protein, 10 aa, Homo sapiens
PCCSQSSCCV

SEQ ID NO: 948 - protein, 10 aa, Homo sapiens
PCCWATTCCQ

SEQ ID NO: 949 - protein, 10 aa, Homo sapiens
QCSCCKPYCS

SEQ ID NO: 950 - protein, 10 aa, Homo sapiens
RCYVPVCCCK

SEQ ID NO: 951 - protein, 10 aa, Homo sapiens
SCCAPVYCCK

SEQ ID NO: 952 - protein, 10 aa, Homo sapiens
SCCISSSCCP

SEQ ID NO: 953 - protein, 10 aa, Homo sapiens
SCCVSSCRCP

SEQ ID NO: 954 - protein, 10 aa, Homo sapiens
SCGCSQCSCY

SEQ ID NO: 955 - protein, 10 aa, Homo sapiens
SCGLENCCCP

SEQ ID NO: 956 - protein, 10 aa, Homo sapiens
VCCGASSCCQ

SEQ ID NO: 957 - protein, 10 aa, Homo sapiens
VCCGDSSCCQ

SEQ ID NO: 958 - protein, 11 aa, Homo sapiens
CASSCCTPSCC

SEQ ID NO: 959 - protein, 11 aa, Homo sapiens
CCCPSCVVSSC

SEQ ID NO: 960 - protein, 11 aa, Homo sapiens
CCCPSYCVSSC

SEQ ID NO: 961 - protein, 11 aa, Homo sapiens
CCCSSGCGSSC

SEQ ID NO: 962 - protein, 11 aa, Homo sapiens
CCDTCPPPCCK

SEQ ID NO: 963 - protein, 11 aa, Homo sapiens
CCEPHCCALSC

SEQ ID NO: 964 - protein, 11 aa, Homo sapiens
CCEPPCCAPSC

SEQ ID NO: 965 - protein, 11 aa, Homo sapiens
CCEPPCCATSC

SEQ ID NO: 966 - protein, 11 aa, Homo sapiens
CCETSCCQPSC

SEQ ID NO: 967 - protein, 11 aa, Homo sapiens
CCGSSCCGSGC

SEQ ID NO: 968 - protein, 11 aa, Homo sapiens
CCGSSCCGSSC

SEQ ID NO: 969 - protein, 11 aa, Homo sapiens
CCHPRCCISSC

SEQ ID NO: 970 - protein, 11 aa, Homo sapiens
CCHPSCCESSC

SEQ ID NO: 971 - protein, 11 aa, Homo sapiens
CCHPSCCISSC

SEQ ID NO: 972 - protein, 11 aa, Homo sapiens
CCHPSCCVSSC

SEQ ID NO: 973 - protein, 11 aa, Homo sapiens
CCHPTCCQNTC

SEQ ID NO: 974 - protein, 11 aa, Homo sapiens
CCHPTCCQTIC

SEQ ID NO: 975 - protein, 11 aa, Homo sapiens
CCISSCCKPSC

SEQ ID NO: 976 - protein, 11 aa, Homo sapiens
CCISSCCRPSC

SEQ ID NO: 977 - protein, 11 aa, Homo sapiens
CCISSSCCPSC

SEQ ID NO: 978 - protein, 11 aa, Homo sapiens
CCKAVCCVPTC

SEQ ID NO: 979 - protein, 11 aa, Homo sapiens
CCKPCCSQASC

SEQ ID NO: 980 - protein, 11 aa, Homo sapiens
CCKPCCSQSRC

SEQ ID NO: 981 - protein, 11 aa, Homo sapiens
CCKPCCSQSSC

SEQ ID NO: 982 - protein, 11 aa, Homo sapiens
CCKPCCSSSGC

SEQ ID NO: 983 - protein, 11 aa, Homo sapiens
CCKPCSCFSGC

SEQ ID NO: 984 - protein, 11 aa, Homo sapiens
CCKPCSCSSGC

SEQ ID NO: 985 - protein, 11 aa, Homo sapiens
CCKPCYCSSGC

SEQ ID NO: 986 - protein, 11 aa, Homo sapiens
CCKPICCVPVC

SEQ ID NO: 987 - protein, 11 aa, Homo sapiens
CCKPQCCQSVC

SEQ ID NO: 988 - protein, 11 aa, Homo sapiens
CCKPVCCKPIC

SEQ ID NO: 989 - protein, 11 aa, Homo sapiens
CCKPYCCQSSC

SEQ ID NO: 990 - protein, 11 aa, Homo sapiens
CCKPYCSQCSC

SEQ ID NO: 991 - protein, 11 aa, Homo sapiens
CCMPVCCKPVC

SEQ ID NO: 992 - protein, 11 aa, Homo sapiens
CCMPVCCKTVC

SEQ ID NO: 993 - protein, 11 aa, Homo sapiens
CCMSSCCKPQC

SEQ ID NO: 994 - protein, 11 aa, Homo sapiens
CCNPCCSQSSC

SEQ ID NO: 995 - protein, 11 aa, Homo sapiens
CCPGDCFTCCT

SEQ ID NO: 996 - protein, 11 aa, Homo sapiens
CCPSCVVSSCC

SEQ ID NO: 997 - protein, 11 aa, Homo sapiens
CCPSYCVSSCC

SEQ ID NO: 998 - protein, 11 aa, Homo sapiens
CCQNTCCRTTC

SEQ ID NO: 999 - protein, 11 aa, Homo sapiens
CCQPACCVSSC

SEQ ID NO: 1000 - protein, 11 aa, Homo sapiens
CCQPCCHPTCY

SEQ ID NO: 1001 - protein, 11 aa, Homo sapiens
CCQPCCRPTSC

SEQ ID NO: 1002 - protein, 11 aa, Homo sapiens
CCQPICGSSCC

SEQ ID NO: 1003 - protein, 11 aa, Homo sapiens
CCQPICVTSCC

SEQ ID NO: 1004 - protein, 11 aa, Homo sapiens
CCQPNCCRPSC

SEQ ID NO: 1005 - protein, 11 aa, Homo sapiens
CCQPSCCETSC

SEQ ID NO: 1006 - protein, 11 aa, Homo sapiens
CCQPSCCRPAC

SEQ ID NO: 1007 - protein, 11 aa, Homo sapiens
CCQPSCCSTPC

SEQ ID NO: 1008 - protein, 11 aa, Homo sapiens
CCQPSCCSTTC

SEQ ID NO: 1009 - protein, 11 aa, Homo sapiens
CCQPSCCVPSC

SEQ ID NO: 1010 - protein, 11 aa, Homo sapiens
CCQPSCCVSSC

SEQ ID NO: 1011 - protein, 11 aa, Homo sapiens
CCQPTCCHPSC

SEQ ID NO: 1012 - protein, 11 aa, Homo sapiens
CCQPTCCQPTC

SEQ ID NO: 1013 - protein, 11 aa, Homo sapiens
CCQPTCCRPRC

SEQ ID NO: 1014 - protein, 11 aa, Homo sapiens
CCQPTCCRPSC

SEQ ID NO: 1015 - protein, 11 aa, Homo sapiens
CCQPTCCRPTC

SEQ ID NO: 1016 - protein, 11 aa, Homo sapiens
CCQPTCCRTTC

SEQ ID NO: 1017 - protein, 11 aa, Homo sapiens
CCQPTCLSSCC

SEQ ID NO: 1018 - protein, 11 aa, Homo sapiens
CCQPTCLTSCC

SEQ ID NO: 1019 - protein, 11 aa, Homo sapiens
CCQPTCVASCC

SEQ ID NO: 1020 - protein, 11 aa, Homo sapiens
CCQPTCVTSCC

SEQ ID NO: 1021 - protein, 11 aa, Homo sapiens
CCQPYCHPTCC

SEQ ID NO: 1022 - protein, 11 aa, Homo sapiens
CCQSMCCQPTC

SEQ ID NO: 1023 - protein, 11 aa, Homo sapiens
CCQSNCCVPVC

SEQ ID NO: 1024 - protein, 11 aa, Homo sapiens
CCQSSCCKPCS

SEQ ID NO: 1025 - protein, 11 aa, Homo sapiens
CCQSSCCKPSC

SEQ ID NO: 1026 - protein, 11 aa, Homo sapiens
CCQSSCCKPYC

SEQ ID NO: 1027 - protein, 11 aa, Homo sapiens
CCQSSCCQSSC

SEQ ID NO: 1028 - protein, 11 aa, Homo sapiens
CCQSSCCVPVC

SEQ ID NO: 1029 - protein, 11 aa, Homo sapiens
CCQSSCFKPCC

SEQ ID NO: 1030 - protein, 11 aa, Homo sapiens
CCQSSCSKPCC

SEQ ID NO: 1031 - protein, 11 aa, Homo sapiens
CCQSSCYKPCC

SEQ ID NO: 1032 - protein, 11 aa, Homo sapiens
CCQSVCCQPTC

SEQ ID NO: 1033 - protein, 11 aa, Homo sapiens
CCQTICRSTCC

SEQ ID NO: 1034 - protein, 11 aa, Homo sapiens
CCQTTCCRPSC

SEQ ID NO: 1035 - protein, 11 aa, Homo sapiens
CCQTTCCRTTC

SEQ ID NO: 1036 - protein, 11 aa, Homo sapiens
CCRPACCETTC

SEQ ID NO: 1037 - protein, 11 aa, Homo sapiens
CCRPACCQNTC

SEQ ID NO: 1038 - protein, 11 aa, Homo sapiens
CCRPLCCQTTC

SEQ ID NO: 1039 - protein, 11 aa, Homo sapiens
CCRPQCCQSVC

SEQ ID NO: 1040 - protein, 11 aa, Homo sapiens
CCRPQCCQTTC

SEQ ID NO: 1041 - protein, 11 aa, Homo sapiens
CCRPSCCESSC

SEQ ID NO: 1042 - protein, 11 aa, Homo sapiens
CCRPSCCETTC

SEQ ID NO: 1043 - protein, 11 aa, Homo sapiens
CCRPSCCGSSC

SEQ ID NO: 1044 - protein, 11 aa, Homo sapiens
CCRPSCCISSC

SEQ ID NO: 1045 - protein, 11 aa, Homo sapiens
CCRPSCCKPQC

SEQ ID NO: 1046 - protein, 11 aa, Homo sapiens
CCRPSCCQTTC

SEQ ID NO: 1047 - protein, 11 aa, Homo sapiens
CCRPSCCVSRC

SEQ ID NO: 1048 - protein, 11 aa, Homo sapiens
CCRPSCCVSSC

SEQ ID NO: 1049 - protein, 11 aa, Homo sapiens
CCRPTCCQNTC

SEQ ID NO: 1050 - protein, 11 aa, Homo sapiens
CCRPTCCQTTC

SEQ ID NO: 1051 - protein, 11 aa, Homo sapiens
CCRPVCCDPCS

SEQ ID NO: 1052 - protein, 11 aa, Homo sapiens
CCRTTCCQPTC

SEQ ID NO: 1053 - protein, 11 aa, Homo sapiens
CCRTTCCRPSC

SEQ ID NO: 1054 - protein, 11 aa, Homo sapiens
CCRTTCCRTTC

SEQ ID NO: 1055 - protein, 11 aa, Homo sapiens
CCSCSSCGSCA

SEQ ID NO: 1056 - protein, 11 aa, Homo sapiens
CCSPGCQPTCC

SEQ ID NO: 1057 - protein, 11 aa, Homo sapiens
CCSQSSCCKPC

SEQ ID NO: 1058 - protein, 11 aa, Homo sapiens
CCSSGCGSCCQ

SEQ ID NO: 1059 - protein, 11 aa, Homo sapiens
CCSSGCGSSCC

SEQ ID NO: 1060 - protein, 11 aa, Homo sapiens
CCSTPCCQPTC

SEQ ID NO: 1061 - protein, 11 aa, Homo sapiens
CCVPACSCSSC

SEQ ID NO: 1062 - protein, 11 aa, Homo sapiens
CCVPACSCTSC

SEQ ID NO: 1063 - protein, 11 aa, Homo sapiens
CCVPICCKPIC

SEQ ID NO: 1064 - protein, 11 aa, Homo sapiens
CCVPICCKPVC

SEQ ID NO: 1065 - protein, 11 aa, Homo sapiens
CCVPVCCKPIC

SEQ ID NO: 1066 - protein, 11 aa, Homo sapiens
CCVPVCCKPVC

SEQ ID NO: 1067 - protein, 11 aa, Homo sapiens
CCVPVCCKSNC

SEQ ID NO: 1068 - protein, 11 aa, Homo sapiens
CCVPVCCKTVC

SEQ ID NO: 1069 - protein, 11 aa, Homo sapiens
CCVPVCCSSSC

SEQ ID NO: 1070 - protein, 11 aa, Homo sapiens
CCVPVCCVPVC

SEQ ID NO: 1071 - protein, 11 aa, Homo sapiens
CCVSSCCKPQC

SEQ ID NO: 1072 - protein, 11 aa, Homo sapiens
CCVSSCCQHSC

SEQ ID NO: 1073 - protein, 11 aa, Homo sapiens
CCVSSCCQPSC

SEQ ID NO: 1074 - protein, 11 aa, Homo sapiens
CCVSSCCRPQC

SEQ ID NO: 1075 - protein, 11 aa, Homo sapiens
CCVSTCCRPTC

SEQ ID NO: 1076 - protein, 11 aa, Homo sapiens
CCVSVCCKPVC

SEQ ID NO: 1077 - protein, 11 aa, Homo sapiens
CDSSCCQPSCC

SEQ ID NO: 1078 - protein, 11 aa, Homo sapiens
CEPCCRPVCCD

SEQ ID NO: 1079 - protein, 11 aa, Homo sapiens
CFKPCCCQSSC

SEQ ID NO: 1080 - protein, 11 aa, Homo sapiens
CGDGCCCPSCY

SEQ ID NO: 1081 - protein, 11 aa, Homo sapiens
CGGGCCGSSCC

SEQ ID NO: 1082 - protein, 11 aa, Homo sapiens
CGGSCCGSSCC

SEQ ID NO: 1083 - protein, 11 aa, Homo sapiens
CGLENCCCPSC

SEQ ID NO: 1084 - protein, 11 aa, Homo sapiens
CGQSCCRPACC

SEQ ID NO: 1085 - protein, 11 aa, Homo sapiens
CGQSCCRPVCC

SEQ ID NO: 1086 - protein, 11 aa, Homo sapiens
CGSCCQSSCCN

SEQ ID NO: 1087 - protein, 11 aa, Homo sapiens
CGSCGCSQCNC

SEQ ID NO: 1088 - protein, 11 aa, Homo sapiens
CGSCGCSQCSC

SEQ ID NO: 1089 - protein, 11 aa, Homo sapiens
CGSGCCGPVCC

SEQ ID NO: 1090 - protein, 11 aa, Homo sapiens
CGSGCCVPVCC

SEQ ID NO: 1091 - protein, 11 aa, Homo sapiens
CGSNCCQPCCR

SEQ ID NO: 1092 - protein, 11 aa, Homo sapiens
CGSSCCQPCCH

SEQ ID NO: 1093 - protein, 11 aa, Homo sapiens
CGSSCCQPCCR

SEQ ID NO: 1094 - protein, 11 aa, Homo sapiens
CGSSCCQPCYC

SEQ ID NO: 1095 - protein, 11 aa, Homo sapiens
CGSSCCQPSCC

SEQ ID NO: 1096 - protein, 11 aa, Homo sapiens
CGSSCCQSSCC

SEQ ID NO: 1097 - protein, 11 aa, Homo sapiens
CGSSCCVPICC

SEQ ID NO: 1098 - protein, 11 aa, Homo sapiens
CGSSCCVPVCC

SEQ ID NO: 1099 - protein, 11 aa, Homo sapiens
CGSSCSQCSCC

SEQ ID NO: 1100 - protein, 11 aa, Homo sapiens
CGVPVCCCSCS

SEQ ID NO: 1101 - protein, 11 aa, Homo sapiens
CHPRCCISSCC

SEQ ID NO: 1102 - protein, 11 aa, Homo sapiens
CHPSCCESSCC

SEQ ID NO: 1103 - protein, 11 aa, Homo sapiens
CHPSCCISSCC

SEQ ID NO: 1104 - protein, 11 aa, Homo sapiens
CHPTCCQNTCC

SEQ ID NO: 1105 - protein, 11 aa, Homo sapiens
CISSCCHPSCC

SEQ ID NO: 1106 - protein, 11 aa, Homo sapiens
CISSCCKPSCC

SEQ ID NO: 1107 - protein, 11 aa, Homo sapiens
CISSCCRPSCC

SEQ ID NO: 1108 - protein, 11 aa, Homo sapiens
CISSSCCPSCC

SEQ ID NO: 1109 - protein, 11 aa, Homo sapiens
CKPCCCSSGCG

SEQ ID NO: 1110 - protein, 11 aa, Homo sapiens
CKPCCSQASCC

SEQ ID NO: 1111 - protein, 11 aa, Homo sapiens
CKPCCSQSRCC

SEQ ID NO: 1112 - protein, 11 aa, Homo sapiens
CKPCCSQSSCC

SEQ ID NO: 1113 - protein, 11 aa, Homo sapiens
CKPQCCQSMCC

SEQ ID NO: 1114 - protein, 11 aa, Homo sapiens
CKPQCCQSVCC

SEQ ID NO: 1115 - protein, 11 aa, Homo sapiens
CKPVCCCVPAC

SEQ ID NO: 1116 - protein, 11 aa, Homo sapiens
CKPVCCKPICC

SEQ ID NO: 1117 - protein, 11 aa, Homo sapiens
CKPVCCMPVCC

SEQ ID NO: 1118 - protein, 11 aa, Homo sapiens
CKPVCCVPVCC

SEQ ID NO: 1119 - protein, 11 aa, Homo sapiens
CKPVCCVSVCC

SEQ ID NO: 1120 - protein, 11 aa, Homo sapiens
CKPYCSQCSCC

SEQ ID NO: 1121 - protein, 11 aa, Homo sapiens
CLPCCRPTCCQ

SEQ ID NO: 1122 - protein, 11 aa, Homo sapiens
CLTSCCQPSCC

SEQ ID NO: 1123 - protein, 11 aa, Homo sapiens
CMSSCCKPQCC

SEQ ID NO: 1124 - protein, 11 aa, Homo sapiens
CNPCCSQSSCC

SEQ ID NO: 1125 - protein, 11 aa, Homo sapiens
CPACCVSSCCQ

SEQ ID NO: 1126 - protein, 11 aa, Homo sapiens
CPESCCEPHCC

SEQ ID NO: 1127 - protein, 11 aa, Homo sapiens
CPESCCEPPCC

SEQ ID NO: 1128 - protein, 11 aa, Homo sapiens
CPSCCESSCCR

SEQ ID NO: 1129 - protein, 11 aa, Homo sapiens
CPSCCQTTCCR

SEQ ID NO: 1130 - protein, 11 aa, Homo sapiens
CPSCCVSSCCR

SEQ ID NO: 1131 - protein, 11 aa, Homo sapiens
CQCSCCKPYCS

SEQ ID NO: 1132 - protein, 11 aa, Homo sapiens
CQETCCRPSCC

SEQ ID NO: 1133 - protein, 11 aa, Homo sapiens
CQNTCCRTTCC

SEQ ID NO: 1134 - protein, 11 aa, Homo sapiens
CQPACCTASCC

SEQ ID NO: 1135 - protein, 11 aa, Homo sapiens
CQPACCTSSCC

SEQ ID NO: 1136 - protein, 11 aa, Homo sapiens
CQPACCTTSCC

SEQ ID NO: 1137 - protein, 11 aa, Homo sapiens
CQPACCVPVCC

SEQ ID NO: 1138 - protein, 11 aa, Homo sapiens
CQPACCVSSCC

SEQ ID NO: 1139 - protein, 11 aa, Homo sapiens
CQPCCHPTCCQ

SEQ ID NO: 1140 - protein, 11 aa, Homo sapiens
CQPCCRPACCE

SEQ ID NO: 1141 - protein, 11 aa, Homo sapiens
CQPCCRPACCQ

SEQ ID NO: 1142 - protein, 11 aa, Homo sapiens
CQPCCRPTCCQ

SEQ ID NO: 1143 - protein, 11 aa, Homo sapiens
CQPCYCPACCV

SEQ ID NO: 1144 - protein, 11 aa, Homo sapiens
CQPICCGSSCC

SEQ ID NO: 1145 - protein, 11 aa, Homo sapiens
CQPRCCETSCC

SEQ ID NO: 1146 - protein, 11 aa, Homo sapiens
CQPSCCETSCC

SEQ ID NO: 1147 - protein, 11 aa, Homo sapiens
CQPSCCRPACC

SEQ ID NO: 1148 - protein, 11 aa, Homo sapiens
CQPSCCVPSCC

SEQ ID NO: 1149 - protein, 11 aa, Homo sapiens
CQPSCCVSSCC

SEQ ID NO: 1150 - protein, 11 aa, Homo sapiens
CQPTCCCPSYC

SEQ ID NO: 1151 - protein, 11 aa, Homo sapiens
CQPTCCGSSCC

SEQ ID NO: 1152 - protein, 11 aa, Homo sapiens
CQPTCCHPSCC

SEQ ID NO: 1153 - protein, 11 aa, Homo sapiens
CQPTCCQPTCC

SEQ ID NO: 1154 - protein, 11 aa, Homo sapiens
CQPTCCRPSCC

SEQ ID NO: 1155 - protein, 11 aa, Homo sapiens
CQPTCCRPTCC

SEQ ID NO: 1156 - protein, 11 aa, Homo sapiens
CQPTCCRTTCC

SEQ ID NO: 1157 - protein, 11 aa, Homo sapiens
CQQACCMPVCC

SEQ ID NO: 1158 - protein, 11 aa, Homo sapiens
CQQACCVPICC

SEQ ID NO: 1159 - protein, 11 aa, Homo sapiens
CQQACCVPVCC

SEQ ID NO: 1160 - protein, 11 aa, Homo sapiens
CQQSCCVPVCC

SEQ ID NO: 1161 - protein, 11 aa, Homo sapiens
CQQSCCVSVCC

SEQ ID NO: 1162 - protein, 11 aa, Homo sapiens
CQSNCCVPVCC

SEQ ID NO: 1163 - protein, 11 aa, Homo sapiens
CQSSCCCPASC

SEQ ID NO: 1164 - protein, 11 aa, Homo sapiens
CQSSCCKPCCS

SEQ ID NO: 1165 - protein, 11 aa, Homo sapiens
CQSSCCKPCSC

SEQ ID NO: 1166 - protein, 11 aa, Homo sapiens
CQSSCCKPYCC

SEQ ID NO: 1167 - protein, 11 aa, Homo sapiens
CQSSCCNPCCS

SEQ ID NO: 1168 - protein, 11 aa, Homo sapiens
CQSSCCQSSCC

SEQ ID NO: 1169 - protein, 11 aa, Homo sapiens
CQSSCCVPVCC

SEQ ID NO: 1170 - protein, 11 aa, Homo sapiens
CQSSCFKPCCC

SEQ ID NO: 1171 - protein, 11 aa, Homo sapiens
CQSSCSKPCCC

SEQ ID NO: 1172 - protein, 11 aa, Homo sapiens
CQSSCYKPCCC

SEQ ID NO: 1173 - protein, 11 aa, Homo sapiens
CQSVCCQPTCC

SEQ ID NO: 1174 - protein, 11 aa, Homo sapiens
CQTTCCCPSCV

SEQ ID NO: 1175 - protein, 11 aa, Homo sapiens
CQTTCCRPSCC

SEQ ID NO: 1176 - protein, 11 aa, Homo sapiens
CQTTCCRTTCC

SEQ ID NO: 1177 - protein, 11 aa, Homo sapiens
CRPACCETTCC

SEQ ID NO: 1178 - protein, 11 aa, Homo sapiens
CRPACCQNTCC

SEQ ID NO: 1179 - protein, 11 aa, Homo sapiens
CRPCCCLRPVC

SEQ ID NO: 1180 - protein, 11 aa, Homo sapiens
CRPCCCVRPVC

SEQ ID NO: 1181 - protein, 11 aa, Homo sapiens
CRPCCWATTCC

SEQ ID NO: 1182 - protein, 11 aa, Homo sapiens
CRPLCCQTTCC

SEQ ID NO: 1183 - protein, 11 aa, Homo sapiens
CRPQCCQSVCC

SEQ ID NO: 1184 - protein, 11 aa, Homo sapiens
CRPQCCQTTCC

SEQ ID NO: 1185 - protein, 11 aa, Homo sapiens
CRPRCCISSCC

SEQ ID NO: 1186 - protein, 11 aa, Homo sapiens
CRPSCCESSCC

SEQ ID NO: 1187 - protein, 11 aa, Homo sapiens
CRPSCCISSCC

SEQ ID NO: 1188 - protein, 11 aa, Homo sapiens
CRPSCCKPQCC

SEQ ID NO: 1189 - protein, 11 aa, Homo sapiens
CRPSCCPSCCQ

SEQ ID NO: 1190 - protein, 11 aa, Homo sapiens
CRPSCCQTTCC

SEQ ID NO: 1191 - protein, 11 aa, Homo sapiens
CRPSCCRPQCC

SEQ ID NO: 1192 - protein, 11 aa, Homo sapiens
CRPSCCVSRCC

SEQ ID NO: 1193 - protein, 11 aa, Homo sapiens
CRPSCCVSSCC

SEQ ID NO: 1194 - protein, 11 aa, Homo sapiens
CRPTCCQNTCC

SEQ ID NO: 1195 - protein, 11 aa, Homo sapiens
CRPVCCCEPTC

SEQ ID NO: 1196 - protein, 11 aa, Homo sapiens
CRPVCCCYSCE

SEQ ID NO: 1197 - protein, 11 aa, Homo sapiens
CRTTCCHPSCC

SEQ ID NO: 1198 - protein, 11 aa, Homo sapiens
CRTTCCRPSCC

SEQ ID NO: 1199 - protein, 11 aa, Homo sapiens
CSCCKPYCSQC

SEQ ID NO: 1200 - protein, 11 aa, Homo sapiens
CSKPCCCQSSC

SEQ ID NO: 1201 - protein, 11 aa, Homo sapiens
CSPCCQPTCCR

SEQ ID NO: 1202 - protein, 11 aa, Homo sapiens
CSPCCVSSCCQ

SEQ ID NO: 1203 - protein, 11 aa, Homo sapiens
CSQCSCCKPCY

SEQ ID NO: 1204 - protein, 11 aa, Homo sapiens
CSQCSCYKPCC

SEQ ID NO: 1205 - protein, 11 aa, Homo sapiens
CSQSNCCKPCC

SEQ ID NO: 1206 - protein, 11 aa, Homo sapiens
CSQSSCCKPCC

SEQ ID NO: 1207 - protein, 11 aa, Homo sapiens
CSSSCCQPSCC

SEQ ID NO: 1208 - protein, 11 aa, Homo sapiens
CTPSCCQPACC

SEQ ID NO: 1209 - protein, 11 aa, Homo sapiens
CVASCCQPSCC

SEQ ID NO: 1210 - protein, 11 aa, Homo sapiens
CVPICCCKPVC

SEQ ID NO: 1211 - protein, 11 aa, Homo sapiens
CVPSCCQPCCH

SEQ ID NO: 1212 - protein, 11 aa, Homo sapiens
CVPVCCCKPMC

SEQ ID NO: 1213 - protein, 11 aa, Homo sapiens
CVPVCCCKPVC

SEQ ID NO: 1214 - protein, 11 aa, Homo sapiens
CVPVCCKPVCC

SEQ ID NO: 1215 - protein, 11 aa, Homo sapiens
CVSSCCKPQCC

SEQ ID NO: 1216 - protein, 11 aa, Homo sapiens
CVSSCCQHSCC

SEQ ID NO: 1217 - protein, 11 aa, Homo sapiens
CVSSCCQPCCH

SEQ ID NO: 1218 - protein, 11 aa, Homo sapiens
CVSSCCQPCCR

SEQ ID NO: 1219 - protein, 11 aa, Homo sapiens
CVSSCCQPFCC

SEQ ID NO: 1220 - protein, 11 aa, Homo sapiens
CVSSCCQPSCC

SEQ ID NO: 1221 - protein, 11 aa, Homo sapiens
CVSSCCRPQCC

SEQ ID NO: 1222 - protein, 11 aa, Homo sapiens
CVTRCCSTPCC

SEQ ID NO: 1223 - protein, 11 aa, Homo sapiens
CVTSCCQPACC

SEQ ID NO: 1224 - protein, 11 aa, Homo sapiens
CVTSCCQPSCC

SEQ ID NO: 1225 - protein, 11 aa, Homo sapiens
CVYSCCQPFCC

SEQ ID NO: 1226 - protein, 11 aa, Homo sapiens
CVYSCCQPSCC

SEQ ID NO: 1227 - protein, 11 aa, Homo sapiens
CYCPACCVSSC

SEQ ID NO: 1228 - protein, 11 aa, Homo sapiens
CYKPCCCQSSC

SEQ ID NO: 1229 - protein, 11 aa, Homo sapiens
CYKPCCCSSGC

SEQ ID NO: 1230 - protein, 11 aa, Homo sapiens
MCCCVPACSCS

SEQ ID NO: 1231 - protein, 11 aa, Homo sapiens
NCCVPVCCQCK

SEQ ID NO: 1232 - protein, 11 aa, Homo sapiens
QCSCCKPCYCS

SEQ ID NO: 1233 - protein, 11 aa, Homo sapiens
QCSCYKPCCCS

SEQ ID NO: 1234 - protein, 11 aa, Homo sapiens
SCCVPICCQCK

SEQ ID NO: 1235 - protein, 11 aa, Homo sapiens
SCCVPVCCQCK

SEQ ID NO: 1236 - protein, 11 aa, Homo sapiens
SCGCSQCNCCK

SEQ ID NO: 1237 - protein, 11 aa, Homo sapiens
SCGCSQCSCCK

SEQ ID NO: 1238 - protein, 11 aa, Homo sapiens
VCCCVPACSCS

SEQ ID NO: 1239 - protein, 11 aa, Homo sapiens
VCCCVPACSCT